(19) United States
(12) Patent Application Publication
(10) Pub. No.: US 2024/0082314 A1
(43) Pub. Date: Mar. 14, 2024

(54) WOUND HEALING AGENTS, METHODS FOR USE AND MANUFACTURE OF THE SAME

(71) Applicant: Eric Lewis, Tarzana, CA (US)

(72) Inventor: Eric Lewis, Tarzana, CA (US)

(21) Appl. No.: 18/465,083

(22) Filed: Sep. 11, 2023

Related U.S. Application Data
(60) Provisional application No. 63/405,373, filed on Sep. 9, 2022.

(51) Int. Cl. A61K 35/618; A61K 9/00; A61K 35/616; A61P 17/02

(52) U.S. Cl. CPC A61K 35/618; A61K 9/0014; A61K 35/616; A61P 17/02

(57) ABSTRACT

This invention provides new products, kits comprising the products, methods for treating conditions using the products, and methods for manufacturing the products. The products comprise an extract, a substrate, and/or a dressing. The extract may comprise a bivalve extract and/or an echinoderm extract.

### CROSS REFERENCE TO RELATED APPLICATIONS

This application claims priority to U.S. Provisional Application No. 63/405,373 filed on 9 Sep. 2022, the contents of which are hereby incorporated by reference in their entirety.

### TECHNICAL FIELD

The invention relates to the field of wounds, wound treatment, and cosmetics.

### BACKGROUND

Wound healing is a complex process that requires a highly regulated series of events including inflammation, tissue formation, revascularization and tissue remodeling. However, this is impaired in certain pathological conditions such as thrombosis, ischemia, diabetes and infection.

Refractory wounds, i.e., wounds where no improvement is observed for two to four weeks, can present insurmountable management challenges. Even when such wounds heal, the damaged skin often forms hypertrophic scars, marked by raised, itchy and inelastic skin, dark red or purple-colored tissue, and hypersensitivity. Areas of severe scars can develop into contractures and decrease joint mobility and result in chronic muscle and joint pain. Subjects with such conditions typically suffer psychological damage from feeling of self-consciousness which can reduce quality of life following an injury.

What is needed is a wound treatment that can facilitate healing of recalcitrant wounds and result in healthy looking skin.

### SUMMARY OF THE INVENTION

According to various aspects, the invention provides products, marine extract formulations, kits, methods of manufacturing such, and methods of using such to treat a subject.

A first aspect of the invention provides a product comprising a marine extract formulation. Optionally, the marine extract formulation comprises a bivalve extract and an echinoderm extract. Optionally, the product further comprises one or both of a substrate (e.g. collagen substrate) and a dressing.

A second aspect of the invention provides a product comprising a substrate (e.g. collagen substrate). Optionally, the product further comprises one or both of a dressing and an extract. According to some embodiments, the extract can comprise a dermatologically acceptable and therapeutically effective extract for treating a skin condition. According to some embodiments, the extract can comprise an extract of a marine animal. According to some embodiments, the extract can comprise one or both of a bivalve extract and an echinoderm extract. According to some embodiments, the extract can be a marine extract formulation.

According to some embodiments, in the first aspect or second aspect, the product can have any one or any of the following technical features.

The product can be provided as a kit in which components thereof (e.g. extract, substrate (e.g. collagen substrate), and/or dressing) are provided in respective containers or are otherwise separated or unmixed. In alternative embodiments, two or more components of the product are provided together in a container and, for example, mixed together or layered on one another.

The product can be configured for application to a wound or components of the product (e.g. extract, substrate (e.g. collagen substrate), and/or dressing) can be configured for application to a wound. The wound can be a wound cavity. In alternative embodiments, the wound can be a wound other than a wound cavity. In yet alterative embodiments, the product or components thereof can be configured for application to a site other than a wound.

The product can comprise a substrate (e.g. collagen substrate), an extract (e.g. a marine extract formulation), and a dressing, wherein the substrate (e.g. collagen substrate) and the extract can be applied to the wound (e.g. a wound cavity) and then covered with a dressing having an adhesive and a very high moisture vapor transmission such that the dressing can be kept in place for one week or more.

The product can comprise three layers: a substrate (e.g. collagen substrate) layer, an extract (e.g. MEF) layer, and a dressing layer. Optionally, the dressing and/or substrate (e.g. collagen substrate) layer comprises perforations. The substrate (e.g. collagen substrate) can be in the form of a sheet (e.g. pad), particles, or gel, or a combination thereof (e.g. a sheet and particles).

A third aspect of the invention provides a method of treating a condition comprising administering a product of the first aspect to a subject in need thereof.

A fourth aspect of the invention provides a method of treating a condition comprising administering a product of the second aspect to a subject in need thereof.

According to some embodiments, in the third aspect or fourth aspect, the method can have any one or any of the following technical features.

The condition can be a wound. The wound can be a wound cavity. In alternative embodiments, the wound can be a wound other than a wound cavity. In yet alterative embodiments, the condition can be a condition other than a wound.

The step of administering can comprise administering the product to a site having the condition.

The step of administering can comprise administering the substrate (e.g. collagen substrate) in or on the wound-side, with the extract (e.g. MEF) sandwiched between the substrate (e.g. collagen substrate) and dressing. Alternatively, the step of administering can comprise administering, in or on the wound, a mixture of the extract (e.g. MEF) and the substrate (e.g. collagen substrate) (e.g., substrate (e.g. collagen substrate) particles mixed with extract). Alternatively, the step of administering can comprise administering, in or on the wound, the dressing and applying one or both of the substrate (e.g. collagen substrate) and the extract (e.g. a mixture of the substrate (e.g. collagen substrate) and the extract) distal (outside) of the dressing. Alternatively, the invention contemplates embodiments having any of said administration steps except that one or both of the substrate (e.g. collagen substrate) and the dressing are omitted altogether (e.g., the product comprises MEF alone or MEF+either substrate (e.g. collagen substrate) or dressing). In any of such embodiments, the substrate may e provided in particle form or sheet form, or may be provided as a combination of both (e.g. in which a sheet is placed in a wound and the particles are filled about a gap(s) between the sheet and the periphery of the wound).

A fifth aspect of the invention provides a method of manufacturing a product of the first aspect. The method comprises obtaining an extract from a first organism and obtaining an extract from a second organism. The method can further comprise freezing and/or lyophilizing the extract of the first organism and/or the extract of the second organism. The step of obtaining an extract from the first organism can comprise obtaining a solvent extract from the first organism and/or disrupting tissue of the first organism. The step of obtaining an extract from the second organism can comprise obtaining a solvent extract from the second organism and/or disrupting tissue of the second organism. The first organism and the second organism can be a bivalve and an echinoderm, respectively. The method can further comprise preparing one or both of the first organism and the second organism for the extraction. The preparing can comprise freezing and/or lyophilizing one or both of the first organism and the second organism, or a tissue thereof.

A sixth aspect of the invention provides a method of manufacturing a product of the second aspect. The method comprises providing a substrate (e.g. collagen substrate). The method can further comprise providing a dressing. The method can further comprise providing an extract. The step of providing an extract can comprise obtaining at least one extract from at least one organism. The method can further comprise preparing the at least one organism for the extraction. The preparing can comprise freezing and/or lyophilizing the at least one organism, or a tissue thereof. The method can further comprise freezing and/or lyophilizing the extract.

Other aspects of the invention include aspects provided by omitting or substituting or a component (e.g. extract, substrate (e.g. collagen substrate), or dressing) of the product or method of any of the aforementioned aspects. For example, one aspect is provided by substituting the marine extract formulation of the first aspect with an extract of a bivalve or an extract of an echinoderm.

### BRIEF DESCRIPTION OF THE DRAWINGS

FIG. 1 is a photograph of an object wound

FIG. 2 is a photograph of the implantation of a collagen substrate (pellets) mixed with MEF.

FIG. 3 is a photograph of the implantation from FIG. 3 covered with gauze and a bandage,

FIG. 4 is a photograph showing wound healing.

FIG. 5 is a photograph showing wound healing.

FIG. 6 is a photograph showing wound healing.

FIG. 7 is a photograph of a wound.

FIG. 8 is a photograph of the implantation of a collagen substrate (pad) mixed with MEF.

FIG. 9 is a photograph of the implantation from FIG. 8 covered with a perforated dressing as set forth above,

FIG. 10 is a photograph of the wound from FIG. 9, 3 weeks later when the dressing had started to fall off.

FIG. 11 is a photograph showing wound healing.

FIG. 12 is a photograph showing (left panel) collagen substrate pellets and lyophilized MEF placed together; and (right panel) after substrate collagen pellets and MEF were mixed.

FIG. 13 depicts a product comprising a collagen substrate having a layer of extract (in green) provided thereon.

FIG. 14 depicts two products comprising a collagen substrate having a layer of extract (in green) provided thereon, one product having relatively small particles of MEF (left) and one product having relatively large particles of MEF (right).

FIG. 15 depicts six products comprising channeled collagen substrate layered with MEF (MEF layer on the underside and in the channels, slightly visible (in green) through the channels).

FIG. 16 depicts various techniques for creating channels of various sizes.

FIG. 17 depicts a close up of two products having a collagen substrate having channels. MEF is slightly visible in the channels.

FIG. 18 depicts two products having a collagen substrate having channel and layered with MEF and tools creating channels of various shapes (conical, elliptical, and frayed).

FIG. 19 depicts a kit made from a container having two products contained therein. Each product comprises a collagen substrate and a layer of MEF provided thereon.

### DETAILED DESCRIPTION OF THE INVENTION

Technical features described in this application can be used to construct various embodiments of the present invention, such as any of the first aspect through sixth aspect (or other aspect) taught above. In a first aspect of the present invention, a product comprising marine extract formulation is provided. In a second aspect of the present invention, a product comprising a substrate is provided. In a third aspect of the present invention, a method of treatment is provided comprising administering the product of the first aspect to treat a condition. In a fourth aspect of the present invention, a method of treatment is provided comprising administering the product of the second aspect to treat a condition. In a fifth aspect of the present invention, a method of manufacturing the product of the first aspect is provided. In a sixth aspect of the present invention, a method of manufacturing the product of the second aspect is provided. In a seventh aspect, a product comprising an extract of a bivalve or an extract of an echinoderm is provided, optionally further comprising one or both of a substrate and a dressing. In an eighth aspect of the present invention, a method of manufacturing the product of the seventh aspect is provided. In a ninth aspect of the present invention, a method of treatment is provided comprising administering the product of the seventh aspect to treat a condition.

This application provides configurations and features that may be used alone or in combination to provide a method or product of the present invention, according to some embodiments. Accordingly, a product or method of the present invention can have any one or any combination of the configurations and/or features taught herein. For example, for any aspect or embodiment taught herein (e.g. as set forth above or below) referencing an extract, dressing, and/or substrate, the extract, dressing, and/or substrate can comprise any technical feature or be configured with any configuration taught herein with respect to an extract, dressing, or substrate, respectively (except where such a technical feature or configuration conflicts with the express limitations of said aspect or embodiment).

References to configurations and/or features taught herein are not intended to limit entire scope of the present invention thereto. For example, when the present disclosure describes that a product or method can have a certain feature or configuration, the present disclosure also contemplates embodiments which are not limited to such configuration and/or feature.

It is further noted that, while certain feature(s) and/or configuration(s) may be described with respect to a product of the present invention, the present invention also contemplates a method of treatment which uses a product having said feature(s) and/or configuration(s) and also contemplates a method of manufacture which produces a product having said features and/or configurations. Similarly, while certain feature(s) and/or configuration(s) may be described with respect to a product used in a method, the present invention also contemplates a product having said feature(s) and/or configuration, e.g., which may be used independently of said method. Similarly, while certain step(s) may be described with respect to a method of treatment, the present invention also contemplates a product which is configured for such a step(s) or such a method (or configured to be able to be used in such a step(s) or method). Similarly, while certain step(s) may be described with respect to a method of manufacture, the present invention also contemplates a product which is a product by process, i.e. a product of the step(s) or method. Similarly, while a product, or component thereof, may be described as being configured for an action(s) or a step(s), the present invention also contemplates a method of treatment in which uses the product, or component thereof, to preform the action(s) or step(s).

### Abbreviations and Definitions

As used herein, the following definitions and abbreviations apply.

“Dressing” according to the present invention, is a bandage, optionally comprising an adhesive. Optionally, the dressing has a very high moisture vapor transmission such that the dressing can be kept in place for one week or more, preferably three weeks or more. Optionally, the dressing comprises channels.

“ECM protein” means a protein derived from an extracellular matrix (ECM). The ECM can be, for example, a three-dimensional network comprising extracellular macromolecules and minerals, such as collagen, enzymes, glycoproteins and hydroxyapatite that provide structural and biochemical support to surrounding cells. The ECM protein can be provided as purified from the ECM, associated with the ECM, or synthesized based on a known sequence (e.g., having at least 70%, 80% or 90% sequence identity) of a naturally occurring ECM protein.

“Exemplary” (or “e.g.,” or “by example”) means a non-limiting example.

“Identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). For example, sequence identity can be calculated using the default values for BLASTP and BLASTN indicated by Madden T. (Appendices. 2008 Jun. 23 [Updated 2021 Jun. 24]. In: BLAST® Command Line Applications User Manual [Internet]. Bethesda (MD): National Center for Biotechnology Information (US); 2008-. Available from: https://www.ncbi.nlm.nih.gov/books/NBK279684/, e.g., see blastp and blastn application options.). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

“Marine Extract Formulation” (or “MEF”) according to the present invention, means a formulation comprising at least two extracts of marine animals or plants or both as set forth herein. The MEF may comprise a combination of bivalve (e.g. scallop) extract or an echinoderm (e.g. sea cucumber) extract. In alternative embodiments, an MEF can be substituted by an extract which doesn't necessarily comprise two extracts, such as an extract of any marine animal (e.g. a bivalve extract or an echinoderm extract).

“Substrate” means a material configured to be placed in a wound that provides a medium or scaffolding for infiltrating cells of the proliferative wound healing phase. Optionally, the substrate comprises an ECM protein (e.g. collagen), for example, in a majority amount.

“Treatment” (or “treat”) refers to preventative or therapeutic action for a condition with clinically relevant symptoms. A treatment can be deemed successful if the healing is accelerated when compared to substantially the same wound without application of the instant invention. The use of “treatment” also contemplates conditions of a wound that make the subject susceptible to microbial infection and a treatment is successful if microbial infection is prevented in a subject at risk, or if the clinical course of a microbial infection is altered (e.g., slowed progression or accelerated regression). The use of “treatment” also contemplates wounds where, after healing is complete, leaves the skin in an altered condition (e.g., discolored or scarred) and treatment is deemed successful if the skin, after treatment, has a more normal appearance than skin without the treatment.

“Wound” refers broadly to injuries to tissue. The injured tissue can comprise any one or any combination of the skin, dermis, subdermal tissue, subcutaneous tissue, muscle, ligaments, nerves, blood vessels, bone, and other structures initiated in different ways, for example, surgery, (e.g., open post-cancer resection wounds, e.g., removal of melanoma and breast cancer, etc.), contained post-operative surgical wounds, pressure sores (e.g., from extended bed rest), wounds induced by trauma, puncture wounds, scrapes, cuts, and burns. The term is not limiting with respect to the cause, e.g., a physical cause such as bodily positioning (e.g., as in bed sores) or an impact as with trauma, surgical process a chemical process such as a burn or exposure to a caustic chemical substance, or a biological cause such as a disease process, an aging process, an obstetric process, or any other manner of biological process.

### Extract

A product of the present invention can, according to some embodiments, comprise an extract. The product can further comprise a substrate and/or a dressing. A method of treating a condition of the present invention can, according to some embodiments, comprise administering an extract (or a product comprising a extract). A method of manufacturing a product of the present invention, according to some embodiments, can comprise obtaining an extract.

The extract may be configured as a wound-healing extract, for example, comprising therapeutics (e.g. proteins) extracted from an organism and that promote wound healing.

Optional details of the extract will now be described.

The extract can comprise at least a portion of an organism which has been removed from its naturally occurring location in the organism. For example, the organism can comprise proteins and/or tissues which can be decompartmentalized or otherwise extracted from their naturally occurring locations in the organism. The extract can comprise one or more proteins and/or one or more tissues in isolated form (i.e. isolated from the remaining portions of the organism). Additionally or alternatively, the extract can comprise one or more proteins and/or one or more tissues in non-isolated form, for example, as part of a mixture with other portions of the organism (e.g. the product of homogenization of an organism or other tissue- or cell-disruption process that decompartmentalizes one or more proteins and/or one or more tissues but does not remove portions of the organism from the resulting extract).

The extract (e.g. MEF) can be provided in solid form, liquid form, or semi-sold (e.g. paste) form.

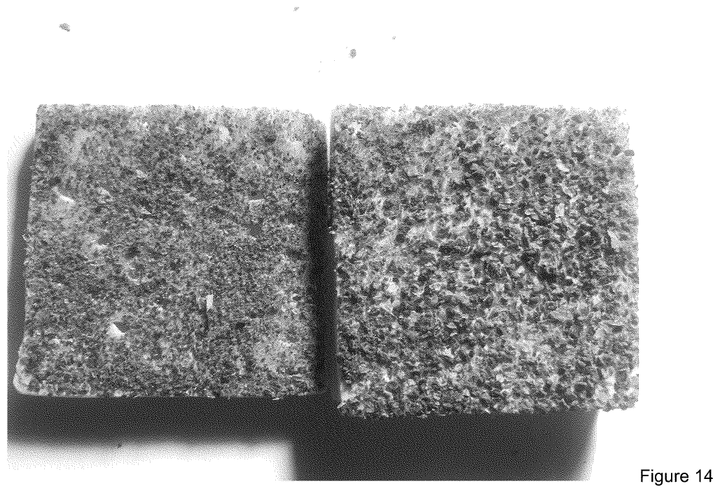

The extract (e.g. MEF) can be provided in dehydrated or dried (e.g. lyophilized) form, such as dried tissue. Such a form is sometimes referred to herein as dehydrated. The dried form can be free of water or can be have a water content of less than 20%, less than 10%, less than 5%, less than 2%, or substantially free of water. Products containing extract (e.g. dried ground echinoderm and/or bivalve) may be provided in dehydrated form, such as by lyophilization. The dried form of extract allows the selection of extract particle size to provide extended release by the selection of larger sized particles (see FIG. 14, right product, medium sized particle to provide moderate paced release (see FIG. 14, left product), or provided in powder form to provide immediate or rapid release. Dried extract can be configured to have a particle size with a diameter of the following: 0.05 mm to 2 mm, 0.05 mm to 1 mm, 0.05 mm to 0.5 mm, 0.1 mm to 2 mm, or 0.1 to 1 mm. Additionally or alternatively, dried extract can comprise extract in the form of powder having a particle size less than 0.05 mm. The diameter of a particle size can be evaluated by using a stack of sieves (each sieve possessing a different diameter opening) that separates feed particles according to diameter. For example, a 2 mm particle can pass through a 2 mm sieve but not a 1.5 mm sieve.

The inventor has discovered that the dried form of extract (e.g. MEF) is especially useful when combined with a substrate (e.g. a particulate substrate or sheet substrate) as taught herein as it can be placed in the wound with the substrate and remain there for multiple days or longer, such as 20 days or longer, such as 30 days. The dried form not only enhances the ability of the extract to remain in the wound but also provide sustained release of therapeutic wound-healing compounds over a prolonged period.

In addition, the inventor has discovered that the dried form of extract (e.g. MEF) is especially useful when combined with a substrate in sheet form having channels wherein the channels allow water exchange between the exterior of the sheet (i.e. distal side of the sheet) and wound (i.e. the proximal side of the substrate), i.e. the channels are configured (e.g. sized) to allow water to move from the surface of the wound through the channels to the wound bed where the extract has been placed under the substrate. The dried form of extract dissolves with time, but more specifically, the therapeutic compounds in the dried particles are solubilized and released while in the wound. After placing extract in the wound and covering with a channeled substrate, the user can periodically wash the wound, and the channels can be configured to allow water from the washing to enter the wound cavity and solubilize a portion of the dried extract. Thus, by combining a channeled substrate with an extract, washing or washing frequency can be used as a mechanism to release at least a portion of the therapeutic content from the extract.

In addition, the inventor has discovered that the dried form of extract (e.g. MEF) is especially useful when combined with a substrate in pellet form which allows water exchange the exterior of the sheet (i.e. distal side of the sheet) and the wound (i.e. the proximal side of the substrate), i.e. the pellets are configured (e.g. sized) to allow water to move from the surface of the wound through the pellets to the wound bed where the extract has been placed under the substrate or into the middle portion of the plurality of pellets if the extract has been mixed with the pellet. The dried form of extract dissolves with time, but more specifically, the dried particles are solubilized and released while in the wound. After placing extract in the wound and covering with a substrate in pellet form, the user can periodically wash the wound, and the small spaces between the pellets can be configured to allow water from the washing to enter the wound cavity (or central portion of the pellets if the extract has been placed within the pellet mixture) and solubilize a portion of the dried extract. Thus, by combining a pellet form substrate with an extract, washing or washing frequency can be used as a mechanism to release at least a portion of the therapeutic content from the extract.

In addition, the inventor has discovered that the dried form of extract (e.g. MEF) is especially useful when combined with a dressing having channels that can allow fluid exchange between the wound and the exterior. The dried form of extract dissolves with time, but more specifically, the dried particles are solubilized and released while in the wound. After placing extract in the wound and covering with a channeled dressing, the user can periodically wash the wound and the channels allow water from the washing to enter the wound cavity and solubilize a portion of the dried extract. Thus, by combining a channeled dressing with an extract, washing or washing frequency can be used as a mechanism to release at least a portion of the therapeutic content from the extract.

In addition, the dried form increases shelf life of the extract, concentrates the extract, enhances the ability of the extract to be combined with a substrate, allows the physician to avoid introducing additional moisture into the wound cavity, and prevents escape of extract from the wound due to inadvertent flowing out of the wound.

The extract (e.g. MEF) can comprise ground (e.g. homogenized) tissue.

The extract (e.g. MEF) can comprise a solvent extract of the organism. Additionally or alternatively, the extract can comprise a portion of the organism (e.g. an organ or tissue) removed from the organism. In alternative embodiments, the extract can be subtitled by comprise the whole organism in disrupted (e.g. ground, pulverized or homogenized) form.

The extract can comprise a bivalve extract and/or an echinoderm extract.

The extract can be therapeutically acceptable. The term therapeutically acceptable indicates that the extract is compatible, chemically and/or toxicologically, with the other ingredients in a formulation, and/or the mammal being treated therewith. For example, the extract can be therapeutically acceptable for administration to a mammal (e.g. human), such as therapeutically acceptable for administration to the skin or dermis (i.e. dermatologically acceptable).

The extract can be present in a formulation or composition in a therapeutically effective amount. The term therapeutically effective amount refers to an amount of the extract that is sufficient to affect treatment, when administered to a subject (e.g., a mammal, such as a human) in need of such treatment. The extract can be present in a therapeutically effective amount for administration to the epidermis, dermis, or subcutaneous layer of a mammal (e.g. human), for example, to treat any epidermal, dermal, or subcutaneous condition taught herein.

The extract can be present in a formulation or composition in a therapeutically effective concentration. The term therapeutically effective concentration refers to a concentration of the extract that is sufficient to affect treatment, when administered to a subject (e.g., a mammal, such as a human) in need of such treatment. The extract can be present in a therapeutically effective concentration for administration to the epidermis, dermis, or subcutaneous layer of a mammal (e.g. human), for example, to treat any condition taught herein.

The extract can be combined with a pharmaceutically acceptable carrier (e.g. diluent) to provide a formulation or composition. The term pharmaceutically acceptable carrier indicates that the carrier that is compatible with the other ingredients of the formulation and/or is not deleterious to the recipient thereof. For example, the pharmaceutically acceptable carrier can be a carrier that does not cause significant irritation to the skin of a mammal (e.g. human) and does not abrogate the biological activity and properties of the extract in treating a condition, such as any condition taught herein.

A product of the present invention can comprise a marine extract formulation as the extract. In alternative embodiments, the product can comprise any marine extract, i.e. not necessarily having an extract from two species. Such a product can exhibit remarkable therapeutic properties when combined with a substrate and/or dressing according to some embodiments. Accordingly, while the term “marine extract formulation” (or “MEF”) is used herein to illustrate the invention, the present invention alternatively contemplates products (or methods of their use or manufacture) in which the MEF is substituted by any extract, such as an extract of a marine animal (e.g. a bivalve extract or an echinoderm extract) or a marine plant (e.g. seaweed).

While certain marine extracts are discussed herein (e.g. bivalve and/or echinoderm), in some embodiments, the present invention is drawn to a product combining one or both of a substrate and a dressing with any dermatologically acceptable and therapeutically effective extract. For example, the extract can be any extract known for use in the treatment of a skin disorder. Examples of useful extracts are taught by Liu et al. (Front. Pharmacol., 22 Jul. 2022; Sec. Experimental Pharmacology and Drug Discovery; Volume 13-2022| https://doi.org/10.3389/fphar.2022.900439; The potential application of natural products in cutaneous wound healing: A review of preclinical evidence); EI-Sherbeni, S. A., Negm, W. A. (The wound healing effect of botanicals and pure natural substances used in in vivo models. Inflammopharmacol 31, 755-772 (2023). https://doi.org/10.1007/s10787-023-01157-5); and S. Ghuman et al. (Antioxidant, anti-inflammatory and wound healing properties of medicinal plant extracts used to treat wounds and dermatological disorders, South African Journal of Botany, Volume 126, 2019, Pages 232-240).

In a particular configuration, the extract can comprise a combination of a bivalve extract and an echinoderm extract. The bivalve extract can comprise a scallop (e.g. pink scallop) extract. The echinoderm extract can comprise a sea cucumber extract.

In another particular configuration, the extract can comprise a combination of a scallop (e.g. pink scallop) extract and one or both of a sea urchin (e.g. purple sea urchin) extract and an abalone extract (e.g. green abalone) or other gastropod extract.

In another particular configuration, the extract can comprise a combination of a cucumber extract and one or both of a sea urchin (e.g. purple sea urchin) extract and an abalone extract (e.g. green abalone) or other gastropod extract.

In another particular configuration, the extract comprises a sea urchin (e.g. purple sea urchin) extract and/or an abalone extract (e.g. green abalone) or other gastropod extract.

Additionally or alternatively to providing a sea cucumber extract, the extract can comprise a sea urchin (e.g. purple sea urchin) extract. Optionally, the extract further comprises abalone extract (e.g. green abalone) or other gastropod.

The extracts taught herein can be used by themselves or can be combined with any substrate (e.g. collagen substrate) taught herein, such as a substrate provided in sheet form or particle form.

A product of the present invention can comprise an extract formulated in any manner, e.g. as taught below. For example, the extract can be an MEF.

### Marine Extract Formulation

Among the embodiments taught herein is a marine extract formulation (MEF). An MEF may comprise one or both of a bivalve extract and an echinoderm extract. A marine extract formulation provides, according to some embodiments, a remarkably effective extract. Additionally or alternatively, the present invention contemplates a product comprising the marine extract formulation, a method of treating a condition by administering the marine extract formulation (or administering a product comprising the marine extract formulation), and a method of manufacturing the marine extract formulation (or manufacturing a product comprising the marine extract formulation).

Optional details of a marine extract formulation or other extract will now be described.

Inventor has discovered that the addition of a marine extract formulation to a wound can have a dramatically positively effect on the appearance of skin after the wound has healed.

Specifically, Inventor has discovered that combining an extract from an echinoderm and a bivalve (i.e., mollusk of the class Bivalvia) remarkably improves the appearance of the skin post healing.

MEFs according to the present invention can comprise an extract from two species or more. For example, an MEF can comprises an extract from an echinoderm and from two bivalves of differing orders, e.g., of the orders Pectinida and Venerida.

General methods for preparing extracts from marine organisms are taught in U.S. Pat. No. 10,772,825 issued Sep. 15, 2020 to Eric Lewis. Extracts and products of the present invention can be manufactured using such methods, by substituting the organism taught therein by an organism taught herein (e.g. bivalve or echinoderm) and carrying out the manufacturing methods except starting with an organism taught herein.

Additionally or alternatively, the present invention contemplates a product comprising an extract from any organism taught in U.S. Pat. No. 10,772,825.

### Formulation.

The MEF (or other extract, such as a bivalve extract or an echinoderm extract) can be formulated in any manner. For example, the MEF (or other extract) can be formulated for administration to a subject (e.g., human or mammal such as a wound thereof). For example, the MEF (or other extract) can be formulated as any of dried particles, cream, emulsion gel, plasma, lotion, ointment, powder, pad, capsule, liquid, spray, foam, solution, facial mask, skin mask. Any of such compositions can optionally be formulated for topical application.

Optionally, the bivalve extract and the echinoderm extract are provided as a homogenous mixture. Alternatively, the bivalve extract and the echinoderm extract can be provided as discrete components, for example, in layers.

Optionally, the formulation is provided as a solid, semi-solid, or liquid. Optionally, the formulation comprises the bivalve extract and the echinoderm extract and further comprises a diluent (e.g., a pharmaceutically acceptable diluent such as a polar or non-polar liquid), for example, formulated as an emulsion. Alternatively, the bivalve extract and the echinoderm extract are optionally lyophilized and the formulation is provided as a dry product (e.g., in sheet or powdered form).

Optionally, the formulation is combined with (e.g., layered onto or mixed with) a substrate taught herein (e.g., collagen substrate). Additionally or alternatively, the formulation can be layered onto a dressing taught herein. As one example, the formulation is layered onto a dressing and either mixed with the substrate or sandwiched (layered) between the substrate and the dressing. Additionally, the formulation can be provided within (e.g., injected into) a dressing or substrate.

As taught herein, an extract can comprise a plurality of extracts from respective organisms, such as a first organism (e.g. bivalve) and a second organism (e.g. echinoderm). The extract can comprise a mixture of the first organism extract and the second organism extract or can comprise each of the first organism extract and the second organism extract separated from each other (e.g. compartmentalized in separate containers).

### Lyophilization or Other Dehydration

Inventor has discovered that a useful form of MEF (or other extract such as a bivalve extract or an echinoderm extract) can be created by dehydrating the extract, such as by lyophilization. Optionally, the lyophilized MEF (or other extract) or other dehydrated MEF can be subjected to size reduction, e.g., wet milling, dry milling, jet milling, polytron, etc. It has been discovered that size reduction can result in better distribution of the MEF throughout the substrate.

The echinoderm extract and the bivalve can be mixed (or otherwise combined) and then dehydrated or each of the extracts can be dehydrated independently and then mixed (or otherwise combined).

The echinoderm extract and the bivalve can be mixed (or otherwise combined) and then lyophilized or each of the extracts can be lyophilized independently or mixed (or otherwise combined).

Accordingly, in some embodiments, the MEF is provided in dehydrated or lyophilized form.

### Bivalve Extract

A product of the present invention can, according to some embodiments, comprise a bivalve extract. The bivalve extract can be, or be part of, extract of any extract taught herein, e.g. a marine extract formulation. A method of treating a condition of the present invention can, according to some embodiments, comprise administering a bivalve extract (or a product comprising a bivalve extract). A method of manufacturing a product of the present invention, according to some embodiments, can comprise obtaining a bivalve extract.

Optional details of such a bivalve extract will now be described.

The bivalve extract can be obtained from any bivalve. Optionally, a bivalve is from an order selected from Actinodontida, Adapedonta, Afghanodesmatida, Arcida, Cardiida, Carditida, Colpomyida, Cyrtodontida, Fordillida, Hippuritida, Limida, Lucinida, Megalodontida, Modiomorphida, Myalinida, Myida, Mytilida, Nuculanida, Nuculida, Ostreida, Pectinida, Solemyida, Trigoniida, Tuarangiida, Unionida, and Venerida.

Optionally, the bivalve is a pectinida from a family selected from Anomiidae, Placunidae, Plicatulidae, Dimyidae, Entoliidae, Pectinidae, Propeamussiidae, and Spondylidae.

Optionally, the bivalve is a Adapedonta from a family selected from Edmondiidae, Hiatellidae, Pachydomidae, Pharidae, and Solenidae.

Optionally, the bivalve is a Venerida from an order selected from Anthracosiidae, Ferganoconchida, Shaanxiconchidae, Arcticidae, Pollicidae, Trapezidae, Veniellidae, Chamidae, Cyrenidae, Cyrenoididae, Glauconomidae, Glossidae, Kelliellidae, Lutetiidae, Vesicomyidae, Hemidonacidae, Anatinellidae, Cardiliidae, Mactridae, Mesodesmatidae, Galeommatidae, Kellidae, Lasaeidae, Leptonidae, Montacutidae, Palaeanodontidae, Prilukiellidae, Senderzoniellidae, Ungulinidae, Isocyprinidae, Neoleptonidae, and Veneridae

Optionally, the bivalve is a Pharidae from a genus selected from Afrophaxas, Cultellus, Ensiculus, Ensis, Leguminaria, Nasopharus, Neosiliqua, Novaculina, Orbicularia, Pharella, Pharus, Phaxas, Siliqua, Sinonovacula, Sinucultellus, and Sinupharus.

Optionally, the bivalve is a Pectinidae from a genus selected from Adamussium, Aequipecten, Amusium, Amussiopecten, Anguipecten, Annachlamys, Argopecten, Austrochlamys, Bractechlamys, Camptonectes, Caribachlamys, Chlamys, Ciclopecten, Complicachlamys, Coralichlamys, Crassadoma, Cryptopecten, Decatopecten, Delectopecten, Dentamussium, Equichlamys, Euvola, Excellichlamys, Flexopecten, Fortipecten, Glorichlamys, Gloripallium, Haumea (genus), Hemipecten, Hinnites, Hyalopecten, Juxtamusium, Laevichlamys, Leptopecten, Lissochlamys, Lissopecten, Lyropecten, Manupecten, Mesopeplum, Mimachlamys, Minnivola, Mirapecten, Miyagipecten, Mizuhopecten, Nodipecten, Notochlamys, Palliolum, Pascahinnites, Patinopecten, Pecten, Pedum, Placopecten, Pseudamussium, Pseudohinnites, Psychrochlamys, Scaeochlamys, Semipallium, Serratovola, Similipecten, Somalipecten, Spathochlamys, Swiftopecten, Talochlamys, Veprichlamys, Volachlamys, and Zygochlamys.

Optionally, the bivalve is a siliqua selected from Siliqua albida, Siliqua alta, Siliqua barnardi, Siliqua costata, Siliqua fasciata, Siliqua grayana, Siliqua herberti, Siliqua japonica, Siliqua lucida, Siliqua minima, Siliqua patula, Siliqua polita, Siliqua pulchella, Siliqua radiata, Siliqua rostrata, and Siliqua squama.

Optionally, the bivalve is of the genus Chlamys or genus Crassotrea. Examples of useful Chlamys bivalves include Chlamys rubida and Chlamys hastate. Examples of useful Crassotrea bivalves include Crassotrea corteziensis and Crassotrea columbiensis.

Optionally, the bivalve is selected from clams (e.g., siliqua patula), oysters (e.g. Crassostrea virginica), cockles (e.g. Cerastoderma edule), coquina (e.g. Donax variabilis or truncate donax), mussels (e.g. Mytilus edulis), and scallops (e.g. Chalmys rubida).

Optionally, the bivalve extract comprises an extract of any two of the aforementioned bivalves.

Optionally, an extract of any of the aforementioned bivalves is combined with an extract of an echinoderm.

Optionally, two bivalve extracts are selected from clams (e.g. siliqua patula) and scallops (e.g. Chalmys rubida) or oysters (e.g. Crassostrea virginica) and cockles (e.g. Cerastoderma edule), or coquina (e.g. Donax variabilis or truncate donax) and mussels (e.g. Mytilus edulis). Optionally these two bivalve extracts are further combined with an echinoderm extract. Optionally, these MEFs are combined with a collagen substrate.

The bivalve extract (e.g. scallop such as pink scallop) may be in the form of ground tissue (e.g. gonad tissue and/or a gland tissue). The ground tissue may be lyophilized tissue.

The bivalve extract (e.g. scallop such as pink scallop) can be obtained from the whole body of the bivalve or only a portion thereof. Examples of useful bivalve extracts include a visceral extract, gonad extract, digestive gland extract, testes extract, ovary extract or combination of such extracts.

The bivalve extract (e.g. scallop such as pink scallop) can comprise one or both of a gonad extract and a digestive gland extract. Such an extract can comprise disrupted gonad tissue and/or a digestive gland tissue, e.g. ground tissue. The gonad extract and/or a digestive gland tissue can comprise a majority (e.g. substantially all) of the bivalve extract present in the extract. The tissue can be lyophilized, e.g. before or after the grinding. The bivalve extract can be substantially free of another portion of the bivalve. The disrupted gonad tissue and/or a digestive gland tissue can comprise a majority (e.g. substantially all) of the bivalve tissue present in the extract.

The bivalve extract can be combined with any one or any combination of a sea cucumber extract, an abalone (e.g. green abalone) extract, and an urchin (e.g. purple urchin) extract.

The echinoderm extract can comprise a combination of sea cucumber extract and urchin (e.g. purple urchin extract). Such an extract can be combined with one or both of a scallop (e.g. pink scallop) extract and an abalone (e.g. green abalone) extract.

The bivalve extract can be a solvent extract. Alternatively, the bivalve extract can comprise disrupted bivalve tissue.

The bivalve (e.g. scallop such as pink scallop) can optionally be obtained by removing a portion of the bivalve (e.g. digestive gland and/or gonad)

The bivalve solvent extract can optionally be obtained by the steps of a) removing a tissue from the animal, b) breaking up the cells in the tissue, c) adding a solvent, d) separating the solvent fraction from a sediment fraction, and e) collecting the solvent fraction to obtain the bivalve extract. Optionally, the method comprises lyophilizing the bivalve tissue before performing the extraction process and/or lyophilizing the bivalve extract.

The bivalve extract can be a polar extract (e.g. obtained by extraction with a polar solvent) or a non-polar extract (e.g. obtained by extraction with a non-polar solvent). Examples of useful polar solvents include dimethyl sulfoxide (DMSO), isopropyl alcohol, ethanol, methanol, acetic acid and water. Examples of useful non-polar solvents are alkanes (e.g. hexane), chloroform and ether.

Optionally, a method of preparing bivalve tissue for exaction comprises a. The animal is removed from its shell. b. The visceral mass is separated from the adductor muscles. c. The visceral mass can be further prepared by removing the gonad. e. The bivalve is divided into 3 separate tissue samples—adductor muscle, viscera, and gonad.

In other embodiments, whole organism body or separated tissue samples as set forth above lyophilized. Optionally, the lyophilized tissues or whole organism are combined with other similarly processed organisms. Optionally, after said combination, the material is micronized or micronized by sonication, homgenization, milling, etc.

### Echinoderm Extract

A product of the present invention can, according to some embodiments, comprise an echinoderm extract. The product can further comprise one or both of a substrate and a dressing. The product can further comprise a bivalve extract. The echinoderm extract can be, or be part of, extract of any extract taught herein, e.g. a marine extract formulation. A method of treating a condition of the present invention can, according to some embodiments, comprise administering an echinoderm extract (or a product comprising a echinoderm extract). A method of manufacturing a product of the present invention, according to some embodiments, can comprise obtaining an echinoderm extract.

Optional details of such an echinoderm extract will now be described.

Optionally, the echinoderm is from a class other than Holothuroidea. Optionally, the echinoderm is from a class selected from Asteroidea (e.g. sea stars), Ophiuroidea (e.g. brittle stars), Echinoidea (e.g. sea urchins and sand dollars), and Crinoidea (e.g. sea lilies or feather stars).

Optionally, the echinoderm is from an order selected from Cidaroida, Cassiduloida, Spatangoida, Diadematoida, Echinothurioida, Pedinoida, Arbacioida, Echinoida, Phymosomatoida, Salenioida, Temnopleuroida, Clypeasteroida, and Holectypoida.

Optionally, the echinoderm is an Echinoida from a family selected from Echinidae, Echinometridae, Parasaleniidae, and Strongylocentrotidae.

Optionally, the echinoderm is a Strongylocentrotidae from a genus selected from Hemicentrotus, Mesocentrotus, Pseudocentrotus, and Strongylocentrotus.

Optionally, the echinoderm is selected from a starfish (e.g. Fromia monilis), a sea star, a feather star (e.g. Tropiometra), a brittle star (e.g. Ophiothrix fragilis) and an urchin (e.g. Stronylocentrotus purpuratus).

The echinoderm extract (e.g. sea cucumber) may be in the form of ground tissue (e.g. gonad tissue and/or a gland tissue). The ground tissue may be lyophilized tissue.

The echinoderm (e.g. sea cucumber) extract can be obtained from the whole body of the echinoderm or only a portion thereof.

The echinoderm extract (e.g. sea cucumber) can comprise one or both of a gonad extract and a digestive gland extract. Such an extract can comprise disrupted gonad tissue and/or a digestive gland tissue, e.g. ground tissue. The gonad extract and/or a digestive gland tissue can comprise a majority (e.g. substantially all) of the echinoderm extract present in the extract. The tissue can be lyophilized, e.g. before or after the grinding. The echinoderm extract can be substantially free of another portion of the echinoderm. The disrupted gonad tissue and/or a digestive gland tissue can comprise a majority (e.g. substantially all) of the echinoderm tissue present in the extract.

The echinoderm extract can be combined with one or both of a scallop (e.g. pink scallop) extract and an abalone (e.g. green abalone) extract.

The echinoderm extract can comprise a combination of sea cucumber extract and urchin (e.g. purple urchin extract). Such an extract can be combined with one or both of a scallop (e.g. pink scallop) extract and an abalone (e.g. green abalone) extract.

The echinoderm extract can be a solvent extract. Alternatively, the echinoderm extract can comprise disrupted echinoderm tissue.

The echinoderm solvent extract can optionally be obtained by the steps of a) providing tissue from the animal, b) breaking up the cells in the tissue, c) adding a solvent, d) separating the solvent fraction from a sediment fraction, and e) collecting the solvent fraction to obtain the bivalve extract. Optionally, the method comprises lyophilizing the echinoderm tissue before performing the extraction process and/or lyophilizing the echinoderm extract.

### Combinations

The MEF can comprise a combination of an echinoderm extract and a bivalve extract.

Optionally, the MEF comprises a combination of an echinoderm extract and a bivalve extract wherein the combination is selected from: the echinoderm is a starfish and the bivalve is a Actinodontida, the echinoderm is a starfish and the bivalve is an Adapedonta, the echinoderm is a starfish and the bivalve is an Afghanodesmatida, the echinoderm is a starfish and the bivalve is an Arcida, the echinoderm is a starfish and the bivalve is a Cardiida, the echinoderm is a starfish and the bivalve is a Carditida, the echinoderm is a starfish and the bivalve is a Colpomyida, the echinoderm is a starfish and the bivalve is a Cyrtodontida, the echinoderm is a starfish and the bivalve is a Fordillida, the echinoderm is a starfish and the bivalve is a Hippuritida, the echinoderm is a starfish and the bivalve is a Limida, the echinoderm is a starfish and the bivalve is a Lucinida, the echinoderm is a starfish and the bivalve is a Megalodontida, the echinoderm is a starfish and the bivalve is a Modiomorphida, the echinoderm is a starfish and the bivalve is a Myalinida, the echinoderm is a starfish and the bivalve is a Myida, the echinoderm is a starfish and the bivalve is a Mytilida, the echinoderm is a starfish and the bivalve is a Nuculanida, the echinoderm is a starfish and the bivalve is a Nuculida, the echinoderm is a starfish and the bivalve is an Ostreida, the echinoderm is a starfish and the bivalve is a Pectinida, the echinoderm is a starfish and the bivalve is a Solemyida, the echinoderm is a starfish and the bivalve is a Trigoniida, the echinoderm is a starfish and the bivalve is a Tuarangiida, the echinoderm is a starfish and the bivalve is an Unionida, the echinoderm is a starfish and the bivalve is a Venerida, the echinoderm is a sea star and the bivalve is an Actinodontida, the echinoderm is a sea star and the bivalve is an Adapedonta, the echinoderm is a sea star and the bivalve is an Afghanodesmatida, the echinoderm is a sea star and the bivalve is an Arcida, the echinoderm is a sea star and the bivalve is a Cardiida, the echinoderm is a sea star and the bivalve is a Carditida, the echinoderm is a sea star and the bivalve is a Colpomyida, the echinoderm is a sea star and the bivalve is a Cyrtodontida, the echinoderm is a sea star and the bivalve is a Fordillida, the echinoderm is a sea star and the bivalve is a Hippuritida, the echinoderm is a sea star and the bivalve is a Limida, the echinoderm is a sea star and the bivalve is a Lucinida, the echinoderm is a sea star and the bivalve is a Megalodontida, the echinoderm is a sea star and the bivalve is a Modiomorphida, the echinoderm is a sea star and the bivalve is a Myalinida, the echinoderm is a sea star and the bivalve is a Myida, the echinoderm is a sea star and the bivalve is a Mytilida, the echinoderm is a sea star and the bivalve is a Nuculanida, the echinoderm is a sea star and the bivalve is a Nuculida, the echinoderm is a sea star and the bivalve is an Ostreida, the echinoderm is a sea star and the bivalve is a Pectinida, the echinoderm is a sea star and the bivalve is a Solemyida, the echinoderm is a sea star and the bivalve is a Trigoniida, the echinoderm is a sea star and the bivalve is a Tuarangiida, the echinoderm is a sea star and the bivalve is an Unionida, the echinoderm is a sea star and the bivalve is a Venerida, the echinoderm is a feather star and the bivalve is an Actinodontida, the echinoderm is a feather star and the bivalve is an Adapedonta, the echinoderm is a feather star and the bivalve is an Afghanodesmatida, the echinoderm is a feather star and the bivalve is an Arcida, the echinoderm is a feather star and the bivalve is a Cardiida, the echinoderm is a feather star and the bivalve is a Carditida, the echinoderm is a feather star and the bivalve is a Colpomyida, the echinoderm is a feather star and the bivalve is a Cyrtodontida, the echinoderm is a feather star and the bivalve is a Fordillida, the echinoderm is a feather star and the bivalve is a Hippuritida, the echinoderm is a feather star and the bivalve is a Limida, the echinoderm is a feather star and the bivalve is a Lucinida, the echinoderm is a feather star and the bivalve is a Megalodontida, the echinoderm is a feather star and the bivalve is a Modiomorphida, the echinoderm is a feather star and the bivalve is a Myalinida, the echinoderm is a feather star and the bivalve is a Myida, the echinoderm is a feather star and the bivalve is a Mytilida, the echinoderm is a feather star and the bivalve is a Nuculanida, the echinoderm is a feather star and the bivalve is a Nuculida, the echinoderm is a feather star and the bivalve is an Ostreida, the echinoderm is a feather star and the bivalve is a Pectinida, the echinoderm is a feather star and the bivalve is a Solemyida, the echinoderm is a feather star and the bivalve is a Trigoniida, the echinoderm is a feather star and the bivalve is a Tuarangiida, the echinoderm is a feather star and the bivalve is a Unionida, the echinoderm is a feather star and the bivalve is a Venerida, the echinoderm is a brittle star and the bivalve is an Actinodontida, the echinoderm is a brittle star and the bivalve is an Adapedonta, the echinoderm is a brittle star and the bivalve is an Afghanodesmatida, the echinoderm is a brittle star and the bivalve is an Arcida, the echinoderm is a brittle star and the bivalve is a Cardiida, the echinoderm is a brittle star and the bivalve is a Carditida, the echinoderm is a brittle star and the bivalve is a Colpomyida, the echinoderm is a brittle star and the bivalve is a Cyrtodontida, the echinoderm is a brittle star and the bivalve is a Fordillida, the echinoderm is a brittle star and the bivalve is a Hippuritida, the echinoderm is a brittle star and the bivalve is a Limida, the echinoderm is a brittle star and the bivalve is a Lucinida, the echinoderm is a brittle star and the bivalve is a Megalodontida, the echinoderm is a brittle star and the bivalve is a Modiomorphida, the echinoderm is a brittle star and the bivalve is a Myalinida, the echinoderm is a brittle star and the bivalve is a Myida, the echinoderm is a brittle star and the bivalve is a Mytilida, the echinoderm is a brittle star and the bivalve is a Nuculanida, the echinoderm is a brittle star and the bivalve is a Nuculida, the echinoderm is a brittle star and the bivalve is an Ostreida, the echinoderm is a brittle star and the bivalve is a Pectinida, the echinoderm is a brittle star and the bivalve is a Solemyida, the echinoderm is a brittle star and the bivalve is a Trigoniida, the echinoderm is a brittle star and the bivalve is a Tuarangiida, the echinoderm is a brittle star and the bivalve is an Unionida, the echinoderm is a brittle star and the bivalve is a Venerida, the echinoderm is an urchin and the bivalve is an Actinodontida, the echinoderm is an urchin and the bivalve is an Adapedonta, the echinoderm is an urchin and the bivalve is an Afghanodesmatida, the echinoderm is an urchin and the bivalve is an Arcida, the echinoderm is an urchin and the bivalve is a Cardiida, the echinoderm is an urchin and the bivalve is a Carditida, the echinoderm is an urchin and the bivalve is a Colpomyida, the echinoderm is an urchin and the bivalve is a Cyrtodontida, the echinoderm is an urchin and the bivalve is a Fordillida, the echinoderm is an urchin and the bivalve is a Hippuritida, the echinoderm is an urchin and the bivalve is a Limida, the echinoderm is an urchin and the bivalve is a Lucinida, the echinoderm is an urchin and the bivalve is a Megalodontida, the echinoderm is an urchin and the bivalve is a Modiomorphida, the echinoderm is an urchin and the bivalve is a Myalinida, the echinoderm is an urchin and the bivalve is a Myida, the echinoderm is an urchin and the bivalve is a Mytilida, the echinoderm is an urchin and the bivalve is a Nuculanida, the echinoderm is an urchin and the bivalve is a Nuculida, the echinoderm is an urchin and the bivalve is an Ostreida, the echinoderm is an urchin and the bivalve is a Pectinida, the echinoderm is an urchin and the bivalve is a Solemyida, the echinoderm is an urchin and the bivalve is a Trigoniida, the echinoderm is an urchin and the bivalve is a Tuarangiida, the echinoderm is an urchin and the bivalve is a Unionida; or the echinoderm is an urchin and the bivalve is a Venerida.

Optionally, the combination of bivalve and echinoderm is selected from the following: the bivalve is a clam and the echinoderm is a sand dollar; the bivalve is an oyster and the echinoderm is a sand dollar; the bivalve is a cockle and the echinoderm is a sand dollar; the bivalve is a mussel and the echinoderm is a sand dollar; the bivalve is a scallop and the echinoderm is a sand dollar; the bivalve is an oyster and the echinoderm is a brittle star; the bivalve is a cockle and the echinoderm is a sea star; the bivalve is a cockle and the echinoderm is a feather star; the bivalve is a mussel and the echinoderm is a sea star; the bivalve is a scallop and the echinoderm is a starfish; the bivalve is a clam and the echinoderm is a starfish; the bivalve is a clam and the echinoderm is a brittle star; the bivalve is an oyster and the echinoderm is a sea star; the bivalve is a cockle and the echinoderm is a feather star; the bivalve is a coquina and the echinoderm is a feather star; the bivalve is a mussel and the echinoderm is an urchin; the bivalve is a cockle and the echinoderm is a brittle star; the bivalve is a cockle and the echinoderm is an urchin; the bivalve is a coquina and the echinoderm is a brittle star; the bivalve is a coquina and the echinoderm is an urchin; the bivalve is a mussel and the echinoderm is a brittle star; the bivalve is a scallop and the echinoderm is a brittle star; the bivalve is an oyster and the echinoderm is a starfish; the bivalve is an oyster and the echinoderm is a feather star; the bivalve is an oyster and the echinoderm is an urchin; the bivalve is a cockle and the echinoderm is an urchin; the bivalve is a cockle and the echinoderm is a brittle star; the bivalve is a coquina and the echinoderm is a brittle star; the bivalve is a scallop and the echinoderm is a sea star; the bivalve is a scallop (e.g. Chalmys rubida) and the echinoderm is an urchin (e.g. Stronylocentrotus purpuratus); the bivalve is a clam and the echinoderm is an urchin; the bivalve is a clam and the echinoderm is a feather star; the bivalve is a cockle and the echinoderm is a starfish; the bivalve is a cockle and the echinoderm is a sea star; the bivalve is a mussel and the echinoderm is a feather star; the bivalve is a clam and the echinoderm is a sea star; the bivalve is a cockle and the echinoderm is a starfish; the bivalve is a scallop and the echinoderm is a feather star; or the bivalve is a mussel and the echinoderm is a starfish.

### Extraction Process

The bivalve extract and/or echinoderm extract can be obtained by any extraction method.

For example, one example method follows: The tissue sample is obtained from the organism and stored on ice until a sufficient quantity of tissue is obtained. The tissue can either be frozen for processing at a future time or transferred directly to the next step in the process. Additionally or alternatively, the tissue can be processed drying (e.g. by freeze drying/lyophilization) to increase shelf life before extraction. The collected tissue is placed in a blender, homogenizer, grinder or similar machine to mechanically break up the cells in the tissue. Optionally, the tissue can be directly extracted with an appropriate polar and/or non-polar solvent (water, alcohol, hexane, chloroform, ether, or other suitable solvent) to obtain fractions containing active compounds or the tissue can be passed to the next step with extraction with a solvent. If the tissue was not dried after the collection step and processed as fresh tissue, the processed tissue is frozen or freeze dried or just dried. The dried tissue is ground to a desired size. The collagen can optionally be separated from the powder by screen filtering. The collagen gets caught in the screen, and the powder falls through. The collagen is optionally discarded. If the tissue sample has not already been extracted with solvents, the particulate may optionally extracted with polar and/or non-polar solvents to obtain fractions with the active compounds. The tissue sample can be centrifuged or filtered to remove cellular debris. Also, the solvent fraction can be further processed by column chromatography, HPLC or other similar separation technique. The powder is reconstituted in an appropriate carrier solvent or the fractions obtained by extracting the powder are mixed with a delivery vehicle. The reconstituted powder is used in the disclosed compositions.

In another example bivalve exact and the echinoderm exact is made by removing tissue (e.g. gland or organ such as the digestive gland and/or gonads) from the remaining portion of the animal, drying (e.g. by lyophilzation) the removed tissue and then grinding the dried tissue to size to provide a component of the extract. The bivalve exact and the echinoderm exact may each be processed independently before combining or the removed tissue from each animal can be processed together. In this example, ground tissue is used as the extract (i.e. the tissue is extracted from the animal). However, in alternative embodiments, although a whole animal homogenate may be used. In yet alternative embodiments, soluble components of the animal can be extracted using a solvent.

Optionally, one or both of the echinoderm and bivalve are extracted using a polar solvent.

Optionally, one or both of the echinoderm and bivalve are extracted using a non-polar solvent.

Optionally, one or both of the echinoderm and bivalve are extracted using a non-polar and a polar solvent (e.g., using different extraction steps for the polar and the non-polar solvents).

Optionally, one or both of the echinoderm and bivalve are extracted by tissue disruption. The tissue disruption can comprise homogenization or pulverization. Optionally, the tissue of the one or both of the echinoderm and bivalve is further extracted by solvent extraction, e.g., any solvent extraction steps described herein.

Optionally, one or both of the echinoderm and bivalve are prepared for extraction. Preparation of one or both of the echinoderm and bivalve can comprise removing a portion of the respective animal and then extracting a remaining portion of the respective animal. Preparation of the bivalve can comprise removing a shell from the bivalve before extracting a remaining portion of the bivalve. Preparation of one or both of the echinoderm and bivalve can comprise freezing and/or lyophilizing at least a portion of the echinoderm and/or bivalve before extracting at least a portion the echinoderm and/or bivalve which was subjected to freezing and/or lyophilizing.

Optionally, a method of preparing marine tissue for exaction comprises a. The body of the animal is sliced along the length of the body to expose the viscera. b. The gonad is separated visceral mass. c. The 5 muscles are removed from the body wall. e. The echinoderm is divided into 4 separate tissue samples—body wall, muscle, viscera, and gonad. Additionally or alternatively, the method of preparing can comprise the following process.

Optionally, the step of preparing can comprise a process in which a whole organism body or separated tissue sample(s) (e.g. as set forth above) are disrupted by freezing followed by lyophilized. Optionally, the lyophilized tissue or whole organisms are combined with other similarly processed organisms. Optionally, after said combination, the material is micronized or micronized by sonication, homogenization, milling, etc.

### Substrate

Some embodiments of the present invention comprise or use a substrate.

The substrate (e.g. collagen) can be a material that provides a medium or scaffolding for infiltrating granulation tissue. Non-limiting examples of substrates include natural extracellular matrix proteins (e.g., bovine, marine, or human) such as collagen (e.g., type I and/or III), fibronectin, vitronectin, other matrix proteins (or peptides thereof), or non-endogenous materials such as silk. The substrate may be, for example, combined with any extract taught herein to provide a product or kit, or used in a method of treatment.

The substrate can optionally comprise a polymeric structure which forms a network or matrix in vitro (before administration) or in vivo (after administration) and is optionally crosslinked to itself or another moiety. For example, collagen matrices are readily formed in-vitro by known techniques. Fibronectin, for example, forms a fibrillar matrix at the cell surface and is a substrate for Factor XIIIa (plasma transglutaminase) and can be cross-linked by Factor XIIIa to itself and the alpha-chain of fibrin. Vitronectin, for example, binds glycosaminoglycans, collagen, plasminogen and the urokinase-receptor.

The substrate can comprise a material that comprises one or more ECM proteins, collectively, in a substantial amount. For example, the substantial amount can be at least 20%, 30%, 40%, 50%, 60%, 70%, 80%, 90%, or 95% (w/w of the substrate). As another example, the substantial amount can be at least 20%, 30%, 40%, 50%, 60%, 70%, 80%, 90%, or 95% (w/w) of the dry weight of the substrate material.

In some embodiments, a product is provided comprising a substrate derived from an organism and the product further comprises an extract, wherein the substrate is derived from an organism other than an organism from which the substrate is derived.

In some embodiments, a product of the invention comprises an extract (e.g. MEF) and a substrate (e.g. collagen substrate). The combination of extract and substrate can be provided in as separated from each other or in contact with each other. The extract and substrate can be in contact with each other, for example, by layering the extract on the substrate (e.g. when the substrate is in sheet form), by impregnating the extract in the substrate (e.g. by injecting the substrate into a substrate, e.g. substrate in the form a sheet), or by mixing the substrate with the extract (e.g. when the substrate is provided in particulate form).

In some embodiments, the product comprises substrate (e.g. collagen substrate) impregnated or otherwise in contact with an extract (e.g. bivalve and/or echinoderm extract). Optionally, the extract is in dried (e.g. lyophilized) form. For example, the substrate can be in sheet form or particle form.

In some embodiments, the substrate (e.g. collagen substrate) can be configured in solid form. The substrate can be flexible or bendable (e.g. a flexible or bendable collagen substrate). The substrate can be elastic or viscoelastic (e.g. an elastic or viscoelastic collagen substrate).

In some embodiments, the substrate (e.g. collagen substrate) can be substantially non-flowable. For example, while the substrate can be in the form of a gel, the gel substrate can be configured such that it does not inadvertently flow out of a wound cavity (or does not flow through perforations optionally provided in a dressing or absorb through a dressing optionally provided over the substrate).

In some embodiments, the substrate (e.g. collagen substrate) has a viscosity of at least 100,000 centipoise (cps). For example, the substrate (e.g. collagen substrate) can have a viscosity of at least 200,000 cps, at least 300,000 cps, at least 400,000 cps, at least 500,000 cps, at least 1,000,000 cps, at least 10,000,000 cps, at least 20,000,000 cps, at least 30,000,000 cps, at least at least 40,000,000 cps, or at least at least 50,000,000 cps.

In some embodiments, the substrate (e.g. collagen substrate) is provided a a hydrogel or a dehydrated hydrogel. Optionally, the substrate (e.g. collagen substrate) is dehydrated and can be at least partially hydrated before inserting in a wound cavity. Optionally, the substrate (e.g. collagen substrate) has a state of hydration such that the substrate can absorb at least 5× or at least 10× (e.g. 5×-20×) its weight in water.

Optionally, the substrate is a type I collagen substrate in the form of a hydrogel and retains a native triple helix structure, e.g. upon dehydration and/or hydration.

### Sizing

The substrate, according to the present invention, can be a small size such as 8 mm 3 or smaller such that they can be sprinkled or poured into the wound (or wound cavity), for example, in powder or granular form. Accordingly, when provided in the form particles, a method of the invention can comprise inserting into a wound or otherwise applying to the wound a plurality of substrate particles. Such sized substrate particles are sometimes referred to herein as “pellets” for ease.

Alternatively, the substrate may be sized to greater than 8 mm 3 such that they can be positioned singularly in a wound cavity or placed adjacent to each other (or overlapping) to laterally fill the wound cavity. Alternatively, substrate can be provided in a larger size and cut to fit the wound cavity. Such sized substrates sometimes referred to herein as “pads” for ease.

Alternatively, the substrate may comprise a combination of 8 mm 3 or smaller substrate (e.g. collagen) and greater than 8 mm 3 substrate (e.g. collagen). For example, the substrate may comprise a sheet (e.g. collagen) and a plurality of pellets (e.g. collagen). Such a combination is especially useful for larger wounds (e.g. wounds wider than the lateral area of the sheet) and/or wounds with an irregular or non-uniform periphery (e.g. which may be difficult for the user to cut an identically shaped sheet to match the wound periphery). For such larger wounds, the user may perform a step of placing the substrate sheet into the wound and filling any gap(s) between the sheet and the periphery of the wound with the pellets.

Inventor has also discovered that embodiments comprising larger size substrates in combination with MEF and/or a dressing, can be useful and have a remarkable effect on healing recalcitrant wound and on wounds in general. In these larger size substrate products (e.g., 0.25-100 cm²), it has further been observed that therapeutic efficacy and skin appearance post-healing can be improved by creating macro-size pores (also called channels or perforations). Moreover, pores can be cylindrical or, to further draw in cells and fluid by surface tension or capillary action. As another example, pores can be elliptical. Pores can optionally be tapered (e.g., conical).

Substrate, according to the present invention, can be sized to fit within a prescribed area within the wound margin.

Optionally, the substrate (e.g., a collagen-based matrix) is provided for full thickness wounds as a sheet (or pad) with a thickness of 0.5 to 5 mm (e.g., 2-3 mm).

### Form

The substrate can be provided in any form. The substrate (e.g., collagen substrate) can optionally be provided as a liquid dispersion (e.g., aqueous dispersion, suspension, or colloid) or a solid (e.g., membrane or particulate) or semi-solid (e.g., gel). Useful forms of substrate (e.g., collagen-based substrate) include, for example, gels, pads, particles, pastes, powders, sheets, suspensions, emulsions, or solutions.

Optionally, the substrate is provided in dry form. In alternative embodiments, the substrate is provided in a partially hydrated form but is not a liquid or is not substantially flowable.

Optionally, the substrate is provided as a dried (e.g., lyophilized) or partially dried substrate which, when administered to a wound, hydrates from the wound to form a gel (e.g., from naturally occurring liquid discharged by the wound or surrounding tissue, or from a liquid applied exogenously).

Optionally, the substrate is provided as a gel. Optionally, the gel is provided from mixing a powder (e.g., collagen) or dried ECM protein (e.g., collagen) with water.

Optionally, the substrate is provided as a sponge. For example, a collagen-based sponge comprising insoluble forms of animal-derived collagen. Such a sponge can be configured to absorb large amounts of tissue exudate while adhering to a wound bed.

Optionally, the substrate (e.g., collagen-based matrix) is configured with a swelling ratio of between 2 and 10 (e.g., from 2.5 to 5) The swelling ratio is defined as the volume of water absorbed per unit volume of collagen-based matrix.

Optionally, the substrate comprises particles (e.g., collagen particles) having a particle size of about 200-2000 micrometers and optionally the substrate has a pore size is about 10-500 micrometers. Optionally, the substrate comprises particles (e.g., collagen particles) having a particle size of about 500-1800 micrometers or 800-1500 micrometers and optionally has a pore size of about 10-500 micrometers, or about 50-350 micrometers, respectively.

Optionally, the substrate is configured such that that after delivery, ability exists for the substrate to further expand and fill spaces within a wound bed as the substrate absorbs tissue fluids.

Optionally, the substrate is configured as a flowable substrate. For example, the flowable form may be configured to be shape-sustaining but readily deformable, e.g., those which behave like putty, to those which are gel like, to those which are runny. Optionally, the flowable substrate is configured is capable of passing through a syringe and cannula (e.g., of 1.6 mm diameter).

Optionally, the substrate is held in place by the dressing. Optionally, a product is provided comprising the substrate and the dressing, wherein the dressing is configured to hold the substrate in place for about 2 or more or 3 weeks or more. It has been discovered by Applicant that by protecting the integrity of the wound site (i.e. and not disrupting wound site and normal adjacent tissue by dressing changes), this configuration has unexpected outcomes in terms of efficacy and “time-to-resolution”.

Additionally or alternatively, a method of treating a condition comprises applying the substrate to the wound, applying the dressing over the substrate, and holding the substrate in place by the dressing for 2 or more or 3 or more weeks.

### Channels

Optionally, the substrate (e.g. collagen substrate) is provided in the form of a sheet and comprises channels.

The channels can optionally include cylindrical channels, conical channels, and/or elliptical channels. Useful channel configurations are taught, for example, in US 2021/0007899.

Optionally, the substrate comprises channel shapes that augment exchange of liquid water, water vapor, and/or air from the wound (under the substrate) to outside of the wound (over the substrate). Among the channel shapes that augment such exchange functions are those that are tapered (e.g., conical shape), elliptical shape (e.g., elliptical prism shaped), cylindrical shape, and star shape (e.g., star prism shaped).

Optionally, the channels include cylindrical channels and conical channels; cylindrical channels and elliptical channels, elliptical channels and conical channels; or cylindrical channels, elliptical channel and conical channels. The conical channels can be provided with the large end (noting cones are tapered) on the wound side or distal from the wound side (e.g., air side), or the substrate can be provided with both a plurality of channels with the large end on the wound side and a plurality of channels with the large end distal from the wound side (e.g., air side).

The instant channels can optionally be configured in a manner to optimize capillary action. It has been discovered in the mind of the inventor that such capillary action-optimized channels further facilitate wound repair.

For example, capillary action is increased when the channels are not perpendicular to the sheet, but at an angle, increasing the length of the channel through the sheet (e.g., the center or edge of the channel opening on the distal side is laterally offset from the center or edge of the corresponding channel opening on the proximal side).

Another capillary action-optimized channel is a channel in the shape of a cone, or other tapered channel. Dressings of the instant invention facilitate the movement of liquids from the distal side (e.g., topical agents) to the proximal side and liquids from the proximal side (e.g., exudates) to the distal side, cone-shaped channels of the instant invention are oriented in both directions (i.e., the larger diameter side of the cone oriented towards the distal side in some instance and towards the proximal side in other instances).

Moreover, Applicant has discovered that granulation tissue is drawn up into the substrate. The drawings up of granulation tissue can, according to some embodiments, be facilitated by the channels in the dressing of the present invention, thereby providing a protected environment for healing. By way of example, in one embodiment, the substrate comprises collagen with channels, the substrate is held in place with a dressing, according to the present invention. In such an embodiment, certain wound healing steps such as granulation proceeds in an accelerated manner. As stated elsewhere herein, the substrate seems to act like scaffolding for granulation tissue which is drawn in by forces greater than simple diffusion and can become incorporated into the healing wound by granulation tissue infiltrating the substrate. Without being bound by theory, Applicant believes that this process is responsible for the unexpectedly accelerated healing and qualitatively superior healing.

Another capillary action-optimized channel is elliptical in shape.

Optionally, channels of the instant invention comprise two or more of conical channels, elliptical channels, cylindrical channels, or star-shaped channels.

Optionally, capillary action-optimized channels of the instant invention have a diameter of less than 3 mm or less than 2 mm.

Optionally, the sheet (e.g. collagen sheet) comprises channels having a diameter of les than 0.5 (0.1-0.5 mm). When a substrate has channels of such diameter, the channels can optionally go all the way through the thickness of the sheet or can penetrate only part of the way through (e.g. 1-2 mm deep when the substrate is thicker than 1-2 mm). Without being bound by theory, the inventor belies that such fine channels provide a means for pulling granulosing tissue into a sheet, e.g., provided by enhanced capillary action.

Optionally, the sheet (e.g. collagen sheet) comprises channels having a diameter of 0.5-1 mm. Such channels can optionally go all the way through the thickness of the sheet. Without being bound by theory, the inventor belies that such medium sized holes provide an entrance for granulosing tissue and for fluid exchange between the wound and exterior of the sheet.

Optionally, the sheet (e.g. collagen sheet) comprises channels having a diameter of 1-2 mm. Such channels can optionally go all the way through the thickness of the sheet. The pacing of such channels can be, for example, about 4-6 mm measured center to center. Without being bound by theory, the inventor belies that such fine holes provide for enhanced fluid exchange between the wound and exterior of the sheet.

Optionally, the sheet comprises a first plurality of channels having a first diameter and a second plurality of channels having a second diameter, which is different than the first diameter. In one configuration, the first diameter may be 0.1-0.5 mm and the second diameter may be larger than 0.5 mm (e.g. 0.5-2 mm, 0.5-1 mm, or 1-2 mm). In another configuration, the first diameter may be 0.5-1 mm and the second diameter may be larger than the first diameter (e.g. 1-2 mm) or smaller than the first diameter (e.g. 0.1-0.5 mm). In another configuration, the first diameter may be 1-2 mm and the second diameter may be smaller than the first diameter (e.g. 0.1-1 mm or 0.5-1 mm). In any of such embodiments, the first diameter and second diameter can be substantially different diameters (e.g. the larger diameter may be at least 50% larger, or at least 100% larger than the smaller diameter).

Optionally, the sheet comprises a first plurality of channels having a first diameter, a second plurality of channels having a second diameter, and a third plurality of channels having a third diameter, wherein the first diameter is smaller than the second diameter, and the second diameter is smaller than the third diameter. In one configuration, the first diameter may be 0.1-0.5 mm. In another configuration, the second diameter may be 0.5-1 mm. In another configuration, the third diameter may be 1-2 mm. In another configuration, the first diameter may be 0.1-0.5 mm, the second diameter may be 0.5-1 mm, and the third diameter may be 1-2 mm.

### Extra Cellular Matrix Proteins

The substrate, according to the present invention, can comprise one or more natural extracellular matrix proteins (e.g., bovine, porcine, marine animal, equine, or human) such as collagen (e.g., type I and/or III), fibronectin and vitronectin.

Moreover, the instant substrate can comprise peptides of the forementioned extracellular matrix proteins. These peptides can be made by peptidase treatment (or other cleavage enzyme or method) or as harvested from the host animal.

Optionally, a useful ECM protein (e.g., collagen) is obtained from any source, e.g., a mammalian source such as bovine, human, porcine, equine, or ovine, or avian. The ECM protein can be obtained from any tissue of the source, e.g., tendon (e.g., flexor tendon).

Optionally, a useful ECM protein (e.g. collagen) is obtained from a marine animal.

### Collagen

Optionally, the substrate comprises one or more collagens. Any collagen can be used in the present invention. Useful collagens include, for example, one or more of type I-XX collagen. Optionally the substrate comprises soluble and/or insoluble collagen.

Optionally, the collagen comprises one or more soluble collagens of type I, II and III, e.g. prepared by limited enzymatic digestion of tissue enriched in such collagen types and are optionally formed into a collagen-based solution (i.e., a soluble collagen dissolved in a suitable solvent, such as dilute hydrochloric acid, dilute acetic acid or the like).

Optionally, the collagen comprises insoluble collagens derived from one or more of the following typical sources: type I collagen; bovine, chicken and fish skin, bovine and chicken tendons and bovine and chicken bones including fetal tissues; type II collagen: bovine articular cartilage, nasal septum, sternal cartilage; and type III collagen; bovine and human aorta and skin.

The collagen can natural, synthetic, semi-synthetic, or recombinant. Optionally, the collagen is modified collagens. Optionally, the collagen is present as a procollagen or a collagen-like protein or collagenous proteins comprising the motif (Gly-X-Y)n where n is an integer.

### Collagen Types

Examples of useful Type I collagens includes those, e.g. provided from as major fibrillar collagen of bone and skin. The Type I collagen can optionally be a heterotrimeric molecule comprising two α1 (I) chains and one α2(I) chain, encoded by the COL1A1 and COL1A2 genes, respectively.

Examples of useful Type II collagen include those provided as a homotrimeric collagen comprising three identical α1 (II) chains encoded by the COL2A1 gene. Purified type II collagen may be prepared from tissues by, methods known in the art, for example, by procedures described in Miller and Rhodes (1982) Methods In Enzymology 82: 33-64.

Examples of useful Type III collagens include those provided as a major fibrillar collagen (e.g., as found in skin and vascular tissues). Type III collagen is optionally provided as a homotrimeric collagen comprising three identical α1 (III) chains encoded by the COL3A1 gene. Methods for purifying type III collagen from tissues can be found in, for example, Byers et al. (1974) Biochemistry 13: 5243-5248; and Miller and Rhodes, supra.

Examples of useful Type IV collagens include those naturally found in basement membranes in the form of sheets rather than fibrils. Optionally the type IV collagen contains two α1 (IV) chains and one α2(IV) chain. Type IV collagen may be purified using, for example, the procedures described in Furuto and Miller (1987) Methods in Enzymology, 144: 41-61, Academic Press.

Examples of useful V collagen include a fibrillar collagen found in, primarily, bones, tendon, cornea, skin, and blood vessels. The Type V collagen can optionally be provided in homotrimeric and heterotrimeric forms. One form of type V collagen is a heterotrimer of two α1 (V) chains and one α2(V) chain. Another form of type V collagen is a heterotrimer of α1 (V), α2(V), and α3(V) chains. A further form of type V collagen is a homotrimer of α1 (V). Methods for isolating type V collagen from natural sources can be found, for example, in Elstow and Weiss (1983) Collagen Rel. Res. 3: 181-193, and Abedin et al. (1982) Biosci. Rep. 2: 493-502.

Examples of useful Type VI collagens include those having a small triple helical region and two large non-collagenous remainder portions. Type VI collagen is optionally a heterotrimer comprising α1 (VI), α2(VI), and α3(VI) chains. Type VI collagen is found in many connective tissues. Descriptions of how to purify type VI collagen from natural sources can be found, for example, in Wu et al. (1987) Biochem. J. 248: 373-381, and Kielty et al. (1991) J. Cell Sci. 99: 797-807.

Examples of useful Type VII collagens include a fibrillar collagen found in particular epithelial tissues. Type VII collagen is optionally a homotrimeric molecule of three α1 (VII) chains. Descriptions of how to purify type VII collagen from tissue can be found in, for example, Lunstrum et al. (1986) J. Biol. Chem. 261: 9042-9048, and Bentz et al. (1983) Proc. Natl. Acad. Sci. USA 80: 3168-3172. Type VIII collagen can be found in Descemet's membrane in the cornea. Type VIII collagen is a heterotrimer comprising two α1 (VIII) chains and one α2(VIII) chain, although other chain compositions have been reported. Methods for the purification of type VIII collagen from nature can be found, for example, in Benya and Padilla (1986) J. Biol. Chem. 261: 4160-4169, and Kapoor et al. (1986) Biochemistry 25: 3930-3937.

Examples of useful Type IX collagens include a fibril-associated collagen found in cartilage and vitreous humor. Type IX collagen is optionally a heterotrimeric molecule comprising α1 (IX), α2(IX), and α3 (IX) chains. Type IX collagen has been classified as a FACIT (Fibril Associated Collagens with Interrupted Triple Helices) collagen, possessing several triple helical domains separated by non-triple helical domains. Procedures for purifying type IX collagen can be found, for example, in Duance, et al. (1984) Biochem. J. 221: 885-889; Ayad et al. (1989) Biochem. J. 262: 753-761; and Grant et al. (1988) The Control of Tissue Damage, Glauert, A. M., ed., Elsevier Science Publishers, Amsterdam, pp. 3-28.

Examples of useful Type X collagens include a homotrimeric compound of α1 (X) chains. Type X collagen has been isolated from, for example, hypertrophic cartilage found in growth plates; see, e.g., Apte et al. (1992) Eur J Biochem 206 (1): 217-24.

Examples of useful Type XI collagens are those found in cartilaginous tissues associated with type II and type IX collagens, and in other locations in the body. Type XI collagen is optionally a heterotrimeric molecule comprising α1 (XI), α2(XI), and α3(XI) chains. Methods for purifying type XI collagen can be found, for example, in Grant et al., supra.

Examples of useful Type XII collagens include a FACIT collagen found primarily in association with type I collagen. Type XII collagen is optionally a homotrimeric molecule comprising three α1 (XII) chains. Methods for purifying type XII collagen and variants thereof can be found, for example, in Dublet et al. (1989) J. Biol. Chem. 264: 13150-13156; Lunstrum et al. (1992) J. Biol. Chem. 267: 20087-20092; and Watt et al. (1992) J. Biol. Chem. 267: 20093-20099.

Examples of useful Type XIII collagens include a non-fibrillar collagen found, for example, in skin, intestine, bone, cartilage, and striated muscle. A detailed description of type XIII collagen may be found, for example, in Juvonen et al. (1992) J. Biol. Chem. 267: 24700-24707.

Examples of useful Type XIV collagens include a homotrimeric molecule comprising α1 (XIV) chains. Methods for isolating type XIV collagen can be found, for example, in Aubert-Foucher et al. (1992) J. Biol. Chem. 267: 15759-15764, and Watt et al., supra.

Examples of useful Type XV collagens include a homologous in structure to type XVIII collagen. Information about the structure and isolation of natural type XV collagen can be found, for example, in Myers et al. (1992) Proc. Natl. Acad. Sci. USA 89: 10144-10148; Huebner et al. (1992) Genomics 14: 220-224; Kivirikko et al. (1994) J. Biol. Chem. 269: 4773-4779; and Muragaki, J. (1994) Biol. Chem. 264: 4042-4046.

Examples of useful collagens include a fibril-associated collagen, found, for example, in skin, lung fibroblast, and keratinocytes. Information on the structure of type XVI collagen and the gene encoding type XVI collagen can be found, for example, in Pan et al. (1992) Proc. Natl. Acad. Sci. USA 89: 6565-6569; and Yamaguchi et al. (1992) J. Biochem. 112: 856-863.

Examples of useful Type XVII collagens include a a hem idesmosal transmembrane collagen, also known at the bullous pemphigoid antigen. Information on the structure of type XVII collagen and the gene encoding type XVII collagen can be found, for example, in Li et al. (1993) J. Biol. Chem. 268(12): 8825-8834; and McGrath et al. (1995) Nat. Genet. 11(1): 83-86.

Examples of useful Type XVIII collagen include a protein similar structure to type XV collagen which can be isolated from the liver. Descriptions of the structures and isolation of type XVIII collagen from natural sources can be found, for example, in Rehn and Pihlajaniemi (1994) Proc. Natl. Acad. Sci USA 91: 4234-4238; Oh et al. (1994) Proc. Natl. Acad. Sci USA 91: 4229-4233; Rehn et al. (1994) J. Biol. Chem. 269: 13924-13935; and Oh et al. (1994) Genomics 19: 494-499.

Examples of useful Type XIX collagen include that which are described, for example, in Inoguchi et al. (1995) J. Biochem. 117: 137-146; Yoshioka et al. (1992) Genomics 13: 884-886; and Myers et al., J. Biol. Chem. 289: 18549-18557 (1994).

Examples of useful Type XX collagen include that which is found in chick cornea. (See, e.g., Gordon et al. (1999) FASEB Journal 13:A1119; and Gordon et al. (1998), IOVS 39:S1128.)

Optionally, the collagen is piscean collagen, e.g. as a protein extracted from fish waste, including the scales, skin or air bladders (e.g. from fresh water carp).

Optionally, the substrate comprises a mixture of Type I and Type III collagen. The ratio of Type III to Type I collagen is optionally equal to or greater than 30:70, 40:60, 45:55, 50:50, 55:45, 60:40, 65:35, 70:30, 75:25, 80:20, 85:15, 90:10, or 95:5. The ratio of Type III to Type I collagen is optionally equal to or less than 30:70, 40:60, 45:55, 50:50, 55:45, 60:40, 65:35, 70:30, 75:25, 80:20, 85:15, 90:10, or 95:5. Type III collagen is found in several types of connective tissue throughout the human body, with its proportion high in young tissue, such as tissue formed during the early phases of wound healing and can be provided as a majority or minority source of collagen in the mixture. Increasing the ratio of type I to type III collagen may be useful to enhance the durability and strength of the wound dressing.

Optionally, the substrate comprises collagen and Type III collagen is at least 30%, 40%, 50%, 60%, 70%, 80%, 90% or 95% of the weight or volume of the collagen component.

Optionally, the collagen is at least 30%, 40%, 50%, 60%, 70%, 80%, 90% or 95% of the weight or volume of the substrate. For example, the collagen may be any of the aforementioned collagens, e.g., Type I or Type III.

Optionally, the collagen is at least 30%, 40%, 50%, 60%, 70%, 80%, 90% or 95% of the dry weight or dry volume of the substrate (i.e. the weight or volume after dehydrading the substrate). For example, the collagen may be any of the aforementioned collagens, e.g., Type I or Type III.

Optionally, the substrate comprises collagen as a majority of the weight of the substrate, a majority of the volume of the substrate, a majority of the dry weight of the substrate, or a majority of the dry volume of the substrate. For example, the collagen may be any of the aforementioned collagens, e.g., Type I or Type III.

### Marine-Sourced Collagen

Optionally, the collagen substrate is derived from a marine animal. Marine animals that are useful sources of collagen for use as taught here include, for example, fish, such as tilapia. By example, collagen can be derived from fish bones, skin and/or scales. Marine (e.g. fish) collagen can be obtained from a marine animal and digested (e.g. by proteolytic enzyme such as a protease) into peptides to provide a substrate, e.g., which can be placed into wounds. Useful digestion enzymes include a metalloproteinase (e.g. matrix metalloproteinases (MMP) such as mammalian MMP), cysteine protease (e.g. mammalian), or a bacterial protease.

It has been discovered by the Applicant that marine-sourced collagen and collagen fragments as described herein, can be especially useful when used in combination with extracts taught herein.

### Collagen Characteristics

The collagen (e.g. Type I, II or III collagen) of the substrate can optionally be solubilized collagen or soluble collagen fragments, for example, having a molecular weight in the range from about 5,000 to about 100,000, or from about 5,000 to about 50,000. Additionally or alternatively, the collagen may also comprise solubilized collagen or soluble collagen fragments, for example, having a molecular weight in the range from about 5,000, about 6,000, about 7,000, about 8,000, about 9,000, about 10,000, about 12,000, about 14,000, about 16,000, about 18,000, about 20,000, about 22,000, about 24,000, about 26,000, about 28,000, about 30,000, about 32,000, about 34,000, about 36,000, about 38,000, about 40,000, about 42,000, about 44,000, about 46,000, about 48,000, about 50,000, about 52,000, about 54,000, about 56,000, about 58,000, about 60,000, about 62,000, about 64,000, about 66,000, about 68,000, about 70,000, about 72,000, about 74,000, about 76,000, about 78,000, about 80,000, about 82,000, about 84,000, about 86,000, about 88,000, about 90,000, about 92,000, about 94,000, about 96,000, about 98,000, about 100,000, or any range including and/or in between any two of the preceding values. Additionally or alternatively, the solubilized collagen or soluble collagen fragments may be obtained by pepsin treatment of a natural collagen. Additionally or alternatively, in the collagen may be obtained from bovine corium that has been rendered largely free of non-collagenous components, for example, including fat, non-collagenous proteins, polysaccharides, and other carbohydrates, as described in U.S. Pat. Nos. 4,614,794, 4,320,201, 6,309,454, 8,461,410, and EP Patent 1758638, each incorporated by reference herein in their entirety.

Optionally, the collagen is reduced antigenic collagen. Some forms of collagen (e.g. certain non-human sources such as bovine) can be antigenic for some recipients. Such antigenicity can optionally be reduced before use, e.g. by cleavage with an enzyme. For example, after digestion (e.g. pepsin), the cleaved peptide ends of the collagen can be discarded and only the central collagen bundles (tropocollagen) retained for use in the present invention. These central collagen bundles have greatly reduced antigenicity and they can be used for the purposes noted above without undue antigenic side effects.

Optionally, the collagen is treated by terminal sterilization, e.g. as disclosed U.S. patent application Ser. No. 11/383,845. Terminal sterilization can optionally be configured to reduce the level of active biological contaminants without adversely affecting the collagen material.

Optionally, the collagen is native collagen (e.g. type I) which retains a native triple helix structure. The triple helix may enhance binding with wound exudate growth factors and cytokines and may also ensure mechanical competence in the hydrated state e.g. hydration in vitro before administration or in vivo from exudate). Alternatively, the collagen may optionally be denatured collagen.

Optionally, the collagen substrate is dehydrated and can be at least partially hydrated before inserting in a wound cavity. Optionally, the collagen substrate has a state of hydration such that the substrate can absorb at least 5× or at least 10× (e.g. 5×-20×) its weight in water. Optionally, the collagen retains a native triple helix structure.

Optionally, the collagen substrate exhibits elastic or viscoelastic properties.

Optionally, the collagen substrate is water-insoluble.

Optionally, the collagen substrate is substantially non-fibrous.

Optionally, the substrate comprises hydrolyzed collagen, e.g. a collagen hydrolysate polypeptide having a molecular weight lower than native collagen, e.g. in the 100 to 300,000 Daltons range, and is derived by hydrolysis. Hydrolyzed collagen is commercially available in powdered form or an aqueous solution. Commercial preparation is typically accomplished by one of four methods: (1) alkaline hydrolysis; (2) enzymatic hydrolysis; (3) acid hydrolysis; and (4) synthetically by fermentation. Any of these methods can be used to derive the hydrolyzed collagen from either a bovine (bone and skin preferred), porcine, fish, avian or a synthetic source. Optionally the hydrolyzed collagen comprises Hydroxylysine and/or hydroxyproline amino acids in concentrations from 0.7 to 1.2 wt. % of hydrolyzed collagen.

Optionally, the collagen comprises Collagen fibrils, e.g. nanofibers composed of tropocollagen (triple helices of collagen molecules). Tropocollagens also include tropocollagen-like structures exhibiting triple helical structures. The collagen fibrils may have diameters ranging from 1 nm and 1 μm. For example, the collagen fibrils of the invention may have an average or individual fibril diameter ranging from 1, 2, 3, 4, 5, 10, 15, 20, 30, 40, 50, 60, 70, 80, 90, 100, 200, 300, 400, 500, 600, 700, 800, 900, or 1,000 nm (1 μm).

### Substrate Examples

Optionally, the substrate comprises a hydrolyzed collagen gel, e.g., made from a type I (e.g. bovine) collagen (e.g. sold under the tradename HYCOL®).

Optionally, the substrate comprises a Hydrolyzed Collagen Powder, e.g. made from a type I (e.g. bovine) collagen (e.g. sold under the tradename HYCOL® or Stimulen™)

Optionally, the substrate comprises a non-hydrolyzed type 1 (e.g. bovine) native collagen (e.g. sold under the tradename Collatek®), e.g. which retains substantial native triple helical protein structure of the collagen.

Optionally, the substrate comprises microfibrillar collagen (e.g. microfibrillar collagen type 1), e.g. provided as a sheet or powder (e.g. sold under the tradename CuraVance™).

Optionally, the substrate comprises a hydrolyzed collagen gel (e.g. sold under the tradename Stimulen™).

Optionally, the substrate comprises a non-hydrolyzed (e.g., type 1 such as bovine type 1) native collagen sheet (e.g. sold under the tradename SkinTemp® II).

Optionally, the substrate comprises native (type I) collagen in which the triple helix formation is intact.

Optionally, the substrate comprises denatured or reconstituted collagen, e.g. as a gelatin.

Optionally, the substrate comprises a hydrocolloids (e.g. sold under the tradenames DuoDerm®, ExuDerm®, OriDerm® hydrocolloid, 3M Tegasorb® hydrocolloid dressings) such as formulated as an occlusive or semiocclusive dressing that is composed of gelatin, pectin, and/or carboxymethylcellulose.

Optionally, the substrate comprises lyophilized type I native collagen (e.g. extracted from horse or other equine or other mammal, such as from a tendon such as the flexor tendon). An example of such is sold under the tradename BIOPAD®.

Optionally, the substrate comprises type I native collagen (e.g. 100% native) with native triple-helix, for example, obtained from bovine. An example of such is sold under the tradename Puracol®. Optionally, the substrate further comprises silver (e.g. in the form of Ag+). An example of such is sold under the tradename Puracol Plus®.

Optionally, the substrate comprises a fibrin sheet (e.g. a sheet of polymerized fibrin). Optionally, the substrate further comprises leukocytes and/or platelets (e.g. isolated from blood). The substrate is optionally autologous or allogenic to the wound (i.e. from the same subject or a subject of the same species). An example of a patch comprising a fibrin sheet, leukocytes, and platelets is sold under the tradename 3C Patch®.

Optionally, the substrate comprises a collagen matrix and further comprises one or both of and carboxyl methylcellulose (e.g. configured to absorb excess wound exudate) and sodium alginate (e.g. configured to absorb excess wound exudate). Optionally, the substrate further comprises ethylenediamine-tetraacetic acid (EDTA, e.g. configured to remove zinc to inhibit MMP activity). An example of a collagen matrix comprising carboxyl methylcellulose, sodium alginate, and EDTA is sold under the tradename BIOSTEP®.

Optionally, the substrate comprises a flowable (e.g. liquid) fibrin composition or fibrinogen composition. The substrate can be created, e.g. in vitro or in situ, for example, by mixing fibrinogen and Thrombin to cause thrombin-catalyzed conversion of fibrinogen to fibrin. A two-container system comprising independent fibrinogen and thrombin is sold under the tradename EVICEL®.

Optionally, the substrate comprises bovine type I collagen. Examples of such include Gentell® Collagen Wound Dressings and Particles available at https://www.gentell.com/products/.

Optionally, the substrate is a polyether (e.g. reticulated polyether), polyurethane, and charcoal foam, for example, the foam used in the INVIA® FOAM DRESSING KIT.

Optionally, the substrate comprises a mixture of ECM proteins, e.g. one or more collagens such as Types I, III, and IV, and optionally additional ECM proteins or other ECM components such as one or more of fibronectin, elastin, and a glycosaminoglycan(s) (‘GAG’s, e.g. hyaluronan). Optionally, additional components are included such as GAGs, hyaluronic acid, and laminin. Such a substrate is optionally obtained from foresomach tissue (e.g. from an ovine source). Such a substrate is optionally provided as a fenestrated sheet. Optionally, the substrate further comprises ionic silver. An example of such is the ENDOFORM® Dermal Template and Antimicrobial Dermal Template.

Optionally, the substrate comprises a collagen matrix, optionally with an with an intact ECM. For example, the substrate can comprise ECM associated components such as elastin, fibronectin, glycosaminoglycans, and laminin. An example of such is the Myriad Matrix®.

Optionally, the substrate comprises Type I collagen (e.g. from tendon) and chondroitin-6-sulfate. Optionally, the substrate further comprises silicon. An example of such is the Integra® Bilayer Matrix Wound dressing.

Optionally, the substrate comprises Type I collagen and Type III collagen, wherein the ratio (w/w) of Type I to Type III collagen is at least 5:1 or at least 10:1. Such a substrate is optionally of bovine origin. An example of such is the Maidenbio® collagen sheets and pellets (e.g. CP-10).

Any of the examples taught hereinabove can be provided as a sheet, pellets, or powder, or as a sheet, pellets, or powder mixed with a diluent (e.g. water), e.g. to provide a collagen dispersion. Optionally, the substrate comprises a combination of a sheet (e.g. collagen sheet) and pellets (e.g. collagen pellets).

### Protein Combinations

Optionally the substrate comprises collagen in combination with one or more structural proteins other than collagen, e.g. fibronectin, fibrin, laminin, elastin, gelatins, and mixtures thereof. Optionally, the collagen content of the substrate is greater than any of the one or more structural proteins other than collagen (e.g. collagen may be provided in the substrate in a majority amount).

### Biodegradability

The substrate as a whole or optional ECM protein thereof can be configured to be biodegrade by the host (e.g. a host wound site). For example, the ECM protein or substrate can be selected such that it degrades from proteolytic enzymes such as collagenase and other enzymes found in wounds. Optionally, the ECM protein is selected such that its degradation products induce chemotaxis of human fibroblasts (e.g. optional collagen types I-III).

The substrate can be configured to biodegrade (e.g. when placed in a wound cavity) over the a period of time. The period of time can be, for example, at least 3 days, at least 7 days, at least 2 weeks, or at least 30 days. The period of time (e.g. any of the aforementioned times) can be less than 180 days or less than 10 years or less than 20 years.

Additionally or alternatively, the substrate is resorbable. For example, collagen sheets or particles, or other resorbable substrate, can be placed in a wound cavity and be gradually broken down and/or resorbed by the patient. Substrate (e.g. collagen) may be configured such that it is incorporated into the healing wound (e.g. skin or lower layers). The incorporation can comprise a process of granulation tissue infiltrating the substrate. The substate (e.g. collagen) can be configured to be degraded with normal matrix turnover in the wound (also called remolding).

### Crosslinking

Optionally, the ECM protein (collagen) is cross-linked (e.g. covalently) with itself or another moiety. In some embodiments, the cross-linked ECM protein (collagen) provides the substrate in the form of a sponge, hydrogel, sheet, film, or membrane. The selection of crosslinked moieties and degree of crosslinking can, for example, be tailored to achieve a desired physical or chemical property strength and rate of degradation in a wound site. Crosslinking can, for example, impart proteolytic resistance.

Examples of useful crosslinking agents (e.g. for collagen) include Acetic acid, EDC (1-Ethyl-3-(3-dimethylaminopropyl)carbodiimide) in the presence of NHS (N-hydroxysuccinimide), and Glutaraldehyde (GTA).

EMC proteins (e.g. Collagen) can be crosslinked using methods generally known in the art, such as by heat, radiation, or using conventional chemical crosslinking agents such as, for example, aldehydes, carbodiimides, epoxides, or imidazoles. Covalent crosslinking can be achieved by many specific techniques with the general categories being chemical, radiation and dehydrothermal methods. Optionally, the collagen is cross-linked by gamma irradiation, e.g. in which collagen is exposed to gamma irradiation of at least 0.20 M rads such as a range of 0.25 M rads to 2.0 M rads or in the range of 6 kGy and 8 kGy.

Optionally a crosslinked ECM protein is provided by treating collagen to increase the level of covalent bonding among individual collagen molecules. The Crosslinks are optionally in the form of disulfide bonds. The inter-chain disulfide bonds are one type of cross-linking and can optionally be used to provide additional molecular stability. An increase in cross-linked type collagen may result in a longer persistence of the collagen material when used in a subject as compared to lesser or no crosslinked materials. Cross-linked collagenous tissue, in some embodiments, as compared to lesser or no cross-linked tissue may have one or more of the following characteristics: increased tensile or structural strength, increased resistance to enzymatic degradation, reduced antigenicity and reduced immunogenicity.

Optionally, the substrate comprises crosslinked type I collagen and/or crosslinked type III collagen. Optionally the Type I collagen is crosslinked and Type III collagen is crosslinked. Optionally the Type I is non-crosslinked and Type III collagen is noncrosslinked. Optionally the Type I collagen is crosslinked and Type III collagen is noncrosslinked. Optionally the Type I collagen is non-crosslinked and Type III collagen is crosslinked.

Optionally, the substrate comprises non-crosslinked or partially crosslinked collagen (e.g. as described by US20060159731) or crosslinked collagen (e.g. as described by US20060159731).

Optionally, the substrate comprises a crosslinked network of collagen provided as a hydrogel. For example, the hydrogel may be formed as the collagen is fibrillated, or it may form a network after fibrillation. Optionally, the process of fibrillating the collagen also forms gel-like network. Once formed, the fibrillated collagen network may optionally be further stabilized by incorporating molecules with di-, tri-, or multifunctional reactive groups that include chromium, amines, carboxylic acids, sulfates, sulfites, sulfonates, aldehydes, hydrazides, sulfhydryls, diazarines, aryl-, azides, acrylates, epoxides, or phenols.

Optionally, crosslinking is used to create chemical bonds between collagen molecules. A crosslinking reaction can use used to stabilize the collagen structure and in some cases form a network between collagen molecules. Any suitable crosslinking agent known in the art can be used including, without limitation, mineral salts such as those based on chromium, formaldehyde, hexamethylene diisocyanate, glutaraldehyde, polyepoxy compounds, gamma irradiation, and ultraviolet irradiation with riboflavin. The crosslinking can be performed by any known method; see, e.g., Bailey et al., Radiat. Res. 22:606-621 (1964); Housley et al., Biochem. Biophys. Res. Commun. 67: 824-830 (1975); Siegel, Proc. Natl. Acad. Sci. U.S.A. 71: 4826-4830 (1974); Mechanic et al., Biochem. Biophys. Res. Commun. 45: 644-653 (1971); Mechanic et al., Biochem. Biophys. Res. Commun. 41: 1597-1604 (1970); and Shoshan et al., Biochim. Biophys. Acta 154: 261-263 (1968). Useful Crosslinkers include isocyantes, carbodiimide, poly(aldehyde), poly(azyridine), mineral salts, poly(epoxies), enzymes, thiirane, phenolics, novolac, resole as well as other compounds that have chemistries that react with amino acid side chains such as lysine, arginine, aspartic acid, glutamic acid, hydroxylproline, or hydroxylysine.

Optionally, a collagen is chemically modified to promote chemical and/or physical crosslinking between collagen fibrils. Chemical crosslinking may be made using reactive groups such as lysine, glutamic acid, and hydroxyl groups on the collagen molecule project from collagen's rod-like fibril structure. Crosslinking that involves these groups may be used to prevent the collagen molecules from sliding past each other under stress and thus increases the mechanical strength of the collagen fibers. Examples of chemical crosslinking reactions include but are not limited to reactions with the ε-amino group of lysine, or reaction with carboxyl groups of the collagen molecule. Enzymes such as transglutaminase may also be used to generate crosslinks between glutamic acid and lysine to form a stable γ-glutamyl-lysine crosslink. Inducing crosslinking between functional groups of neighboring collagen molecules is known in the art

Optionally, the substrate is made by precipitating collagen in solution (e.g. using a buffer) and then centrifuging to obtain a collagen pellet that is then diluted water to a desired concentration, and then the collagen is crosslinked to create a substantially insoluble material.

### Collagen Manufacture

Optionally, a network of collagen that provides the substrate is produced in vitro as materials for biomedical applications. For example, monomers of the collagen triple helix are extracted from animal tissue, such as bovine dermis, either by acid treatment or treatment with protein degrading enzymes such as pepsin, to solubilize collagen from the tissue. Once purified, these solubilized collagens (often mixtures of monomers, dimers and trimers of the collagen triple helix) can be fibrillated into fibrils through a pH shift in aqueous buffers. Under the right conditions, the collagen monomers self-assemble into fibrils, and depending on their source and how they were isolated, the fibrils can physically crosslink to form a solid hydrogel. In addition, recombinant collagens and collagen-like proteins have been shown to fibrillate in vitro through similar adjustments in pH and salt concentration. Examples of such products for medical applications include a biodegradable collagen matrix made from a collagen slurry that self-assembles into macroscopic collagen fibers, U.S. Pat. No. 9,539,363, and an organized array of collagen fibrils produced by use of external guidance structures or internal templates and the application of tension, U.S. Pat. No. 9,518,106.

### Additives

The substrate can optionally comprise one or more additives, e.g. in addition to an ECM (e.g. collagen) protein. Examples include silver (e.g. in the form of Ag+ such as silver chloride)), alginate, oxidized regenerated cellulose, and protease inhibitors.

Optionally, the substrate comprises a collagen-based matrix (e.g. Type I, II, or III) and a compound selected from the consisting of collagen types IV and V, fibronectin, laminin, hyaluronate, a proteoglycan, epidermal growth factor, platelet derived growth factor, angiogenesis factor, antibiotic, antifungal agent, a spermicidal agent, a hormone, an enzyme and an enzyme inhibitor.

Optionally, the substrate comprises a mixture of collagen and glycosaminoglycan (e.g. covalently linked together or not covalently linked together), e.g. as disclosed by U.S. Pat. Nos. 4,808,570 and 7,993,679B2. Optionally, the mixture is an aqueous dispersion of collagen and a glycosaminoglycan. Optionally, the glycosaminoglycan is a member selected from the group consisting of chondroitin 6-sulfate, chondroitin 4-sulfate, heparin, heparin sulfate, keratin sulfate, dermatan sulfate, and combinations thereof. Optionally sources of proteoglycans include bovine and human cartilage and synovial fluid, nasal septum and sternal cartilage, and skin. Optionally sources for glycoproteins also include EHS tumor, bovine and human kidneys, cartilage, bone and placenta (e.g. insoluble amnion, the soluble amnion, or the chorion) as well as bovine and human blood. Typical sources of hyaluronate include rooster comb and bovine vitreous.

Optionally, the substrate comprises a mixture of collagen and hyaluronate (e.g. mixed with glycosaminoglycan).

Optionally, the substrate comprises a mixture of collagen and one or more of a glycosaminoglycan, a glycoprotein, a structural protein or a growth factor which are optionally crosslinked with the collagen. Suitable glycoproteins include, but are not limited to, fibronectin, laminin and chondronectin. An example of a suitable structural protein is elastin. Growth factors include but are not limited to, epidermal growth factor (EGF), platelet derived growth factor (PDGF), vascular endothelial growth factor (VEGF), fibroblast growth factor (FGF), nerve growth factor (NGF), bone morphogenetic protein (BMP), and the like

Optionally, the substrate comprises an ECM compound (e.g. collagen) and at least one bioactive molecule effective to enhance wound healing, e.g. one or more of growth factors, anti-inflammatory agents, wound healing agents, anti-scarring agents, antimicrobial agents, cell adhesion peptides, tissue generation modulating cells, nucleic acids, nucleic acid analogues, proteins, peptides, amino acids, ceramic, and combinations thereof.

### Sterility and Purity

Optionally, the ECM protein (e.g. collagen) of the substrate is decontaminated or sterilized without significantly affecting the physiological properties of the ECM protein (e.g. collagen) using gamma or electron-beam radiation. The substrate can be irradiated under conditions that inactivate any pathogenic microorganisms, viruses, and polynucleotide fragments thereof, DNA or RNA, whether single or double stranded present within the substrate.

Optionally, a substrate is substantially free of contaminates. For example, a collagen substrate can be provided which does not contain hair, hair follicle(s), or fat(s) of an animal that naturally expresses the collagen molecules. Optionally, the substrate is substantially free (or contains less than 1, 2, 3, 4, 5, 6, 7, 8, 9, or 10% by weight) of actin, keratin, elastin, fibrin, albumin, globulin, mucin, mucinoids, noncollagen structural proteins, and/or noncollagen nonstructural proteins. Optionally, the substrate substantially free of other collagenous proteins, carbohydrates, nucleic acids, or lipids, or immunogens, antigens, or allergens.

Optionally, the collagen is substantially free of one or more substances selected from gelatin, live cells and/or cellular debris (e.g. is dellularized).

Optionally, the substrate comprises purified collagen purified or a concentration of collagen that allows the collagen to fibrils.

Optionally, the substrate comprises 25, 30, 40, 50, 60, 70, 80, 90, 95 or 99% by weight collagen based on its total protein content or based on its total weight.

### Dried ECM Protein

Optionally, the substrate comprises a dehydrated or dewatered ECM protein (e.g. collagen). “Dehydrating” or “dewatering” is a process of removing water from a mixture containing collagen and water, such as an aqueous solution, suspension, gel, or hydrogel containing collagen. Water may be removed by filtration, evaporation, freeze-drying, solvent exchange, vacuum-drying, convection-drying, heating, irradiating or microwaving, or by other known methods for removing water. In addition, chemical crosslinking of collagen is known to remove bound water from collagen by consuming hydrophilic amino acid residues such as lysine, arginine, and hydroxylysine among others.

Optionally, the substrate may be provided in the form of a sheet (e.g. solid sheet or semi-solid sheet or gel sheet) or powder comprising collagen. For example, a collagen sheet can be provided by dehydrating (e.g. lyophilizing) or partially dehydrating a solution or suspension of collagen (e.g. soluble and/or insoluble collagen) and optionally compressing the resulting sheet.

Optionally, the substrate is dried to an amount such that the substrate absorbs 5-20 times its weight in wound drainage.

### Product

One aspect of the invention provides a product comprising an MEF or other extract (e.g. a bivalve extract or an echinoderm extract). The product can be the MEF formulation alone (with or without a container), or can be a combination of the MEF (or other extract) and one or both of a dressing and a substrate. Another aspect of the invention provides a product comprising a substrate and one or both of a dressing and an extract (e.g. MEF).

A product of the present invention can be provided in the form of a composition (e.g. extract), substrate, dressing, or a kit. For example, the kit can comprise the two or more components selected from the composition, the dressing, and the substrate.

Optionally, the product comprises a layer comprising the MEF (or other extract) and another layer comprising the dressing.

Optionally, the product comprises a layer comprising the MEF (or other extract) and another layer comprising the substrate.

Optionally, the product comprises a layer comprising a mixture of the MEF (or other extract) and the substrate and another layer comprising the dressing.

Optionally, the product comprises a first layer comprising a substrate (e.g. collagen substrate) configured for contact with a wound, a second layer comprising the dressing, and either the first layer further comprises the MEF (or other extract) or the product comprises a third layer comprising the MEF (or other extract). Optionally, the product comprises the three layers and the second layer is between the first layer and the third layer. Alternatively, the third layer is optionally between the second layer and the first layer. The second layer can be configured to be larger in area than the first layer such that it is in contact with the first layer and a portion of the skin surrounding the wound. The second layer can be configured to adhere to the portion of skin surrounding the wound and is effective to maintain the first layer in contact with the wound. The second layer may optionally be in the shape of a sheet or film (e.g. a perforated dressings e.g. as described in US 2021/0007899). The second layer may optionally have only one side that is adhesive (e.g. the adhesive faces towards the side of the first layer). Optionally the first layer further comprises the substrate (e.g. collagen) in the form a hydrogel or dried substrate.

In alternative embodiments, the invention contemplates a product according to any of the above descriptions, except that the MEF is substituted by any extract (e.g. a marine extract such as a bivalve extract, an echinoderm extract).

Additional optional configurations of the product will now be described.

### Dressings

Kits and products can optionally comprise a dressing in combination with one or both of an extract (e.g. MEF such as bivalve extract and/or echinoderm extract) and a substrate. Similarly, methods can optionally comprise the application of a dressing over one or both of an extract and substrate.

Any dressing taught herein can comprises a sheet comprising a plurality of channels (also called perforations). The channels can be configured to allow fluid exchange between the proximal and distal side of the dressing.

Any dressing taught herein can be provided or used with MEF (or other extract) alone, with substrate alone, or with the MEF (or other extract)-substrate combination, securing the substrate and protecting the wound. Preferred are perforated dressings described in US 2021/0007899. Examples of preferred dressing are dressings comprising a sheet with a moisture vapor permeability (in a nonperforated area) of at least 100 gms/m²/24 hours, but preferably at least 500 gms/m²/24 hours, and more preferably at least 1000 gms/m²/24 hours, and more preferably still at least 2000 gms/m²/24 hours, and yet most preferably between 2,500 and 6,000 gms/m²/24 hours (e.g. when measured at 37° C. and 100% to 10% relative humidity).

Any dressing taught herein can be configured as a tape (e.g. having a thickness of less than 3 mm, less than 2 mm, or less than 1 mm).

Dressings of the present invention can be sized to fit the wound. Generally, the wound itself is to be completely covered and extending at the perimeter to adhere to healthy skin (or less damaged skin).

The capacity of the dressing to aid prolonged immobilization of the substrate is dependent upon having a dressing that can remain applied to the skin for one or two or three weeks. It has been discovered that “double protection” of granulation by the substrate and by dressing results in a remarkable improvement of healing and the quality of the skin post healing.

The dressing can optionally be provided as a sheet having adhesive on a side thereof. Optionally, MEF or MEF-substrate combination is provided with a first sized area and the dressing has an area larger than the first area such that a portion of the dressing contacts the MEF or MEF-substrate combination and a portion contacts the skin surrounding the wound, for example, to adhere to the wound via adhesive.

Optionally the dressing has a high moisture vapor permeability. For example, the dressing can have a moisture vapor permeability greater than 2,500 gms/m²/24 hours when measured at 37° C. and 100% to 10% relative humidity. For example, nonwoven rayon with a sheet of a thickness of between 0.05 and 0.25 mm can be configured to provide such moisture vapor permeability. Examples such dressings and configurations with high moisture vapor permeability are taught in US 2021/0007899.

Optionally the dressing comprises a sheet and an adhesive, wherein the adhesive is adhered to the sheet, and wherein the sheet has channels. Optionally, the channels comprise two different classes of channels (e.g. liquid channels and air channels). Examples such dressings are taught in US 2021/0007899.

Optionally, the dressing is any dressing disclosed in US 2021/0007899.

As taught herein such a dressing can optionally contain perforations (also called channels) and wherein the dressing is sized to cover a wound and the substrate is sized to insert into the wound

### Kits

In one embodiment, the invention provides a kit comprising the MEF (or other extract) and one or both of a substrate and a dressing. The kit may comprise a single container that contains a substrate (e.g. powder, particle, gel, or flowable form) and optionally a physiologically acceptable fluid. The container may itself be a syringe, pipette, or other such like apparatus, from which substrate (e.g. collagen) particles or flowable form may be applied to, for example, a wound area or other target site. Optionally, the single container may contain dry collagen particles, which may or may not require pre-wetting before use. The collagen particles may optionally comprise GAG.

### Method of Uses

According to some embodiments, a product can be administered to a subject to treat a condition. The various embodiments of the present invention (e.g., MEF; MEF (or other extract)+substrate and/or dressing; products, and/or kits taught herein) can be used to treat any condition. Examples of wounds that can be treated include pressure ulcers, surgical wounds, draining wounds, venous ulcers, diabetic ulcers, burns, venous stasis ulcers, abrasions, partial- and full-thickness burns, post-laser surgical injuries, lacerations, dehisced surgical incisions, donor (e.g. graft) sites, and dehisced wounds.

It has also been discovered through empirical studies by Inventor, that recalcitrant wounds (discussed elsewhere herein) and chronic wound conditions from certain genetic diseases can be treated to obtain remarkable healing. Epidermolysis bullosa, Pemphigus autoimmune diseases such as Pemphigus vulgaris, Pemphigus IgA, Paraneoplastic pemphigus, and Pemphigus foliaceus, Pemphigoid diseases such as Bullous pemphigoid and Pemphigoid gestationis, IgA Mediated Bullous Dermatoses such as Dermatitis herpetiformi and Linear IgA disease are examples of medical conditions that can be treated according to the present invention with remarkable results.

A method of the invention optionally comprises the step of topically applying, to a subject in need thereof (e.g. to the surface of the skin having a skin condition), a composition taught herein or product taught herein (e.g. MEF; or MEF+substrate; or MEF+substrate+dressing; or substrate+one or both of an extract and a dressing).

Optionally, the condition is a wound cavity that is at least 0.5 cm deep (measured from the surface of the skin), for example, at least 1 cm deep, at least 2 cm deep, at least 3 cm deep, or at least 5 cm deep. Optionally any of such wounds are no more than 8 cm deep. For example, for gluteal-sacral wounds, a treated wound could be a few inches deep. Optionally, the substrate (e.g. pad, particles, or gel, optionally formed from collagen) comprises a volume that fills at least 70%, 80%, or 90% of the wound cavity.

Optionally, the condition is full thickness wounds which reaches all of the way to the bone.

A wound at any stage can be treated by an embodiment of the present invention. However, the different embodiments of the present invention can offer advantage based upon the severity of the wound. For the sake of discussion, Inventor has staged wounds according to the anatomical features involved as set forth in Table 1.

TABLE 1

| | | Example |
|---|---|---|
| | | Embodiment for |
| Stage | Anatomical damage | treatment |
| Stage 1 | Epidermal damage to the basal cell | MEF or |
| | layer | MEF + Dressing |
| Stage 2 | Damage through the basal cell layer | MEF or |
| | into the papillary dermis | MEF + Dressing |
| Stage 3 - | Damage through the papillary dermis | MEF |
| superficial | into the upper reticular dermis | MEF + Dressing, |
| | | or MEF + Substrate* |
| Stage 3 - | Damage through the papillary dermis | MEF |
| | deep into the reticular dermis | MEF + Dressing |
| | | MEF + Substrate* |
| Stage 3 - | Damage through the reticular dermis | MEF + Substrate |
| deep | into the superficial subcutaneous (fat) | MEF + Substrate + |
| | layer | Dressing |
| Stage 4 - | Damage through the subcutaneous | MEF + Substrate |
| superficial | layer to the fascia/muscle | MEF + Substrate + |
| | | Dressing |
| Stage 4 | Damage into muscle to periosteum of | MEF + Substrate |
| | the bone | MEF + Substrate** + |
| | | Dressing |
| Stage 4 - | Damage through the periosteum into | MEF + Substrate |
| deep | bone (deep stage 4). | MEF + Substrate** + |
| | | Dressing |

*Substrate optimally sized to be smaller than 8³ mm
**Substrate sized to the wound cavity can be optimal

In alternative embodiments, the present invention contemplates methods in which the MEF in such examples is substituted by any extract (e.g. a marine extract).

### Mild Wounds.

As set forth in Table 1, mild wounds (such as Stage 1-2) can be usefully treated with MEF. In these wounds, the skin is injured only as deeply as the papillary dermis. The addition of substrate to the wound may not be as useful as it is for deeper wounds. Without being bound by theory, Inventor believes that there are compounds in MEF that augment natural pathways leading to wound repair and producing skin of normal, healthy appearance.

MEF can be added daily when as a cream, lotion, etc. or as any formulation taught herein. Mild wounds can also be usefully treated with MEF with a dressing according to the present invention.

### Moderate Wounds.

As set forth in Table 1, moderate wounds (such as Stage 3-4) can be usefully treated with the combination of MEF and substrate; often with MEF and dressing, according to the present invention.

Inventor has discovered that combining MEF with substrate can increase both quality and speed of healing. Without being bound by theory, Inventor believe that substrate can facilitate granulation tissue filling the wound bed with connective tissue and that MEF acts at least at this stage in concert.

### More Serious Wounds.

Inventor has discovered that for wounds, especially deep wounds or wounds affecting a larger area, the addition of a dressing can greatly facilitate maintaining proper positioning of substrate. Having maintained the proper position of the substrate, Inventor has discovered that granulation and re-epithelialization are supported to an extent that wound healing proceeds to the remodeling/maturation stage where substrate is degraded and replaced by new collagen synthesis and extracellular matrix proteins. At completion, aided by MEF, skin regions normal, healthy appearance.

### Clinical Observations

It has long been noticed that under certain medical conditions or for certain injury types, wounds can be partially or entirely recalcitrant to healing. Through personal observation and exploratory medical treatment, Inventor has identified the proliferative phase of wound healing as a critical target for therapeutic treatment. For example, Inventor believes that typical wound care can impede wound healing by interfering with formation of granulation tissue. Such care includes periodic dressing change and washing of the uncovered wound. Moreover, deep wounds or large areas are especially prone to fail at the proliferative phase—especially the migration and colonization of granulation tissue. However, according to the present invention, the proliferation phase is supported by one or more of by substrate, MEF, and dressing. Moreover, based on empirical data, Inventor believes that the combination (MEF, dressing, and/or substrate) enhances healing by an unexplained action in concert. Furthermore, because the substrate/dressing embodiment can remain in place for one to three weeks or more, disruption by dressing changes can be avoided. While the mechanism of MEF is being investigated, Inventor has observed that when used with substrate or substrate and dressing according to the present invention, the healed wound has an improved appearance, e.g. relative to a healed wound treated without the MEF.

The publications cited herein are incorporated by reference for the cited subject matter.

### EXAMPLES

### Example 1 Use of Products to Treat Wounds

Applicant has regularly examined subjects with wounds that were recalcitrant to healing. Such wounds were often observed in aged subjects (e.g. 70 year age or older) or diabetic individuals or subjects with chronic insult (e.g. paraplegic, quadriplegic, or non-bed-ridden subjects). In general, the procedure was performed as follows.

A medical history is taken and the wound is cleaned and debrided as appropriate. Wounds are evaluated according to multiple variables and a treatment protocol is selected.

Wounds are generally treated as set forth in Table 1. The substrate is made from collagen. The collagen is formed from native Type I collagen present in a majority amount (>90% type I collagen in this example) with a minor amount of Type III collagen (1-10%) extracted from mammalian tendon.

The substrate collagen was extracted from mammalian tendon (bovine in this example). Specifically, the substrate collagen is obtained from Maidenbio®, in the form of pellets (product CP-10) or sheets (e.g. CD-7070). In the present example, the only ECM component contained in the substrate is Type I collagen (>90%) with a minor amount (<10%) of Type III collagen, derived from bovine tendon, although the present inventor believes that the composition of the substrate may be varied, so long as the substrate provides a medium or scaffolding for infiltrating granulation tissue.

The MEF contains a mixture of bivalve extract and echinoderm extract. The MEF is provided in dehydrated form by lyophilization, although the present inventor believes that other forms of MEF may be used as well.

The bivalve exact is obtained from a scallop (pink scallop in this example).

The echinoderm exact is obtained from a sea cucumber.

Each of the bivalve exact and the echinoderm exact is made by removing the digestive gland and gonads from the remaining portion of the animal, lyophilizing the removed digestive gland tissue and gonad tissue, and then grinding the lyophilized tissue to size.

The final extract is a combination of the ground bivalve tissue and the echinoderm tissue.

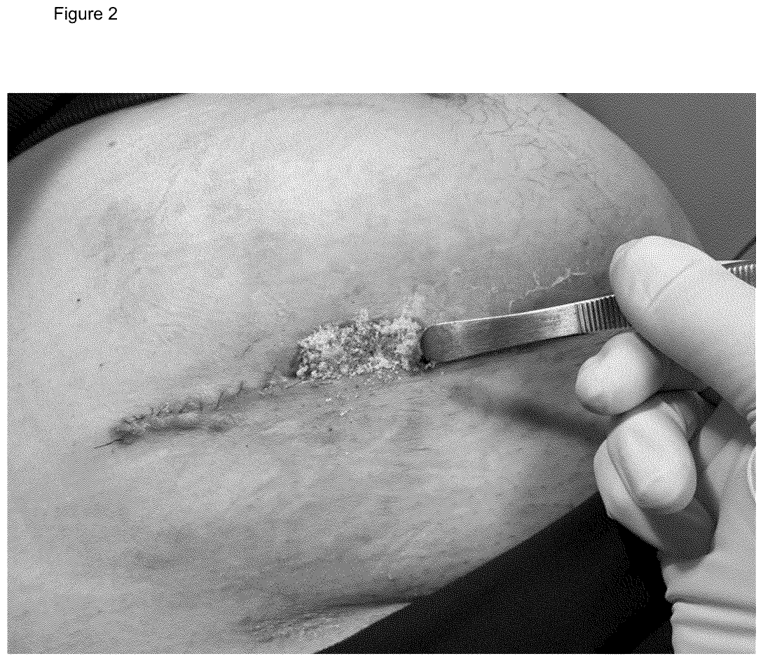

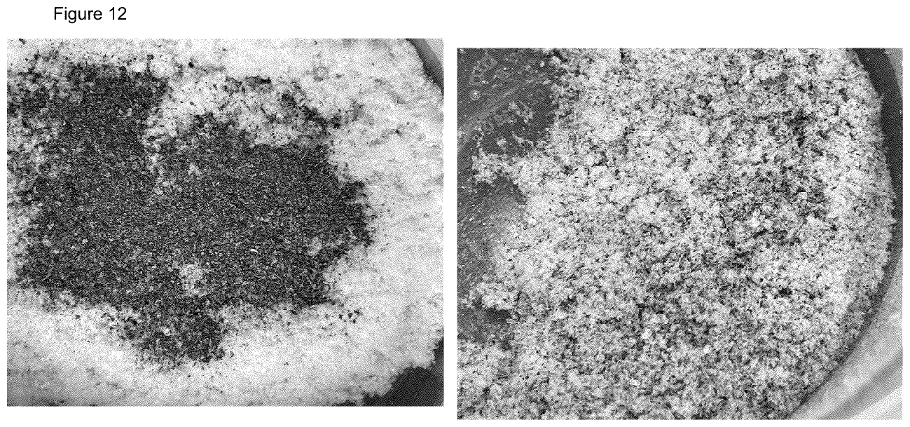

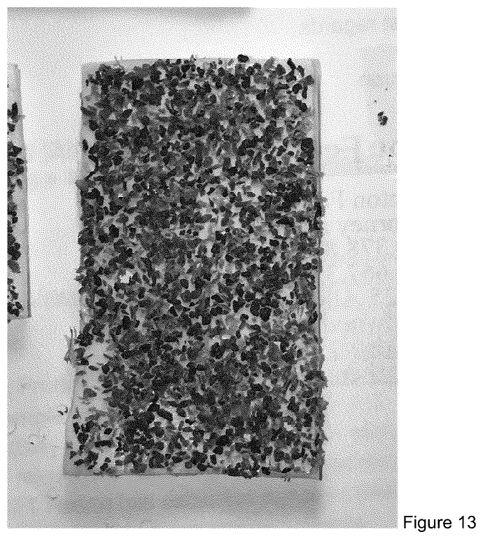

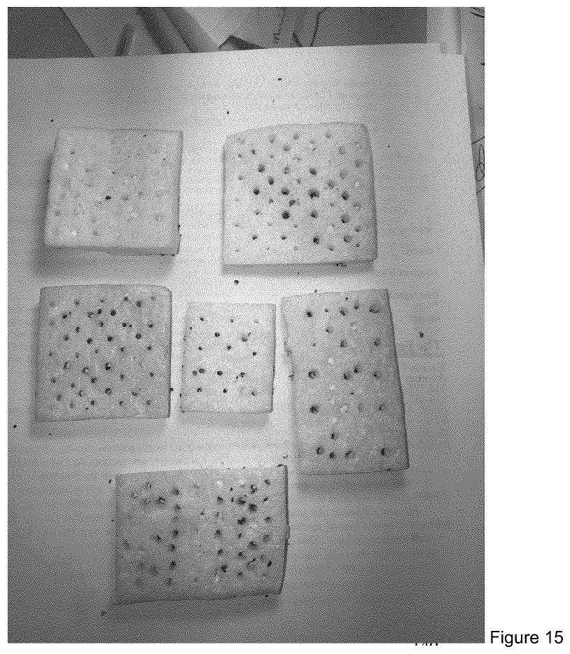

For some deep wounds, substrate is selected and sized wherein a single sheet of collagen fills a majority of the wound cavity. In other cases, the wound cavity is filled with the substrate in pellet form. In the case of substrate sheets (e.g. thick pads), the MEF is layered on the wound-side of the sheet (see FIG. 13) and/or impregnated into the sheet through channels made in the sheet by Applicant (see FIG. 15), before placing the combination of the substrate and MEF in the wound cavity. The substrate sheets can be of any thickness, e.g. 5 mm to 4 cm or even thicker, to completely fill or substantially fill the depth of the wound cavity. In the case of substrate particles, the MEF is either placed in the wound and then covered with the particles, or mixed with the particles (FIG. 12 right panel) before inserting the mixture in the wound cavity (FIG. 2). The volume of particles is selected such that the particles fill or substantially fill the wound cavity.

Applicant performs regular follow-ups as appropriate for the type and severity of wound. Applicant makes the following conclusions from wounds treated according to the instant invention:
1. Generally, these invention-treated recalcitrant wounds heal completely.
2. Said healing can progress temporally in a manner similar to in individuals without co-morbidities treated with standard of care.
3. After healing, the skin generally has an appearance of healthy skin (e.g. hypertrophic scar and keloid or discoloration).
4. Generally, embodiments that contain the MEF of bivalve extract and echinoderm extract demonstrate a remarkable resistance to infection.

### Example 2 Use of Products to Treat Wounds

A 34-year-old woman patient presented to Applicant. She had undergone a 100+ stitch repair of a 30 cm long, 6 cm wide and 3.7 cm deep scar (penetrating down to fascia) from an infected appendicitis surgery at the age of 12. Twelve days after the repair, she had a traumatic dog injury which tore a hole just above the suture line and penetrated 3.7 cm down to fascia. She returned to the clinic three days after the dog injury

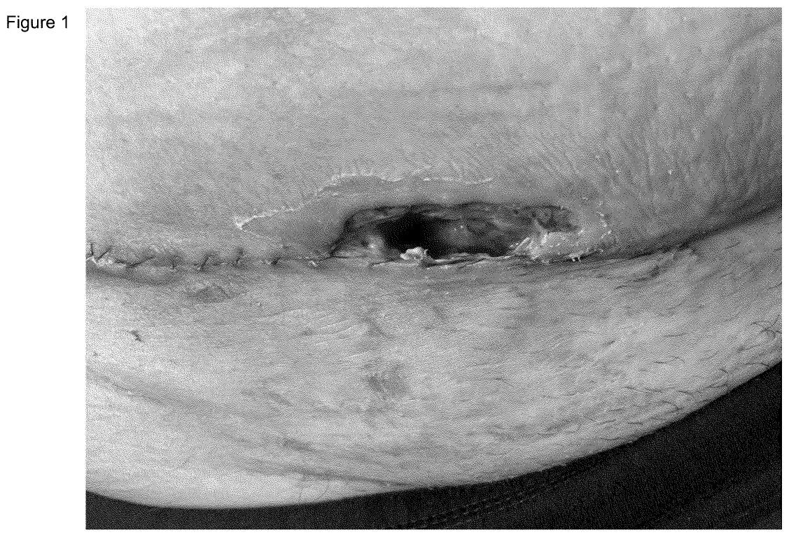

The wound (FIG. 1) was debrided and a wet-to-dry dressing was placed in the cavity.

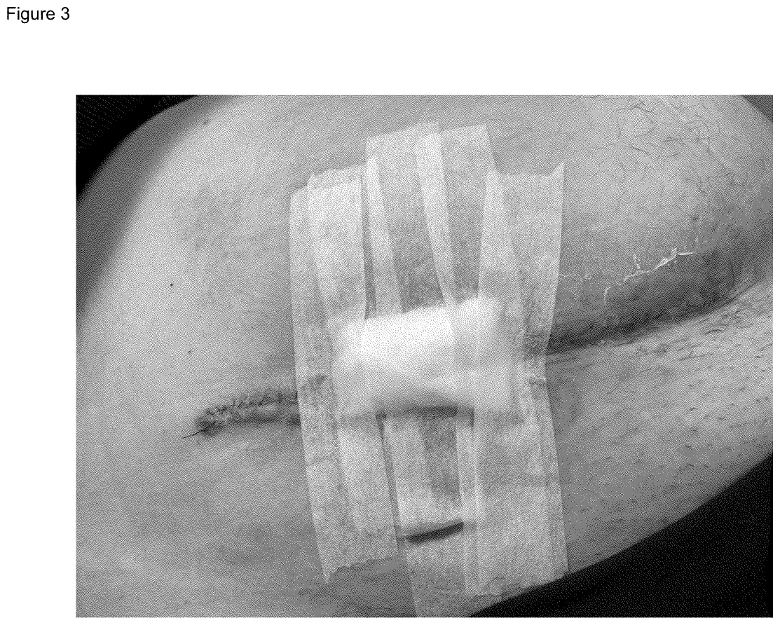

On the next day, the wound was again irrigated and lyophilized MEF was combined with collagen pellets (pellets described in Example 1), mixed, and packed into the wound (FIG. 2). The packed MEF/collagen pellets were dampened and secured with gauze pad secured with paper tape (FIG. 3). The lyophilized MEF contained a lyophilized mixture of bivalve extract and echinoderm extract in the same manner as described in Example 1.

Every 3-4 days the wound was repacked with MEF/collagen pellets, the packing dampened, and a gauze dressing placed and secured with paper tape over it.

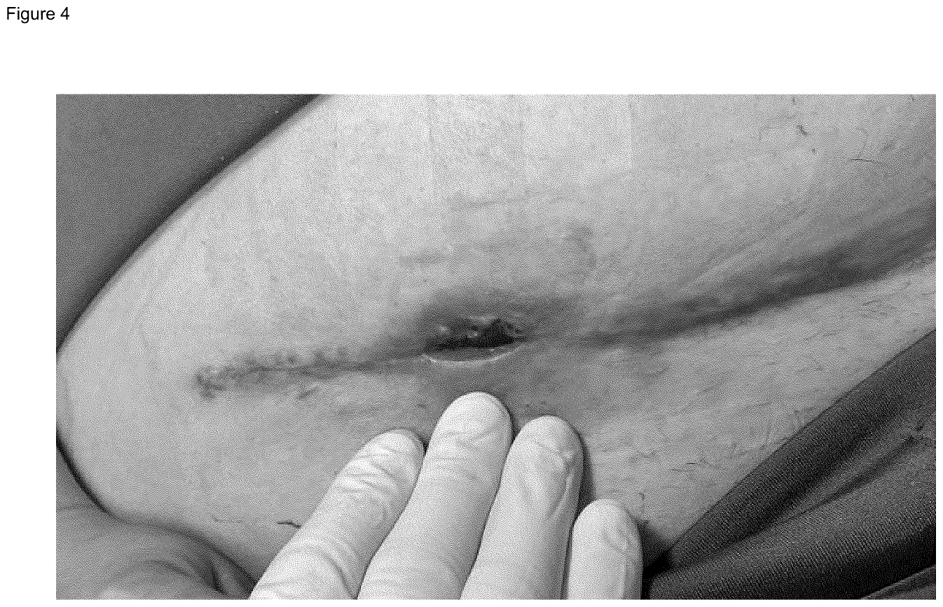

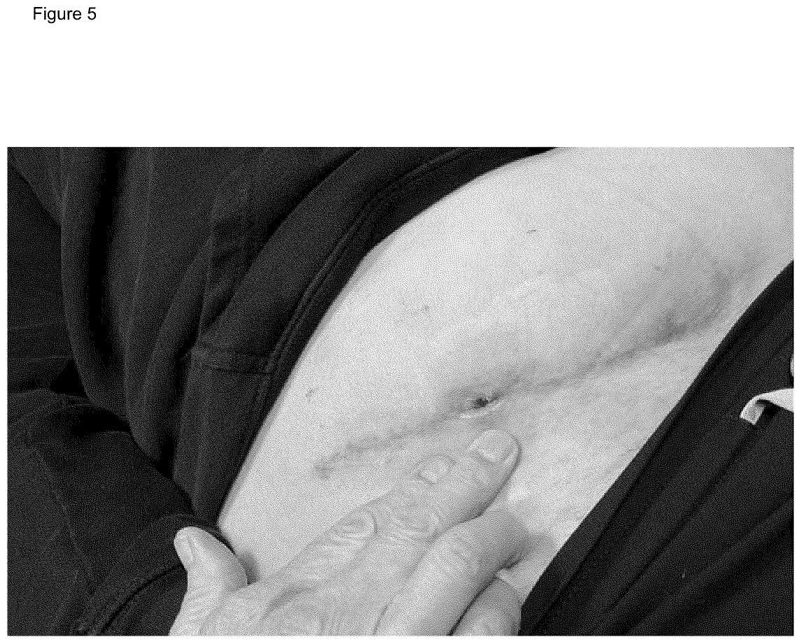

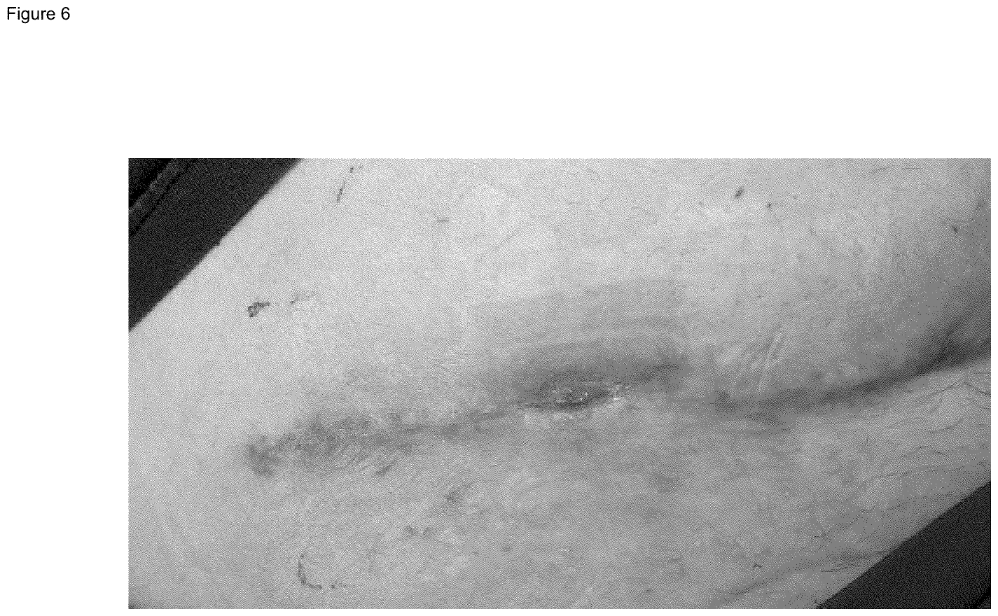

Over the next 3 weeks, the Applicant noted progressive healing at 22 days (FIG. 4), 36 days (FIG. 5), and 40 days (FIG. 6). This wound initially appeared as a wound that would be expected to take three months or more to heal instead. Instead, the wound healed in about half that time. This is especially remarkable considering the extent of inflammation and the nature of a traumatic dog wound and the tremendous amount of scar tissue from the original appendicitis scar.

As can be seen in FIG. 2, the pellets fill the wound to the periphery (see top and bottom of wound), although the left and right sides of the periphery have not yet been filled with pellets by the physician in FIG. 2. Alternatively, a sheet could be perfectly cut to fit the wound shape. If such is difficult to perform, an alternative method involves placing a substrate sheet in the middle of the wound and filling gaps around with pellets to touch the periphery of the wound (as in the top and bottom of the wound shown in FIG. 2).

### Example 3 Use of Products to Treat Wounds

In this example, a 77-year-old man presented six months after Mohs surgery for squamous cell carcinoma. He had long term diabetes and a history of poor healing. The resultant wound was increasingly painful and accompanied by leg swelling and decreasing sensation in the foot. During this 6-month period, the subject saw multiple dermatologists and wound care specialists for numerous wound treatments. Nonetheless, the wound worsened.

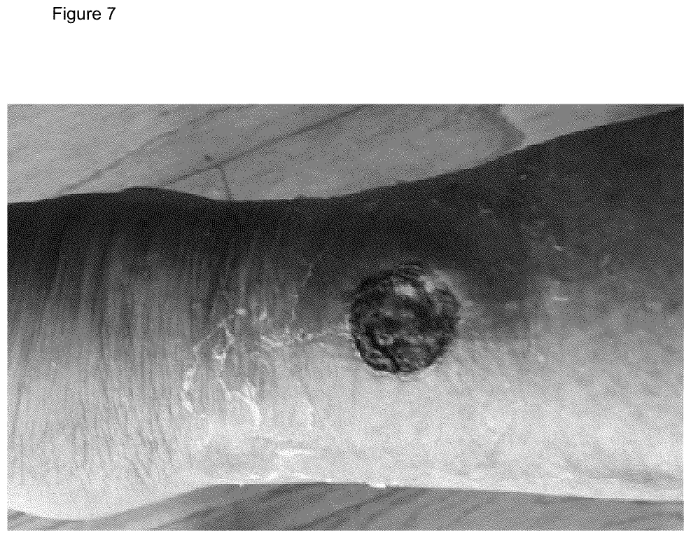

At time of presentation to Applicant-physician, the wound had progressed into the fat layer. There was apparent swelling, venous congestion (purple), necrotic debris, generalized calf and foot swelling, and the subject reported pain all the way to the foot. See FIG. 7.

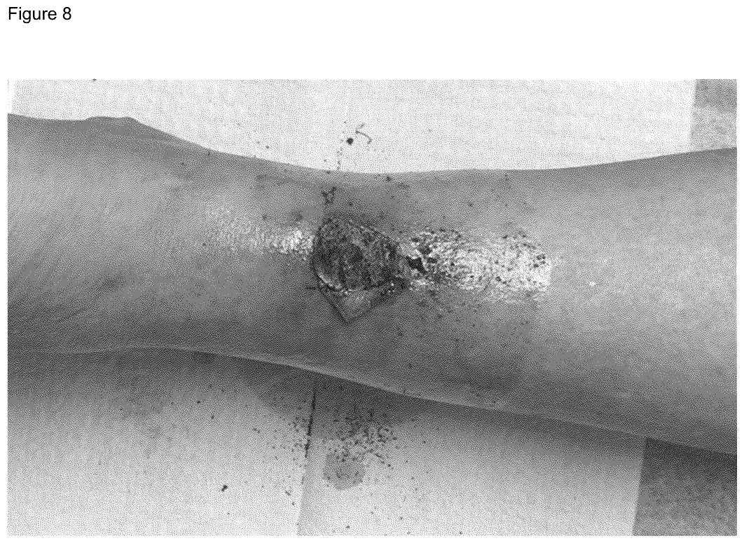

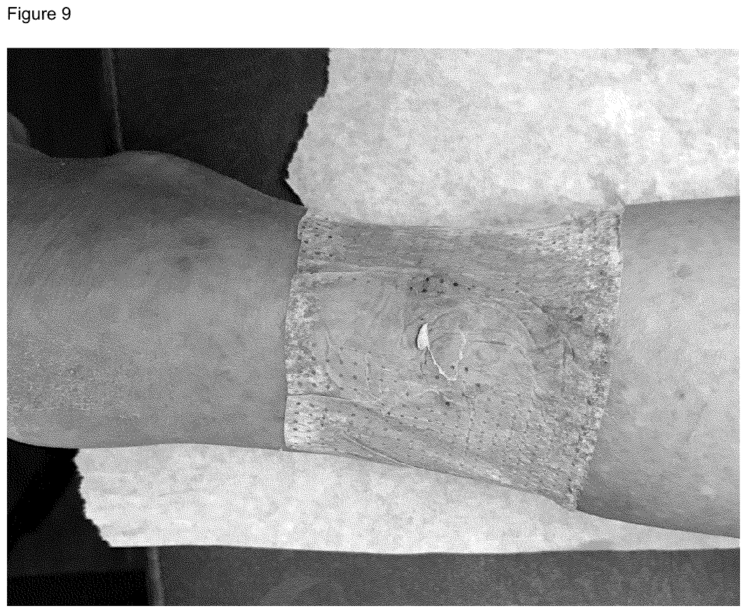

The wound (FIG. 8) was debrided and irrigated. MEF (lyophilized) was sprinkled into the wound and a collagen pad was sized and placed in the wound. Specifically, the MEF contained a lyophilized mixture of bivalve extract and echinoderm extract, prepared in the manner described in Example 1. Next, the wound was covered with a perforated dressings as set forth above. See FIG. 9

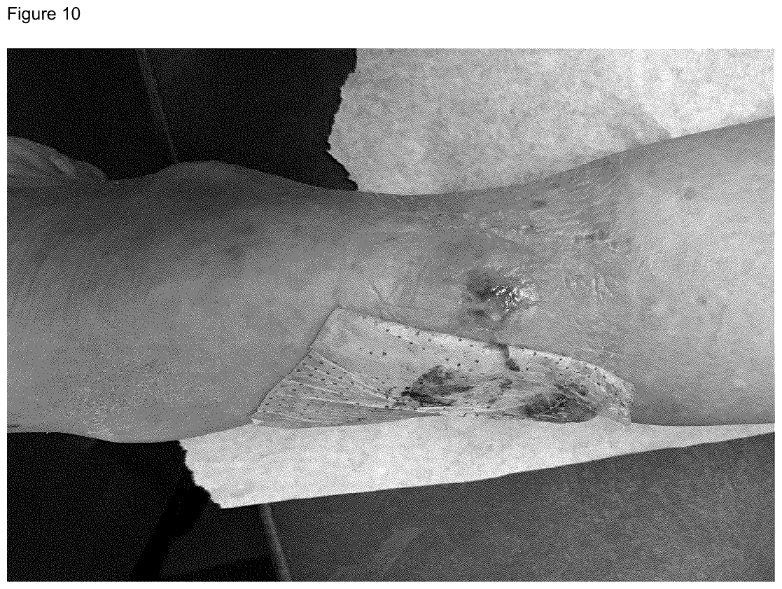

During the next 3 weeks, the patient was instructed to leave the dressing in place and to cleanse the wound through the dressing as needed. At the end of the 3 weeks, the dressing began to fall of the skin, and it was gently removed by Applicant/physician. As seen in FIG. 10, granulation tissue has fully infiltrated the wound and the collagen substrate.

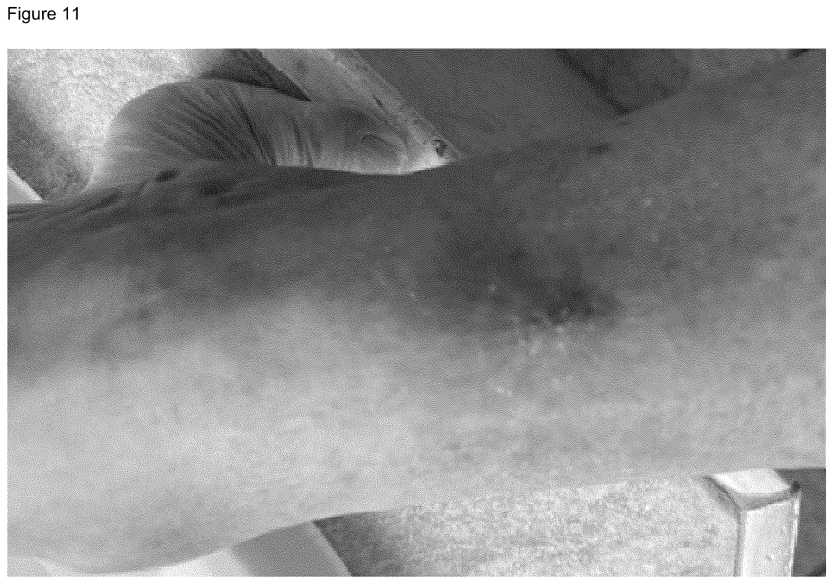

On a follow-up visit, the wound was fully healed. See FIG. 11. This finding is remarkable, especially considering that the wound had been recalcitrant to treatment according to the skilled artisan and the healing was so dramatic.

### Example 4 Products Comprising Dried Form of Extract

Products containing MEF (e.g. as described in the examples above) may be provided in dehydrated form, such as by lyophilization. The dried form increases shelf life of the MEF, concentrates the MEF, enhances the ability of the MEF to be combined with a substrate, allows the physician to avoid introducing additional moisture into the wound cavity, and prevents escape of MEF from the wound due to inadvertent flowing out of the wound. The dried form of MEF allows the selection of MEF particle size to provide quicker release by the selection of smaller sized particles (see FIG. 14, left product) or extended release by the selection of larger sized particles (see FIG. 14, right product).

Accordingly, while the present invention is not limited to the use of dried extract, it provides a remarkably useful form of the MEF when used on its own, and especially when combined with a substrate.

### Example 5 Products Containing Channeled Substrate

Products containing a substrate (e.g. as described in the examples above) can comprises channels in the substrate, as taught herein.

The use of a channeled substrate allows exchange of liquids and air between the wound and surrounding area, e.g., to prevent excessive moisture build up in the wound cavity.

Moreover, Applicant has discovered that granulation tissue is drawn up into the substrate. The drawing up of granulation tissue can, according to some embodiments, be facilitated by the channels in the dressings taught herein (e.g. having channels and a high moisture vapor permeability in non-perforated regions), thereby providing a protected environment for healing. In such a configuration certain wound healing steps such as granulation proceeds in an accelerated manner. As stated elsewhere herein, the substrate seems to act like scaffolding for granulation tissue which is drawn in by forces greater than simple diffusion and can become incorporated into the healing wound. Without being bound by theory, Applicant believes that this process is responsible for the unexpectedly accelerated healing and qualitatively superior healing

By way of example, FIG. 15 depicts six products having a collagen substrate which have been perforated to form channels.

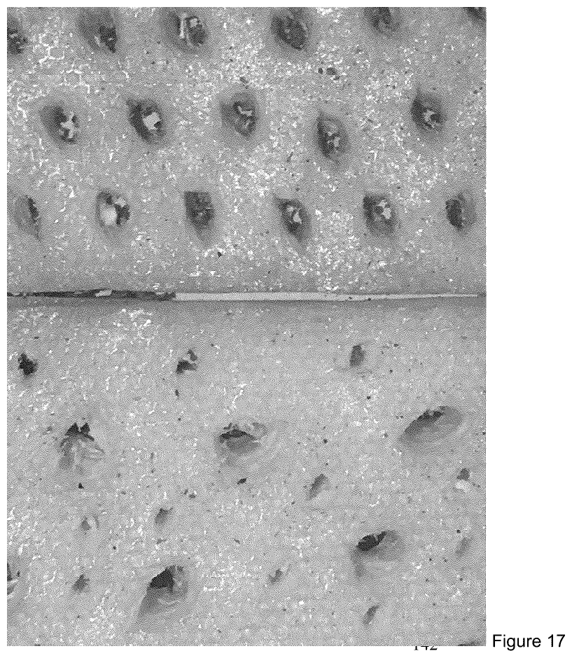

As another example, the substrate can comprise a plurality of channels of different diameters. For example, the substrate can have two sizes of channels (see FIG. 17, top product, noting that the smallest (needle roller punched) sized channels are difficult to see) or three channel sizes (see FIG. 17, bottom product, noting that the smallest (needle roller punched) sized channels are difficult to see).

The top product in FIG. 17 has two channel sizes. The first diameter is 0.5-1 mm and the second diameter is 1-2 mm.

The bottom product in FIG. 17 has three channel sizes. The first diameter is 0.1-0.5 mm, the second diameter is 0.5-1 mm, and the third diameter is 1-2 mm.

The 1-2 mm holes are spaced about 4-6 mm, measured center-to center

Without being bound by theory, the inventor belies that 0.1-0.5 mm channels provides a means for pulling granulosing tissue into a sheet, e.g., provided by enhanced capillary action.

Without being bound by theory, the inventor belies that 0.5-1 mm channels provide an entrance for granulosing tissue and for fluid exchange between the wound and exterior of the sheet.

Without being bound by theory, the inventor belies that 1-2 mm provide for enhanced fluid exchange between the wound and exterior of the sheet.

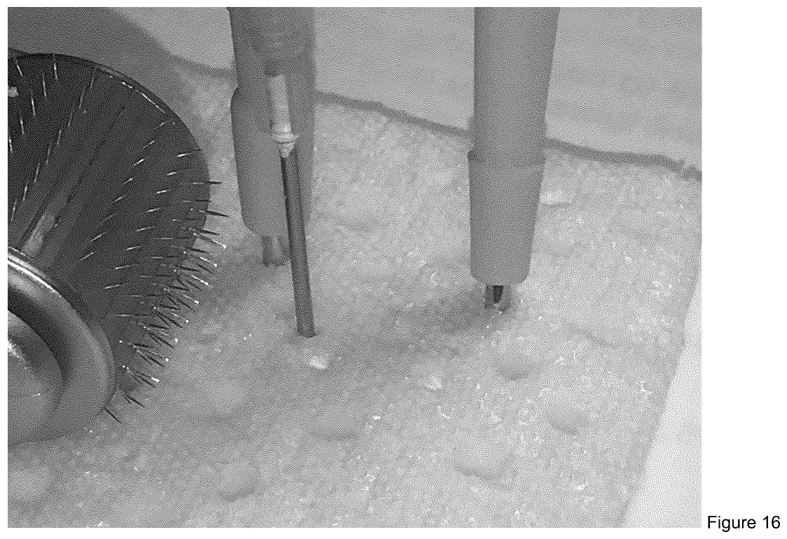

FIG. 16 depicts an example of how the substrate with three sized channels was produced (before elongating and fraying as described below) The smallest sized channels were formed using a needle roller. The middle sized channels were formed using a syringe needle. The largest sized channels were formed using a biopsy punch.

Optionally, the plurality of channels comprises conical channels and/or elliptical channels and/or frayed channels. Such a configuration can be applied to 0.5-1 mm diameter channels and/or diameter 1-2 mm channels.

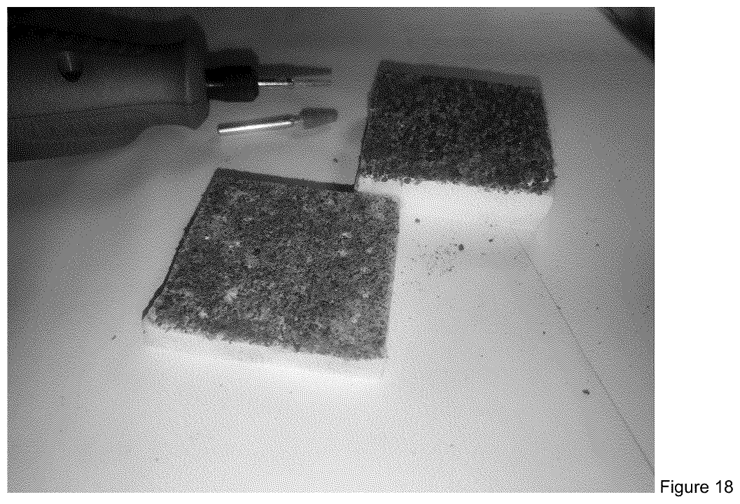

By way of example, inventor used a Dremel® tool with diamond conical fraze bit to make conical channels (see FIG. 18). The channels were elongated along one axis to impart an elliptical shape and then frayed about the inner surface area of the channels to roughen the channels. The inventor believes that the conical, elliptical, and roughened configurations each contribute to an increase in capillary forces, e.g. by increasing surface area.

For the sake of clarity, it is noted that FIG. 18 depicts two products which have been perforated using the above technique. The product in the background of FIG. 18 has a thicker substrate (about 9 mm tall), with MEF layered on the top side and is for thicker wounds and has three size pores as can be seen on the close up (FIG. 17 lower product, showing the back side not covered with MEF). The product in the foreground of FIG. 18 has a thinner substrate (about 4.5 mm tall), with MEF layered on the top side and is for thinner wounds and has two size pores as can be seen on the close up (FIG. 17 upper product, showing the back side not covered with MEF).

### Example 6 Product in the Form of a Kit

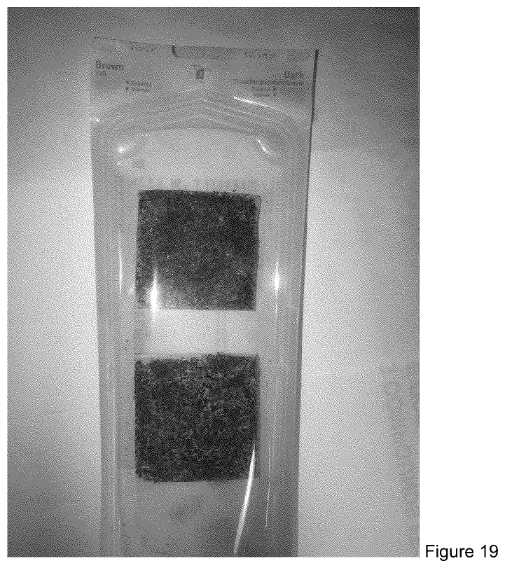

A product of the invention may be provided as a kit, for example, in a container, as shown in FIG. 19. FIG. 19 depicts a product comprising a collagen substrate having a layer of MEF provided thereon. The collagen/MEF combination is provided in a container.

For the sake of clarity, it is noted that FIG. 18 depicts two products which have been

### Example Embodiments (EEs)

1. A product comprising a biodegradable substrate, a dressing, and optionally a therapeutically acceptable bivalve extract and a therapeutically acceptable echinoderm extract.
2. The product of EE 1, wherein the product is suitable for administration to a subject.
3. The product of EE 1 or 2, wherein the product is suitable for topical administration to a subject.
4. The product of EE 3, wherein the dressing comprises an adhesive.
5. The product of EE 2 or EE 3, comprising
a. a first layer comprising the substrate; and
b. a second layer comprising the dressing
   wherein either
   i) the first layer further comprises the bivalve extract and the echinoderm extract; or
   ii) the product comprises a third layer comprising the bivalve extract and the echinoderm extract.
6. The product of EE 5, wherein the product comprises the three layers and the second layer is between the first layer and the third layer.
7. The product of EE 5, wherein the product comprises the three layers and the third layer is between the second layer and the first layer.
8. The product of any of EEs 3-7, wherein the dressing is made from a material having a high moisture vapor permeability greater than 2,500 gms/m²/24 hours when measured at 37° C. and 100% to 10% relative humidity and further wherein the dressing contains perforations.
9. The product of any of the preceding product EEs, wherein the biodegradable substrate comprises one or more ECM proteins.
10. The product of the immediately preceding EE, wherein the one or more ECM proteins are selected from a collagen, a fibronectin, a fibrin, a fibrinogen, a laminin, an elastin, a gelatin, and a vitronectin.
11. The product of the immediately preceding EE, wherein the biodegradable substrate comprises collagen Type I and/or collagen Type III.
12. The product of any of the preceding product EEs, wherein the biodegradable substrate is provided a gel or a dried substrate.
13. The product of any of the preceding product EEs, wherein the bivalve extract and the echinoderm extract are formulated for topical administration, optionally provided in a form selected from dried particles, a cream, emulsion gel, plasma, lotion, ointment, powder, capsule, liquid, spray, foam, solution, facial mask, and skin mask.
14. The product of any of the preceding product EEs, wherein or both of the bivalve extract and the echinoderm extract are a polar extract or a non-polar extract.
15. The product of any of the preceding product EEs, wherein the bivalve is from an order selected from the group consisting of Actinodontida, Adapedonta, Afghanodesmatida, Arcida, Cardiida, Carditida, Colpomyida, Cyrtodontida, Fordillida, Hippuritida, Limida, Lucinida, Megalodontida, Modiomorphida, Myalinida, Myida, Mytilida, Nuculanida, Nuculida, Ostreida, Pectinida, Solemyida, Trigoniida, Tuarangiida, Unionida, and Venerida.
16. The product of the immediately preceding EE, wherein the bivalve is from the order Pectinida.
17. The product of the immediately preceding EE, wherein the echinoderm is selected from a starfish, a sea star, a feather star, a brittle star, and an urchin.
18. The product of any of the preceding product EEs, wherein the echinoderm is from a class selected from Asteroidea, Ophiuroidea, Echinoidea, Crinoidea, and Holothuroidea.
19. The product of any of the preceding product EEs, wherein the echinoderm is from a class other than Holothuroidea.
20. The product of the immediately preceding EE, wherein the echinoderm is from the class Echinoidea.
21. The product of the immediately preceding EE, wherein the bivalve is selected from a clam, an oyster, a cockle, a mussel, and a scallop.
22. The product of any of the preceding product EEs, wherein the bivalve is a first bivalve and wherein the product further comprises an extract from a second bivalve that is not the first bivalve.
23. The product of the immediately preceding EE, wherein the first bivalve and the second bivalve are from different orders.
24. The product of the immediately preceding EE, wherein the two orders are Pectinida and Venerida.
25. The product of any of the preceding product EEs, wherein the bivalve is selected from a clam, an oyster, a cockle, a mussel, and a scallop.
26. The product of any of the preceding product EEs, wherein the echinoderm is selected from a starfish, a sea star, a feather star, a brittle star, and an urchin.
27. The product of any of the preceding product EEs, wherein the combination of bivalve and echinoderm is one of the following: the bivalve is an oyster and the echinoderm is a brittle star; the bivalve is a cockle and the echinoderm is a sea star; the bivalve is a cockle and the echinoderm is a feather star; the bivalve is a mussel and the echinoderm is a sea star; the bivalve is a scallop and the echinoderm is a starfish; the bivalve is a clam and the echinoderm is a starfish; the bivalve is a clam and the echinoderm is a brittle star; the bivalve is an oyster and the echinoderm is a sea star; the bivalve is a cockle and the echinoderm is a feather star; the bivalve is a mussel and the echinoderm is an urchin; the bivalve is a cockle and the echinoderm is a brittle star; the bivalve is a cockle and the echinoderm is an urchin; the bivalve is a mussel and the echinoderm is a brittle star; the bivalve is a scallop and the echinoderm is a brittle star; the bivalve is an oyster and the echinoderm is a starfish; the bivalve is an oyster and the echinoderm is a feather star; the bivalve is an oyster and the echinoderm is an urchin; the bivalve is a cockle and the echinoderm is an urchin; the bivalve is a cockle and the echinoderm is a brittle star; the bivalve is a scallop and the echinoderm is a sea star; the bivalve is a scallop and the echinoderm is an urchin; the bivalve is a clam and the echinoderm is an urchin; the bivalve is a clam and the echinoderm is a feather star; the bivalve is a cockle and the echinoderm is a starfish; the bivalve is a cockle and the echinoderm is a sea star; the bivalve is a mussel and the echinoderm is a feather star; the bivalve is a clam and the echinoderm is a sea star; the bivalve is a cockle and the echinoderm is a starfish; the bivalve is a scallop and the echinoderm is a feather star; or the bivalve is a mussel and the echinoderm is a starfish.
28. The product of the immediately preceding EE, further comprising an extract from a second bivalve.
29. The product of EE 28, wherein the second bivalve optionally wherein the second bivalve is an oyster.
30. The product of EE 28, wherein the second bivalve is a scallop.
31. The product of EE 28, wherein the second bivalve is a mussel.
32. The product of EE 28, wherein the second bivalve is a cockle.
33. The product of EE 28, wherein the second bivalve is clam.
34. The product of EE 1, wherein the echinoderm is from the class Echinoidea.
35. The product of the immediately preceding EE, wherein the bivalve is a first bivalve and wherein the product further comprises an extract from a second bivalve.
36. The product of EE 34 or 35, wherein the product further comprises collagen a a biodegradable substrate comprising collagen.
37. The product of the immediately preceding EE, wherein the collagen substrate comprises a plurality of channels.
38. The product of EE 36 or 37, wherein the product further comprises a dressing comprising an adhesive, optionally wherein the dressing comprises channels.
39. The product of any of EEs 34-38, wherein the echinoderm is a heart urchin, a sea urchin, or a sand dollar.
40. The product of any of EEs 34-39, wherein the bivalve is a clam, an oyster, a cockle, a mussel, a coquina, or a scallop.
41. The product of any of EEs 35-40, wherein the second bivalve is a clam, an oyster, a cockle, a mussel, a coquina, or a scallop.
42. The product of the immediately preceding EE, wherein the second bivalve is a clam.
43. A method comprising topically applying a product of any of the preceding product EEs to a subject in need thereof.
44. The method of EE 43, wherein the subject comprises a wound and the product is applied to the wound.
45. The method of EE 44, wherein the wound is a recalcitrant wound, optionally wherein the recalcitrant wound has failed to be treated successfully with standard of care for treating the wound.
46. The method of EE 44 wherein the wound is chronic skin wound or chronic ulcer.
50. A product comprising a therapeutically acceptable bivalve extract and an echinoderm extract.
51. The product of EE 50, further comprising a biodegradable substrate.
52. The product of EE 50 or EE 51, further comprising a dressing.
53. The product of EE 52, wherein the dressing comprises an adhesive.
54. The product of EE 51 or EE 52, comprising
a. a first layer comprising the substrate; and
b. a second layer comprising the dressing
wherein either
i) the first layer further comprises the bivalve extract and the echinoderm extract; or
ii) the product comprises a third layer comprising the bivalve extract and the echinoderm extract.
55. The product of EE 54, wherein the product comprises the three layers and the second layer is between the first layer and the third layer.
56. The product of EE 54, wherein the product comprises the three layers and the third layer is between the second layer and the first layer.
57. The product of any of EEs 52-56, wherein the dressing is made from a material having a high moisture vapor permeability greater than 2,500 gms/m²/24 hours when measured at 37° C. and 100% to 10% relative humidity and further wherein the dressing contains perforations.
58. The product of any of EEs 50-57, wherein the biodegradable substrate comprises one or more EMC proteins.
59. The product of the immediately preceding EE, wherein the one or more EMC proteins are selected from a collagen, a fibronectin, a fibrin, a fibrinogen, a laminin, an elastin, a gelatin, and a vitronectin.
60. The product of the immediately preceding EE, wherein the biodegradable substrate comprises collagen Type I and/or collagen Type III.
61. The product of any of EEs 50-60, wherein the biodegradable substrate is provided a gel or a dried substrate.
62. The product of any of EEs 50-61, wherein the bivalve extract and the echinoderm extract are formulated for topical administration, optionally provided in a form selected from dried particles, a cream, emulsion gel, plasma, lotion, ointment, powder, capsule, liquid, spray, foam, solution, facial mask, and skin mask.
63. The product of any of EEs 50-62, wherein or both of the bivalve extract and the echinoderm extract are a polar extract or a non-polar extract.
64. The product of any of EEs 60-64, wherein the bivalve is from an order selected from the group consisting of Actinodontida, Adapedonta, Afghanodesmatida, Arcida, Cardiida, Carditida, Colpomyida, Cyrtodontida, Fordillida, Hippuritida, Limida, Lucinida, Megalodontida, Modiomorphida, Myalinida, Myida, Mytilida, Nuculanida, Nuculida, Ostreida, Pectinida, Solemyida, Trigoniida, Tuarangiida, Unionida, and Venerida.
65. The product of the immediately preceding EE, wherein the bivalve is from the order Pectinida.
66. The product of the immediately preceding EE, wherein the echinoderm is selected from a starfish, a sea star, a feather star, a brittle star, and an urchin.
67. The product of any of EEs 50-66, wherein the echinoderm is from a class selected from Asteroidea, Ophiuroidea, Echinoidea, Crinoidea, and Holothuroidea.
68. The product of any of EEs 50-67, wherein the echinoderm is from a class other than Holothuroidea.
69. The product of the immediately preceding EE, wherein the echinoderm is from the class Echinoidea.
70. The product of the immediately preceding EE, wherein the bivalve is selected from a clam, an oyster, a cockle, a mussel, and a scallop.
71. The product of any of EEs 50-70, wherein the bivalve is a first bivalve and wherein the product further comprises an extract from a second bivalve that is not the first bivalve.
72. The product of the immediately preceding EE, wherein the first bivalve and the second bivalve are from different orders.
73. The product of the immediately preceding EE, wherein the two orders are Pectinida and Venerida.
74. The product of any of EEs 50-73, wherein the bivalve is selected from a clam, an oyster, a cockle, a mussel, and a scallop.
75. The product of any of EEs 50-74, wherein the echinoderm is selected from a starfish, a sea star, a feather star, a brittle star, and an urchin.
76. The product of any of EEs 50-75, wherein the combination of bivalve and echinoderm is one of the following: the bivalve is an oyster and the echinoderm is a brittle star; the bivalve is a cockle and the echinoderm is a sea star; the bivalve is a cockle and the echinoderm is a feather star; the bivalve is a mussel and the echinoderm is a sea star; the bivalve is a scallop and the echinoderm is a starfish; the bivalve is a clam and the echinoderm is a starfish; the bivalve is a clam and the echinoderm is a brittle star; the bivalve is an oyster and the echinoderm is a sea star; the bivalve is a cockle and the echinoderm is a feather star; the bivalve is a mussel and the echinoderm is an urchin; the bivalve is a cockle and the echinoderm is a brittle star; the bivalve is a cockle and the echinoderm is an urchin; the bivalve is a mussel and the echinoderm is a brittle star; the bivalve is a scallop and the echinoderm is a brittle star; the bivalve is an oyster and the echinoderm is a starfish; the bivalve is an oyster and the echinoderm is a feather star; the bivalve is an oyster and the echinoderm is an urchin; the bivalve is a cockle and the echinoderm is an urchin; the bivalve is a cockle and the echinoderm is a brittle star; the bivalve is a scallop and the echinoderm is a sea star; the bivalve is a scallop and the echinoderm is an urchin; the bivalve is a clam and the echinoderm is an urchin; the bivalve is a clam and the echinoderm is a feather star; the bivalve is a cockle and the echinoderm is a starfish; the bivalve is a cockle and the echinoderm is a sea star; the bivalve is a mussel and the echinoderm is a feather star; the bivalve is a clam and the echinoderm is a sea star; the bivalve is a cockle and the echinoderm is a starfish; the bivalve is a scallop and the echinoderm is a feather star; or the bivalve is a mussel and the echinoderm is a starfish.
77. The product of the immediately preceding EE, further comprising an extract from a second bivalve.
78. The product of EE 77, wherein the second bivalve optionally wherein the second bivalve is an oyster.
79. The product of EE 77, wherein the second bivalve is a scallop.
80. The product of EE 77, wherein the second bivalve is a mussel.
81. The product of EE 77, wherein the second bivalve is a cockle.
82. The product of EE 77, wherein the second bivalve is clam.
83. The product of EE 50, wherein the echinoderm is from the class Echinoidea.
84. The product of the immediately preceding EE, wherein the bivalve is a first bivalve and wherein the product further comprises an extract from a second bivalve.
85. The product of EE 83 or 84, wherein the product further comprises collagen or a biodegradable substrate comprising collagen.
86. The product of the immediately preceding EE, wherein the collagen substrate comprises a plurality of channels.
87. The product of EE 85 or 86, wherein the product further comprises a dressing comprising an adhesive, optionally wherein the dressing comprises channels.
88. The product of any of EEs 83-87, wherein the echinoderm is a heart urchin, a sea urchin, or a sand dollar.
89. The product of any of EEs 83-88, wherein the bivalve is a clam, an oyster, a cockle, a mussel, a coquina, or a scallop.
90. The product of any of EEs 84-89, wherein the second bivalve is a clam, an oyster, a cockle, a mussel, a coquina, or a scallop.
91. The product of the immediately preceding EE, wherein the second bivalve is a clam.
92. A method comprising topically applying a product of any of EEs 50-90 to a subject in need thereof.
93. The method of EE 92, wherein the subject comprises a wound and the product is applied to the wound.
94. The method of EE 93, wherein the wound is a recalcitrant wound, optionally wherein the recalcitrant wound has failed to be treated successfully with standard of care for treating the wound.
95. The method of EE 93 wherein the wound is chronic skin wound or chronic ulcer.
100. A product comprising a) a biodegradable substrate; b) one or both of a therapeutically acceptable bivalve extract and a therapeutically acceptable echinoderm extract; and c) optionally a dressing.
101. The product of EE 100, wherein the product is suitable for administration to a subject.
102. The product of EE 100 or 101, wherein the product is suitable for topical administration to a subject.
103. The product of EE 102, wherein the dressing comprises an adhesive.
104. The product of EE 101 or EE 102, comprising
a. a first layer comprising the substrate; and
b. a second layer comprising the dressing
wherein either
i) the first layer further comprises the bivalve extract and/or the echinoderm extract; or
ii) the product comprises a third layer comprising the bivalve extract and/or the echinoderm extract.
105. The product of EE 104, wherein the product comprises the three layers and the second layer is between the first layer and the third layer.
106. The product of EE 104, wherein the product comprises the three layers and the third layer is between the second layer and the first layer.
107. The product of any of EEs 102-106, wherein the dressing is made from a material having a high moisture vapor permeability greater than 2,500 gms/m²/24 hours when measured at 37° C. and 100% to 10% relative humidity and further wherein the dressing contains perforations.
108. The product of any of EEs 100-107, wherein the biodegradable substrate comprises one or more ECM proteins.
109. The product of the immediately preceding EE, wherein the one or more ECM proteins are selected from a collagen, a fibronectin, a fibrin, a fibrinogen, a laminin, an elastin, a gelatin, and a vitronectin.
110. The product of the immediately preceding EE, wherein the biodegradable substrate comprises collagen Type I and/or collagen Type III.
111. The product of any of EEs 100-110, wherein the biodegradable substrate is provided a gel or a dried substrate.
112. The product of any of EEs 100-111, wherein the bivalve extract and/or the echinoderm extract are formulated for topical administration, optionally provided in a form selected from dried particles, a cream, emulsion gel, plasma, lotion, ointment, powder, capsule, liquid, spray, foam, solution, facial mask, and skin mask.
113. The product of any of EEs 100-112, wherein or both of the bivalve extract and the echinoderm extract are a polar extract or a non-polar extract.
114. The product of any of EEs 100-113, wherein the bivalve is from an order selected from the group consisting of Actinodontida, Adapedonta, Afghanodesmatida, Arcida, Cardiida, Carditida, Colpomyida, Cyrtodontida, Fordillida, Hippuritida, Limida, Lucinida, Megalodontida, Modiomorphida, Myalinida, Myida, Mytilida, Nuculanida, Nuculida, Ostreida, Pectinida, Solemyida, Trigoniida, Tuarangiida, Unionida, and Venerida.
115. The product of the immediately preceding EE, wherein the bivalve is from the order Pectinida.
116. The product of the immediately preceding EE, wherein the echinoderm is selected from a starfish, a sea star, a feather star, a brittle star, and an urchin.
117. The product of any of EEs 100-116, wherein the echinoderm is from a class selected from Asteroidea, Ophiuroidea, Echinoidea, Crinoidea, and Holothuroidea.
118. The product of any of EEs 100-117, wherein the echinoderm is from a class other than Holothuroidea.
119. The product of the immediately preceding EE, wherein the echinoderm is from the class Echinoidea.
120. The product of the immediately preceding EE, wherein the bivalve is selected from a clam, an oyster, a cockle, a mussel, and a scallop.
121. The product of any of EEs 100-120, wherein the bivalve is a first bivalve and wherein the product further comprises an extract from a second bivalve that is not the first bivalve.
122. The product of the immediately preceding EE, wherein the first bivalve and the second bivalve are from different orders.
123. The product of the immediately preceding EE, wherein the two orders are Pectinida and Venerida.
124. The product of any of EEs 100-123, wherein the bivalve is selected from a clam, an oyster, a cockle, a mussel, and a scallop.
125. The product of any of EEs 100-124, wherein the echinoderm is selected from a starfish, a sea star, a feather star, a brittle star, and an urchin.
126. The product of any of EEs 100-125, wherein the combination of bivalve and echinoderm is one of the following: the bivalve is an oyster and the echinoderm is a brittle star; the bivalve is a cockle and the echinoderm is a sea star; the bivalve is a cockle and the echinoderm is a feather star; the bivalve is a mussel and the echinoderm is a sea star; the bivalve is a scallop and the echinoderm is a starfish; the bivalve is a clam and the echinoderm is a starfish; the bivalve is a clam and the echinoderm is a brittle star; the bivalve is an oyster and the echinoderm is a sea star; the bivalve is a cockle and the echinoderm is a feather star; the bivalve is a mussel and the echinoderm is an urchin; the bivalve is a cockle and the echinoderm is a brittle star; the bivalve is a cockle and the echinoderm is an urchin; the bivalve is a mussel and the echinoderm is a brittle star; the bivalve is a scallop and the echinoderm is a brittle star; the bivalve is an oyster and the echinoderm is a starfish; the bivalve is an oyster and the echinoderm is a feather star; the bivalve is an oyster and the echinoderm is an urchin; the bivalve is a cockle and the echinoderm is an urchin; the bivalve is a cockle and the echinoderm is a brittle star; the bivalve is a scallop and the echinoderm is a sea star; the bivalve is a scallop and the echinoderm is an urchin; the bivalve is a clam and the echinoderm is an urchin; the bivalve is a clam and the echinoderm is a feather star; the bivalve is a cockle and the echinoderm is a starfish; the bivalve is a cockle and the echinoderm is a sea star; the bivalve is a mussel and the echinoderm is a feather star; the bivalve is a clam and the echinoderm is a sea star; the bivalve is a cockle and the echinoderm is a starfish; the bivalve is a scallop and the echinoderm is a feather star; or the bivalve is a mussel and the echinoderm is a starfish.
127. The product of the immediately preceding EE, further comprising an extract from a second bivalve.
128. The product of EE 127, wherein the second bivalve optionally wherein the second bivalve is an oyster.
129. The product of EE 127, wherein the second bivalve is a scallop.
130. The product of EE 127, wherein the second bivalve is a mussel.
131. The product of EE 127, wherein the second bivalve is a cockle.
132. The product of EE 127, wherein the second bivalve is clam.
133. The product of EE 100, wherein the echinoderm is from the class Echinoidea.
134. The product of the immediately preceding EE, wherein the bivalve is a first bivalve and wherein the product further comprises an extract from a second bivalve.
135. The product of EE 133 or 134, wherein the product further comprises collagen a a biodegradable substrate comprising collagen.
136. The product of the immediately preceding EE, wherein the collagen substrate comprises a plurality of channels.
137. The product of EE 135 or 136, wherein the product further comprises a dressing comprising an adhesive, optionally wherein the dressing comprises channels.
138. The product of any of EEs 133-137, wherein the echinoderm is a heart urchin, a sea urchin, or a sand dollar.
139. The product of any of EEs 133-138, wherein the bivalve is a clam, an oyster, a cockle, a mussel, a coquina, or a scallop.
140. The product of any of EEs 134-139, wherein the second bivalve is a clam, an oyster, a cockle, a mussel, a coquina, or a scallop.
141. The product of the immediately preceding EE, wherein the second bivalve is a clam.
142. A method comprising topically applying a product of any of EEs 100-141 to a subject in need thereof.
143. The method of EE 142, wherein the subject comprises a wound and the product is applied to the wound.
144. The method of EE 143, wherein the wound is a recalcitrant wound, optionally wherein the recalcitrant wound has failed to be treated successfully with standard of care for treating the wound.
145. The method of EE 143 wherein the wound is chronic skin wound or chronic ulcer.
150. A product comprising a) a biodegradable substrate; b) a dressing; and c) optionally one or both of a therapeutically acceptable bivalve extract and a therapeutically acceptable echinoderm extract.
151. The product of EE 150, wherein the product is suitable for administration to a subject.
152. The product of EE 150 or 151, wherein the product is suitable for topical administration to a subject.
153. The product of EE 152, wherein the dressing comprises an adhesive.
154. The product of EE 151 or EE 152, comprising
a. a first layer comprising the substrate; and
b. a second layer comprising the dressing
wherein either
i) the first layer further comprises the bivalve extract and/or the echinoderm extract; or
ii) the product comprises a third layer comprising the bivalve extract and/or the echinoderm extract.
155. The product of EE 154, wherein the product comprises the three layers and the second layer is between the first layer and the third layer.
156. The product of EE 154, wherein the product comprises the three layers and the third layer is between the second layer and the first layer.
157. The product of any of EEs 152-156, wherein the dressing is made from a material having a high moisture vapor permeability greater than 2,500 gms/m²/24 hours when measured at 37° C. and 100% to 10% relative humidity and further wherein the dressing contains perforations.
158. The product of any of EEs 150-157, wherein the biodegradable substrate comprises one or more ECM proteins.
159. The product of the immediately preceding EE, wherein the one or more ECM proteins are selected from a collagen, a fibronectin, a fibrin, a fibrinogen, a laminin, an elastin, a gelatin, and a vitronectin.
160. The product of the immediately preceding EE, wherein the biodegradable substrate comprises collagen Type I and/or collagen Type III.
161. The product of any of EEs 150-160, wherein the biodegradable substrate is provided a gel or a dried substrate.
162. The product of any of EEs 150-161, wherein the bivalve extract and the echinoderm extract are formulated for topical administration, optionally provided in a form selected from dried particles, a cream, emulsion gel, plasma, lotion, ointment, powder, capsule, liquid, spray, foam, solution, facial mask, and skin mask.
163. The product of any of EEs 150-162, wherein or both of the bivalve extract and the echinoderm extract are a polar extract or a non-polar extract.
164. The product of any of EEs 150-163, wherein the bivalve is from an order selected from the group consisting of Actinodontida, Adapedonta, Afghanodesmatida, Arcida, Cardiida, Carditida, Colpomyida, Cyrtodontida, Fordillida, Hippuritida, Limida, Lucinida, Megalodontida, Modiomorphida, Myalinida, Myida, Mytilida, Nuculanida, Nuculida, Ostreida, Pectinida, Solemyida, Trigoniida, Tuarangiida, Unionida, and Venerida.
165. The product of the immediately preceding EE, wherein the bivalve is from the order Pectinida.
166. The product of the immediately preceding EE, wherein the echinoderm is selected from a starfish, a sea star, a feather star, a brittle star, and an urchin.
167. The product of any of EEs 150-166, wherein the echinoderm is from a class selected from Asteroidea, Ophiuroidea, Echinoidea, Crinoidea, and Holothuroidea.
168. The product of any of EEs 150-167, wherein the echinoderm is from a class other than Holothuroidea.
169. The product of the immediately preceding EE, wherein the echinoderm is from the class Echinoidea.
170. The product of the immediately preceding EE, wherein the bivalve is selected from a clam, an oyster, a cockle, a mussel, and a scallop.
171. The product of any of EEs 150-170, wherein the bivalve is a first bivalve and wherein the product further comprises an extract from a second bivalve that is not the first bivalve.
172. The product of the immediately preceding EE, wherein the first bivalve and the second bivalve are from different orders.
173. The product of the immediately preceding EE, wherein the two orders are Pectinida and Venerida.
174. The product of any of EEs 150-173, wherein the bivalve is selected from a clam, an oyster, a cockle, a mussel, and a scallop.
175. The product of any of EEs 150-174, wherein the echinoderm is selected from a starfish, a sea star, a feather star, a brittle star, and an urchin.
176. The product of any of EEs 150-175, wherein the combination of bivalve and echinoderm is one of the following: the bivalve is an oyster and the echinoderm is a brittle star; the bivalve is a cockle and the echinoderm is a sea star; the bivalve is a cockle and the echinoderm is a feather star; the bivalve is a mussel and the echinoderm is a sea star; the bivalve is a scallop and the echinoderm is a starfish; the bivalve is a clam and the echinoderm is a starfish; the bivalve is a clam and the echinoderm is a brittle star; the bivalve is an oyster and the echinoderm is a sea star; the bivalve is a cockle and the echinoderm is a feather star; the bivalve is a mussel and the echinoderm is an urchin; the bivalve is a cockle and the echinoderm is a brittle star; the bivalve is a cockle and the echinoderm is an urchin; the bivalve is a mussel and the echinoderm is a brittle star; the bivalve is a scallop and the echinoderm is a brittle star; the bivalve is an oyster and the echinoderm is a starfish; the bivalve is an oyster and the echinoderm is a feather star; the bivalve is an oyster and the echinoderm is an urchin; the bivalve is a cockle and the echinoderm is an urchin; the bivalve is a cockle and the echinoderm is a brittle star; the bivalve is a scallop and the echinoderm is a sea star; the bivalve is a scallop and the echinoderm is an urchin; the bivalve is a clam and the echinoderm is an urchin; the bivalve is a clam and the echinoderm is a feather star; the bivalve is a cockle and the echinoderm is a starfish; the bivalve is a cockle and the echinoderm is a sea star; the bivalve is a mussel and the echinoderm is a feather star; the bivalve is a clam and the echinoderm is a sea star; the bivalve is a cockle and the echinoderm is a starfish; the bivalve is a scallop and the echinoderm is a feather star; or the bivalve is a mussel and the echinoderm is a starfish.
177. The product of the immediately preceding EE, further comprising an extract from a second bivalve.
178. The product of EE 177, wherein the second bivalve optionally wherein the second bivalve is an oyster.
179. The product of EE 177, wherein the second bivalve is a scallop.
180. The product of EE 177, wherein the second bivalve is a mussel.
181. The product of EE 177, wherein the second bivalve is a cockle.
182. The product of EE 177, wherein the second bivalve is clam.
183. The product of EE 150, wherein the echinoderm is from the class Echinoidea.
184. The product of the immediately preceding EE, wherein the bivalve is a first bivalve and wherein the product further comprises an extract from a second bivalve.
185. The product of EE 183 or 184, wherein the product further comprises collagen a a biodegradable substrate comprising collagen.
186. The product of the immediately preceding EE, wherein the collagen substrate comprises a plurality of channels.
187. The product of EE 185 or 186, wherein the product further comprises a dressing comprising an adhesive, optionally wherein the dressing comprises channels.
188. The product of any of EEs 183-187, wherein the echinoderm is a heart urchin, a sea urchin, or a sand dollar.
189. The product of any of EEs 183-188, wherein the bivalve is a clam, an oyster, a cockle, a mussel, a coquina, or a scallop.
190. The product of any of EEs 184-189, wherein the second bivalve is a clam, an oyster, a cockle, a mussel, a coquina, or a scallop.
191. The product of the immediately preceding EE, wherein the second bivalve is a clam.
192. A method comprising topically applying a product of any of EEs 150-191 to a subject in need thereof.
193. The method of EE 192, wherein the subject comprises a wound and the product is applied to the wound.
194. The method of EE 193, wherein the wound is a recalcitrant wound, optionally wherein the recalcitrant wound has failed to be treated successfully with standard of care for treating the wound.
195. The method of EE 193 wherein the wound is chronic skin wound or chronic ulcer.
200. A product comprising a) a biodegradable substrate; b) a dressing; and c) optionally a dermatologically acceptable extract.
201. The product of EE 200, wherein the product is suitable for administration to a subject.
202. The product of EE 200 or 201, wherein the product is suitable for topical administration to a subject.
203. The product of EE 202, wherein the dressing comprises an adhesive.
204. The product of EE 201 or EE 202, comprising
a. a first layer comprising the substrate; and
b. a second layer comprising the dressing
wherein either
i) the first layer further comprises the extract; or
ii) the product comprises a third layer comprising the extract.
205. The product of EE 204, wherein the product comprises the three layers and the second layer is between the first layer and the third layer.
206. The product of EE 204, wherein the product comprises the three layers and the third layer is between the second layer and the first layer.
207. The product of any of EEs 202-206, wherein the dressing is made from a material having a high moisture vapor permeability greater than 2,500 gms/m²/24 hours when measured at 37° C. and 100% to 10% relative humidity and further wherein the dressing contains perforations.
208. The product of any of EEs 200-207, wherein the biodegradable substrate comprises one or more ECM proteins.
209. The product of the immediately preceding EE, wherein the one or more ECM proteins are selected from a collagen, a fibronectin, a fibrin, a fibrinogen, a laminin, an elastin, a gelatin, and a vitronectin.
210. The product of the immediately preceding EE, wherein the biodegradable substrate comprises collagen Type I and/or collagen Type III.
211. The product of any of EEs 200-210, wherein the biodegradable substrate is provided a gel or a dried substrate.
212. The product of any of EEs 200-211, wherein extract is formulated for topical administration, optionally provided in a form selected from dried particles, a cream, emulsion gel, plasma, lotion, ointment, powder, capsule, liquid, spray, foam, solution, facial mask, and skin mask.
213. The product of any of EEs 200-212, wherein the extract is a polar extract or a non-polar extract.
214. The product of any of EEs 200-213, wherein the extract comprises an extract of an organism taught in U.S. patent Ser. No. 10/772,825 issued Sep. 15, 2020 to Eric Lewis.
215. The product of any of EEs 200-213, wherein the extract is an extract or a topical composition taught in U.S. patent Ser. No. 10/772,825 issued Sep. 15, 2020 to Eric Lewis.
216. The product of any of EEs 200-213, wherein the extract is from a marine animal or marine plant.
217. The product of any of EEs 200-213, wherein the extract comprises an extract from a source selected from Aloe arborescens leaf; Aloe aristata leaf; Aloe ferox leaf; Bulbine frutescens leaf; Bulbine frutescens root; Bulbine natalensis leaf; Bulbine natalensis root; Eucomis autumnalis leaf; Eucomis autumnalis bulb/root; Haworthia limifolia leaf; Hypericum aethiopicum leaf; Merwilla plumbea leaf; Merwilla plumbea bulb; Merwilla plumbea root; Tetradenia riparia leaf; Tetradenia riparia stem; Zantedeschia aethiopica leaf; Zantedeschia aethiopica stem, or the genus of any of the aforementioned organisms.
218. The product of any of EEs 200-213, wherein the extract comprises an extract from a source selected from Aloe megalacantha Baker (Leaf latex); Phyllanthus muellerianus (water extract of aerial part and pure compound geraniin); The biofunctionalized silver nanoparticle was produced from cloves extract; Euphorbia characias subsp. Wulfenii; Lafoensia pacari A. St.-Hil; Alkanna strigose; Vitis labrusca; Coccinia grandis; Jacaranda decurrens; Phlomis russeliana; Plumeria obtusa; Boerhavia diffusa; Ephedra ciliata; Moringa oliefera; Moringa oleifera leaves; Curatella americana Linn.; Nigella sativa oil; Dodonaea viscosa; Roylea elegans; Cupressus macrocarpa; Zehneria scabra; Bersama abyssinica; Semecarpus anacardium L., Argemone mexicana L., Cocculus hirsutus L., Woodfordia fruticose K; Elaeis guineensis Jacq (Leaves); Vernonia auriculifera Hiern; Brucea antidysentrica Rhamnus prinoides Dodonaea angustifolia; Jatropha Neopauciflora Pax Latex; Sanguisorba officinalis Roots; Platycodon grandifloras; Pistacia vera; Moringa oleifera; Calendula officinalis L.; Curcuma longa; Globularia arabica; Premna integrifolia; Zizyphus mauritiana; and Parkia clappertoniana Keay, or the genus of any of the aforementioned organisms.
219. The product of any of EEs 200-213, wherein the extract comprises an extract from a family selected from Xanthorrhoeaceae; Euphorbiaceae; Myrtaceae; Euphorbiaceae; Lythraceae; Boraginaceae; Vitaceae; Cucurbitaceae; Bignoniaceae; Lamiaceae; Apocynaceae; Nyctaginaceae; Ephedraceae; Moringaceae; Moringaceae; Dilleniaceae; Ranunculaceae; Sapindaceae; Lamiaceae; Cupressaceae; Cucurbitaceae; Francoaceae; Anacardiaceae Papaveraceaei Menispermaceae Lythraceae; Arecaceae; Asteraceae; Simaroubaceae; Euphorbiaceae; Rosoideae; Campanulaceae; Anacardiaceae; Moringaceae; Asteraceae; Zingiberaceae; Plantaginaceae; Lamiaceae; Rhamnaceae; and Fabaceae.
220. The product of any of EEs 200-219, wherein the product comprises collagen or the biodegradable substrate comprises collagen.
221. The product of the immediately preceding EE, wherein the collagen substrate comprises a plurality of channels.
222. The product of EE 220 or 221, wherein the product further comprises a dressing comprising an adhesive, optionally wherein the dressing comprises channels.
223. A method comprising topically applying a product of any of EEs 200-222 to a subject in need thereof.
224. The method of EE 223, wherein the subject comprises a wound and the product is applied to the wound.
225. The method of EE 224, wherein the wound is a recalcitrant wound, optionally wherein the recalcitrant wound has failed to be treated successfully with standard of care for treating the wound.
226. The method of EE 224 wherein the wound is chronic skin wound or chronic ulcer.
250. A product comprising a) a dermatologically acceptable extract; and b) one or both of a biodegradable substrate and a dressing.
251. The product of EE 250, wherein the product is suitable for administration to a subject.
252. The product of EE 250 or 251, wherein the product is suitable for topical administration to a subject.
253. The product of EE 252, wherein the dressing comprises an adhesive.
254. The product of EE 251 or EE 252, comprising
a. a first layer comprising the substrate; and
b. a second layer comprising the dressing
wherein either
i) the first layer further comprises the extract; or
ii) the product comprises a third layer comprising the extract.
255. The product of EE 254, wherein the product comprises the three layers and the second layer is between the first layer and the third layer.
256. The product of EE 254, wherein the product comprises the three layers and the third layer is between the second layer and the first layer.
257. The product of any of EEs 252-256, wherein the dressing is made from a material having a high moisture vapor permeability greater than 2,500 gms/m²/24 hours when measured at 37° C. and 100% to 10% relative humidity and further wherein the dressing contains perforations.
258. The product of any of EEs 250-257, wherein the biodegradable substrate comprises one or more ECM proteins.
259. The product of the immediately preceding EE, wherein the one or more ECM proteins are selected from a collagen, a fibronectin, a fibrin, a fibrinogen, a laminin, an elastin, a gelatin, and a vitronectin.
260. The product of the immediately preceding EE, wherein the biodegradable substrate comprises collagen Type I and/or collagen Type III.
261. The product of any of EEs 250-260, wherein the biodegradable substrate is provided a gel or a dried substrate.
262. The product of any of EEs 250-261, wherein extract is formulated for topical administration, optionally provided in a form selected from dried particles, a cream, emulsion gel, plasma, lotion, ointment, powder, capsule, liquid, spray, foam, solution, facial mask, and skin mask.
263. The product of any of EEs 250-262, wherein the extract is a polar extract or a non-polar extract.
264. The product of any of EEs 250-263, wherein the extract comprises an extract of an organism taught in U.S. patent Ser. No. 10/772,825 issued Sep. 15, 2020 to Eric Lewis.
265. The product of any of EEs 250-263, wherein the extract is an extract or a topical composition taught in U.S. patent Ser. No. 10/772,825 issued Sep. 15, 2020 to Eric Lewis.
266. The product of any of EEs 250-263, wherein the extract is from a marine animal or marine plant.
267. The product of any of EEs 250-263, wherein the extract comprises an extract from a source selected from Aloe arborescens leaf; Aloe aristata leaf; Aloe ferox leaf; Bulbine frutescens leaf; Bulbine frutescens root; Bulbine natalensis leaf; Bulbine natalensis root; Eucomis autumnalis leaf; Eucomis autumnalis bulb/root; Haworthia limifolia leaf; Hypericum aethiopicum leaf; Merwilla plumbea leaf; Merwilla plumbea bulb; Merwilla plumbea root; Tetradenia riparia leaf; Tetradenia riparia stem; Zantedeschia aethiopica leaf; Zantedeschia aethiopica stem, or the genus of any of the aforementioned organisms.
268. The product of any of EEs 250-263, wherein the extract comprises an extract from a source selected from Aloe megalacantha Baker (Leaf latex); Phyllanthus muellerianus (water extract of aerial part and pure compound geraniin); The biofunctionalized silver nanoparticle was produced from cloves extract; Euphorbia characias subsp. Wulfenii; Lafoensia pacari A. St.-Hil; Alkanna strigose; Vitis labrusca; Coccinia grandis; Jacaranda decurrens; Phlomis russeliana; Plumeria obtusa; Boerhavia diffusa; Ephedra ciliata; Moringa oliefera; Moringa oleifera leaves; Curatella americana Linn.; Nigella sativa oil; Dodonaea viscosa; Roylea elegans; Cupressus macrocarpa; Zehneria scabra; Bersama abyssinica; Semecarpus anacardium L., Argemone mexicana L., Cocculus hirsutus L., Woodfordia fruticose K; Elaeis guineensis Jacq (Leaves); Vernonia auriculifera Hiern; Brucea antidysentrica Rhamnus prinoides Dodonaea angustifolia; Jatropha Neopauciflora Pax Latex; Sanguisorba officinalis Roots; Platycodon grandifloras; Pistacia vera; Moringa oleifera; Calendula officinalis L.; Curcuma longa; Globularia arabica; Premna integrifolia; Zizyphus mauritiana; and Parkia clappertoniana Keay, or the genus of any of the aforementioned organisms.
269. The product of any of EEs 250-263, wherein the extract comprises an extract from a family selected from Xanthorrhoeaceae; Euphorbiaceae; Myrtaceae; Euphorbiaceae; Lythraceae; Boraginaceae; Vitaceae; Cucurbitaceae; Bignoniaceae; Lamiaceae; Apocynaceae; Nyctaginaceae; Ephedraceae; Moringaceae; Moringaceae; Dilleniaceae; Ranunculaceae; Sapindaceae; Lamiaceae; Cupressaceae; Cucurbitaceae; Francoaceae; Anacardiaceae Papaveraceaei Menispermaceae Lythraceae; Arecaceae; Asteraceae; Simaroubaceae; Euphorbiaceae; Rosoideae; Campanulaceae; Anacardiaceae; Moringaceae; Asteraceae; Zingiberaceae; Plantaginaceae; Lamiaceae; Rhamnaceae; and Fabaceae.
270. The product of any of EEs 250-269, wherein the product comprises collagen or the biodegradable substrate comprises collagen.
271. The product of the immediately preceding EE, wherein the collagen substrate comprises a plurality of channels.
272. The product of EE 270 or 271, wherein the product further comprises a dressing comprising an adhesive, optionally wherein the dressing comprises channels.
273. A method comprising topically applying a product of any of EEs 250-272 to a subject in need thereof.
274. The method of EE 273, wherein the subject comprises a wound and the product is applied to the wound.
275. The method of EE 274, wherein the wound is a recalcitrant wound, optionally wherein the recalcitrant wound has failed to be treated successfully with standard of care for treating the wound.
276. The method of EE 274 wherein the wound is chronic skin wound or chronic ulcer.
300. A product comprising a) a collagen substrate; b) one or both of a therapeutically acceptable bivalve extract and a therapeutically acceptable echinoderm extract; and c) optionally a dressing.
301. The product of EE 300, wherein the product is suitable for administration to a subject.
302. The product of EE 300 or 301, wherein the product is suitable for topical administration to a subject.
303. The product of EE 302, wherein the dressing comprises an adhesive.
304. The product of EE 301 or EE 302, comprising
a. a first layer comprising the substrate; and
b. a second layer comprising the dressing
wherein either
i) the first layer further comprises the bivalve extract and/or the echinoderm extract; or
ii) the product comprises a third layer comprising the bivalve extract and/or the echinoderm extract.
305. The product of EE 304, wherein the product comprises the three layers and the second layer is between the first layer and the third layer.
306. The product of EE 304, wherein the product comprises the three layers and the third layer is between the second layer and the first layer.
307. The product of any of EEs 302-306, wherein the dressing is made from a material having a high moisture vapor permeability greater than 2,500 gms/m²/24 hours when measured at 37° C. and 100% to 10% relative humidity and further wherein the dressing contains perforations.
308. The product of any of EEs 300-307, wherein the substrate further comprises one or more ECM proteins other than collagen.
309. The product of the immediately preceding EE, wherein the one or more other ECM proteins are selected from, a fibronectin, a fibrin, a fibrinogen, a laminin, an elastin, a gelatin, and a vitronectin.
310. The product of any of EEs 300-309, wherein the substrate comprises collagen Type I and/or collagen Type III.
311. The product of any of EEs 300-310, wherein the substrate is provided a gel or a dried substrate.
312. The product of any of EEs 300-311, wherein the substrate is substantially non flowable.
313. The product of any of EEs 300-311, wherein the substrate is elastic or viscoelastic.
314. The product of any of EEs 300-311, wherein the substrate is flexible or bendable.
315. The product of any of EEs 300-311, wherein the substrate comprises the collagen in a majority amount (w/w).
316. The product of any of EEs 300-311, wherein the substrate comprises the collagen in a majority amount (w/w) of the dry weight of the substrate.
317. The product of any of EEs 300-311, wherein the substrate comprises one or more ECM proteins other than the collagen, wherein the ECM proteins and the collagen, collectively, are present in the substrate in a majority amount (w/w).
318. The product of any of EEs 300-311, wherein the substrate comprises one or more ECM proteins other than the collagen, wherein the ECM proteins and the collagen, collectively, are present in the substrate in a majority amount (w/w) of the dry weight of the substrate.
319. The product of any of EEs 300-311, wherein the substrate has a viscosity of at least 100,000 cps, at least 200,000 cps, at least 300,000 cps, at least 400,000 cps, at least 500,000 cps, at least 1,000,000 cps, at least 10,000,000 cps, at least 20,000,000 cps, at least 30,000,000 cps, at least at least 40,000,000 cps, or at least at least 50,000,000 cps.
320. The product of any of EEs 300-311, wherein the collagen is derived from mammal tendon.
321. The product of any of EEs 300-311, wherein the collagen is derived from a source selected from mammal and marine animal, optionally wherein the mammal is bovine, equine, or porcine or wherein the marine animal is fish.
322. The product of any of EEs 300-311, wherein the collagen is type I collagen having a triple helix.
323. The product of any of EEs 300-311, wherein the substrate has a state of hydration such that the substrate can absorb at least 5× or at least 10× its weight in water.
324. The product of any of EEs 300-311, wherein the bivalve extract and/or the echinoderm extract are formulated for topical administration, optionally provided in a form selected from dried particles, a cream, emulsion gel, plasma, lotion, ointment, powder, capsule, liquid, spray, foam, solution, facial mask, and skin mask.
325. The product of any of EEs 300-324, wherein or both of the bivalve extract and the echinoderm extract are a polar extract or a non-polar extract.
326. The product of any of EEs 300-325, wherein the bivalve is from an order selected from the group consisting of Actinodontida, Adapedonta, Afghanodesmatida, Arcida, Cardiida, Carditida, Colpomyida, Cyrtodontida, Fordillida, Hippuritida, Limida, Lucinida, Megalodontida, Modiomorphida, Myalinida, Myida, Mytilida, Nuculanida, Nuculida, Ostreida, Pectinida, Solemyida, Trigoniida, Tuarangiida, Unionida, and Venerida.
327. The product of the immediately preceding EE, wherein the bivalve is from the order Pectinida.
328. The product of the immediately preceding EE, wherein the echinoderm is selected from a starfish, a sea star, a feather star, a brittle star, and an urchin.
329. The product of any of EEs 300-328, wherein the echinoderm is from a class selected from Asteroidea, Ophiuroidea, Echinoidea, Crinoidea, and Holothuroidea.
330. The product of any of EEs 300-329, wherein the echinoderm is from a class other than Holothuroidea.
331. The product of the immediately preceding EE, wherein the echinoderm is from the class Echinoidea.
332. The product of the immediately preceding EE, wherein the bivalve is selected from a clam, an oyster, a cockle, a mussel, and a scallop.
333. The product of any of EEs 300-332, wherein the bivalve is a first bivalve and wherein the product further comprises an extract from a second bivalve that is not the first bivalve.
334. The product of the immediately preceding EE, wherein the first bivalve and the second bivalve are from different orders.
335. The product of the immediately preceding EE, wherein the two orders are Pectinida and Venerida.
336. The product of any of EEs 300-335, wherein the bivalve is selected from a clam, an oyster, a cockle, a mussel, and a scallop.
337. The product of any of EEs 300-336, wherein the echinoderm is selected from a starfish, a sea star, a feather star, a brittle star, and an urchin.
338. The product of any of EEs 300-337, wherein the combination of bivalve and echinoderm is one of the following: the bivalve is an oyster and the echinoderm is a brittle star; the bivalve is a cockle and the echinoderm is a sea star; the bivalve is a cockle and the echinoderm is a feather star; the bivalve is a mussel and the echinoderm is a sea star; the bivalve is a scallop and the echinoderm is a starfish; the bivalve is a clam and the echinoderm is a starfish; the bivalve is a clam and the echinoderm is a brittle star; the bivalve is an oyster and the echinoderm is a sea star; the bivalve is a cockle and the echinoderm is a feather star; the bivalve is a mussel and the echinoderm is an urchin; the bivalve is a cockle and the echinoderm is a brittle star; the bivalve is a cockle and the echinoderm is an urchin; the bivalve is a mussel and the echinoderm is a brittle star; the bivalve is a scallop and the echinoderm is a brittle star; the bivalve is an oyster and the echinoderm is a starfish; the bivalve is an oyster and the echinoderm is a feather star; the bivalve is an oyster and the echinoderm is an urchin; the bivalve is a cockle and the echinoderm is an urchin; the bivalve is a cockle and the echinoderm is a brittle star; the bivalve is a scallop and the echinoderm is a sea star; the bivalve is a scallop and the echinoderm is an urchin; the bivalve is a clam and the echinoderm is an urchin; the bivalve is a clam and the echinoderm is a feather star; the bivalve is a cockle and the echinoderm is a starfish; the bivalve is a cockle and the echinoderm is a sea star; the bivalve is a mussel and the echinoderm is a feather star; the bivalve is a clam and the echinoderm is a sea star; the bivalve is a cockle and the echinoderm is a starfish; the bivalve is a scallop and the echinoderm is a feather star; or the bivalve is a mussel and the echinoderm is a starfish.
339. The product of the immediately preceding EE, further comprising an extract from a second bivalve.
340. The product of EE 339, wherein the second bivalve optionally wherein the second bivalve is an oyster.
341. The product of EE 339, wherein the second bivalve is a scallop.
342. The product of EE 339, wherein the second bivalve is a mussel.
343. The product of EE 339, wherein the second bivalve is a cockle.
344. The product of EE 339, wherein the second bivalve is clam.
345. The product of EE 300, wherein the echinoderm is from the class Echinoidea.
346. The product of the immediately preceding EE, wherein the bivalve is a first bivalve and wherein the product further comprises an extract from a second bivalve.
347. The product of EE 345 or 346, wherein the substrate is in the form of a sheet.
348. The product of the immediately preceding EE, wherein the substrate comprises a plurality of channels.
349. The product of EE 347 or 348, wherein the product further comprises a dressing comprising an adhesive, optionally wherein the dressing comprises channels.
350. The product of any of EEs 345-349, wherein the echinoderm is a heart urchin, a sea urchin, or a sand dollar.
351. The product of any of EEs 345-350, wherein the bivalve is a clam, an oyster, a cockle, a mussel, a coquina, or a scallop.
352. The product of any of EEs 346-351, wherein the second bivalve is a clam, an oyster, a cockle, a mussel, a coquina, or a scallop.
353. The product of the immediately preceding EE, wherein the second bivalve is a clam.
354. A method comprising topically applying a product of any of EEs 300-353 to a subject in need thereof.
355. The method of EE 354, wherein the subject comprises a wound and the product is applied to the wound.
356. The method of EE 355, wherein the wound is a recalcitrant wound, optionally wherein the recalcitrant wound has failed to be treated successfully with standard of care for treating the wound.
357. The method of EE 355 wherein the wound is chronic skin wound or chronic ulcer.
358. A product comprising a therapeutically acceptable bivalve extract and an echinoderm extract.
359. The product of EE 358, further comprising a biodegradable substrate.
360. The product of EE 358, further comprising a dressing.
361. The product of EE 360, wherein the dressing comprises an adhesive.
362. The product of EE 360, comprising
a. a first layer comprising a biodegradable substrate; and
b. a second layer comprising the dressing
   wherein either
i) the first layer further comprises the bivalve extract and the echinoderm extract; or
ii) the product comprises a third layer comprising the bivalve extract and the echinoderm extract.
363. The product of EE 362, wherein the product comprises the three layers and the second layer is between the first layer and the third layer.
364. The product of EE 362, wherein the product comprises the three layers and the third layer is between the second layer and the first layer.
365. The product of EE 360, wherein the dressing is made from a material having a high moisture vapor permeability greater than 2,500 gms/m²/24 hours when measured at 37° C. and 100% to 10% relative humidity and further wherein the dressing contains perforations.
366. The product of EE 359 wherein the biodegradable substrate comprises one or more extracellular matrix (‘ECM’) proteins.
367. The product of EE 366, wherein the one or more ECM proteins comprise any one or any combination of a collagen, a fibronectin, a fibrin, a fibrinogen, a laminin, an elastin, a gelatin, and a vitronectin.
368. The product of the EE 367, wherein the biodegradable substrate comprises collagen Type I and/or collagen Type III.
369. The product of EE 359, wherein the biodegradable substrate is provided as a plurality of particles or a sheet.
370. The product of any of EE 358, wherein the bivalve extract and the echinoderm extract are formulated for topical administration, optionally provided in a form selected from dried particles, a cream, emulsion gel, plasma, lotion, ointment, powder, capsule, liquid, spray, foam, solution, facial mask, and skin mask.
371. The product of any of EE 358, wherein or both of the bivalve extract and the echinoderm extract comprise ground tissue, a polar extract, or a non-polar extract.
372. The product of EE 358, wherein the bivalve is from an order selected from the group consisting of Actinodontida, Adapedonta, Afghanodesmatida, Arcida, Cardiida, Carditida, Colpomyida, Cyrtodontida, Fordillida, Hippuritida, Limida, Lucinida, Megalodontida, Modiomorphida, Myalinida, Myida, Mytilida, Nuculanida, Nuculida, Ostreida, Pectinida, Solemyida, Trigoniida, Tuarangiida, Unionida, and Venerida.
373. The product of EE 372, wherein the bivalve is from the order Pectinida.
374. The product of EE 373, wherein the echinoderm is selected from a starfish, a sea star, a feather star, a brittle star, and an urchin.
375. The product of EE 358, wherein the echinoderm is from a class selected from Asteroidea, Ophiuroidea, Echinoidea, Crinoidea, and Holothuroidea.
376. The product of EE 358, wherein the echinoderm is from a class other than Holothuroidea.
377. The product of EE 376, wherein the echinoderm is from the class Echinoidea.
378. The product of EE 377, wherein the bivalve is selected from a clam, an oyster, a cockle, a mussel, and a scallop.
379. The product of EE 358, wherein the bivalve is a first bivalve and wherein the product further comprises an extract from a second bivalve that is not the first bivalve.
380. The product of EE 379, wherein the first bivalve and the second bivalve are from different orders.
381. The product of EE 380, wherein the two orders are Pectinida and Venerida.
382. The product of EE 358, wherein the bivalve is selected from a clam, an oyster, a cockle, a mussel, and a scallop.
383. The product of EE 358, wherein the echinoderm is selected from a starfish, a sea star, a feather star, a brittle star, and an urchin.
384. The product of EE 358, wherein the combination of bivalve and echinoderm is one of the following: the bivalve is an oyster and the echinoderm is a brittle star; the bivalve is a cockle and the echinoderm is a sea star; the bivalve is a cockle and the echinoderm is a feather star; the bivalve is a mussel and the echinoderm is a sea star; the bivalve is a scallop and the echinoderm is a starfish; the bivalve is a clam and the echinoderm is a starfish; the bivalve is a clam and the echinoderm is a brittle star; the bivalve is an oyster and the echinoderm is a sea star; the bivalve is a cockle and the echinoderm is a feather star; the bivalve is a mussel and the echinoderm is an urchin; the bivalve is a cockle and the echinoderm is a brittle star; the bivalve is a cockle and the echinoderm is an urchin; the bivalve is a mussel and the echinoderm is a brittle star; the bivalve is a scallop and the echinoderm is a brittle star; the bivalve is an oyster and the echinoderm is a starfish; the bivalve is an oyster and the echinoderm is a feather star; the bivalve is an oyster and the echinoderm is an urchin; the bivalve is a cockle and the echinoderm is an urchin; the bivalve is a cockle and the echinoderm is a brittle star; the bivalve is a scallop and the echinoderm is a sea star; the bivalve is a scallop and the echinoderm is an urchin; the bivalve is a clam and the echinoderm is an urchin; the bivalve is a clam and the echinoderm is a feather star; the bivalve is a cockle and the echinoderm is a starfish; the bivalve is a cockle and the echinoderm is a sea star; the bivalve is a mussel and the echinoderm is a feather star; the bivalve is a clam and the echinoderm is a sea star; the bivalve is a cockle and the echinoderm is a starfish; the bivalve is a scallop and the echinoderm is a feather star; or the bivalve is a mussel and the echinoderm is a starfish.
385. The product of EE 384, further comprising an extract from a second bivalve.
386. The product of EE 385, wherein the second bivalve optionally wherein the second bivalve is an oyster.
387. The product of EE 385, wherein the second bivalve is a scallop.
388. The product of EE 385, wherein the second bivalve is a mussel.
389. The product of EE 385, wherein the second bivalve is a cockle.
390. The product of EE 385, wherein the second bivalve is clam.
391. The product of EE 358, wherein the echinoderm is from the class Echinoidea.
392. The product of EE 391, wherein the bivalve is a first bivalve and wherein the product further comprises an extract from a second bivalve.
393. The product of EE 391, wherein the product further comprises collagen or a biodegradable substrate comprising collagen.
394. The product of EE 393, wherein the collagen substrate comprises a plurality of channels.
395. The product of EE 393, wherein the product further comprises a dressing comprising an adhesive, optionally wherein the dressing comprises channels.

396. The product of EE 391, wherein the echinoderm is a heart urchin, a sea urchin, or a sand dollar.
397. The product of EE 391, wherein the bivalve is a clam, an oyster, a cockle, a mussel, a coquina, or a scallop.
398. The product of EE 392, wherein the second bivalve is a clam, an oyster, a cockle, a mussel, a coquina, or a scallop.
399. The product of EE 398, wherein the second bivalve is a clam.
400. A method comprising topically applying a product of EE 358 to a subject in need thereof.
401. The method of EE 400, wherein the subject comprises a wound and the product is applied to the wound.
402. The method of EE 401, wherein the wound is a recalcitrant wound, optionally wherein the recalcitrant wound has failed to be treated successfully with standard of care for treating the wound.
403. The method of EE 401 wherein the wound is chronic skin wound or chronic ulcer.
404. The product of EE 358, wherein the biodegradable substrate comprises collagen in dried or gel form.
405. The product of EE 404, wherein the collagen is in the form of particles or a sheet.
406. The product of EE 404, wherein the substrate comprises the collagen as a majority of the weight of the substrate, a majority of the volume of the substrate, a majority of the dry weight of the substrate, or a majority of the dry volume of the substrate.
407. A product comprising a) a biodegradable substrate; b) a dressing; and optionally c) a therapeutically acceptable bivalve extract and a therapeutically acceptable echinoderm extract.
408. A product comprising a) a biodegradable substrate; b) one or both of a therapeutically acceptable bivalve extract and a therapeutically acceptable echinoderm extract; and optionally c) a dressing.
409. A product comprising a) a biodegradable substrate; b) a dressing; and optionally c) one or both of a therapeutically acceptable bivalve extract and a therapeutically acceptable echinoderm extract.
410. A product comprising a) a biodegradable substrate; b) a dressing; and optionally c) a dermatologically acceptable extract.
411. A product comprising a) a collagen substrate; b) one or both of a therapeutically acceptable bivalve extract and a therapeutically acceptable echinoderm extract; and c) optionally a dressing.
412. The product of any of EEs 358-399, wherein the extract comprises a scallop extract and a sea cucumber extract.
413. The product of EE 412, wherein the scallop extract comprises scallop tissue and/or the sea cucumber extract comprises sea cucumber tissue.
414. The product of EE 413, wherein the sea cucumber tissue in dried form and/or the scallop tissue is in dried form.
415. The product of EE 412, wherein scallop extract is derived from scallop digestive gland and/or scallop gonad.
416. The product of EE 412, wherein sea cucumber extract is derived from sea cucumber digestive gland and/or sea cucumber gonad.
417. The product of EE 414, wherein scallop extract is derived from scallop digestive gland and/or scallop gonad.
418. The product of EE 412, wherein the sea cucumber extract is dehydrated and/or the sea cucumber extract is dehydrated.
419. The product of EE 418, wherein scallop extract is derived from scallop digestive gland and/or scallop gonad.
420. The product of EE 418, wherein sea cucumber extract is derived from sea cucumber digestive gland and/or sea cucumber gonad.
421. The product of EE 420, wherein scallop extract is derived from scallop digestive gland and/or scallop gonad.
422. A product comprising a) a dressing comprising a sheet having channels that allow fluid exchange through the channels; and b) an extract in dried form and comprising at least one wound therapeutic and c) optionally a biodegradable substrate.
423. The product of EE 422, wherein the sheet exhibits a moisture vapor permeability in an area not having channels.
424. A product comprising a) a biodegradable substrate in sheet form and having channels in the sheet that allow fluid exchange through the channels; and b) an extract in dried form and comprising at least one wound therapeutic; and c) optionally a dressing.
425. A product comprising a) a biodegradable substrate in the form of a plurality of particles that allow fluid exchange between the particles; and b) an extract in dehydrated form and comprising at least one wound therapeutic; and c) optionally a dressing.
426. The product of any of EEs 422-425, wherein the extract is lyophilized.
427. The product of any of EEs 422-426, wherein the biodegradable substrate comprises one or more extracellular matrix (‘ECM’) proteins.
428. The product of EE 427, wherein the one or more ECM proteins comprise any one or any combination of a collagen, a fibronectin, a fibrin, a fibrinogen, a laminin, an elastin, a gelatin, and a vitronectin.
429. The product of EE 428, wherein the biodegradable substrate comprises collagen Type I and/or collagen Type III.
430. The product of any of EEs 422-429, wherein the extract comprises bivalve extract, an echinoderm extract, and/or a gastropod extract.
431. The product of any of EEs 422-430, wherein the extract comprises any one or any combination of a scallop extract, a sea cucumber extract, an urchin extract, and an abalone extract.
432. The product of EE 431, wherein the scallop extract comprises scallop tissue and/or the sea cucumber extract comprises sea cucumber tissue.
433. The product of EE 431 or 432, wherein scallop extract is derived from scallop digestive gland and/or scallop gonad.
434. The product of any of EEs 431-433, wherein sea cucumber extract is derived from sea cucumber digestive gland and/or sea cucumber gonad.
435. A method of treating a wound comprising administering the product of any of EEs 422-434 to a subject in need thereof.
436. The product of any of the preceding product EEs, wherein, if the product comprises a substrate, the substrate comprises any one or any combination of technical features taught herein with respect to substrates, except where said one or any combination of technical features taught herein conflicts with the express limitations of the respective EE.
437. The product of any of the preceding product EEs, wherein, if the product comprises a dressing, the dressing comprises any one or any combination of technical features taught herein with respect to dressings, except where said one or any combination of technical features taught herein conflicts with the express limitations of the respective EE
438. The product of any of the preceding product EEs, wherein, if the product comprises an extract, the extract comprises any one or any combination of technical features taught herein with respect to extracts, except where said one or any combination of technical features taught herein conflicts with the express limitations of the respective EE.
439. The method of any of the preceding method EEs, wherein, if the product comprises a substrate, the substrate comprises any one or any combination of technical features taught herein with respect to substrates, except where said one or any combination of technical features taught herein conflicts with the express limitations of the respective EE.
440. The method of any of the method product EEs, wherein, if the product comprises a dressing, the dressing comprises any one or any combination of technical features taught herein with respect to dressings, except where said one or any combination of technical features taught herein conflicts with the express limitations of the respective EE.
441. The method of any of the preceding method EEs, wherein, if the product comprises an extract, the extract comprises any one or any combination of technical features taught herein with respect to extracts, except where said one or any combination of technical features taught herein conflicts with the express limitations of the respective EE.
442. A method of manufacturing the product of any of the preceding product EEs, comprising providing each of the recited components (e.g. extract, substrate, and/or dressing).
443. The method of EE402 wherein, if the product comprises an extract, the extract is produced using any one or any combination of steps taught herein with respect to the production of extracts, except where said one or any combination of steps taught herein conflicts with the express limitations of the respective EE.

1. A product comprising a therapeutically acceptable bivalve extract and an echinoderm extract.

2. The product of claim 1, further comprising a biodegradable substrate.

3. The product of claim 1, further comprising a dressing and optionally, a biodegradable substrate.

4. The product of claim 3, wherein the dressing comprises an adhesive.

5. The product of claim 3, comprising
a. a first layer comprising the biodegradable substrate; and
b. a second layer comprising the dressing
wherein either
i) the first layer further comprises the bivalve extract and the echinoderm extract; or
ii) the product comprises a third layer comprising the bivalve extract and the echinoderm extract.

6. The product of claim 5, wherein the product comprises the three layers and the second layer is between the first layer and the third layer.

7. The product of claim 5, wherein the product comprises the three layers and the third layer is between the second layer and the first layer.

8. The product of claim 3, wherein the dressing is made from a material having a high moisture vapor permeability greater than 2,500 gms/m²/24 hours when measured at 37° C. and 100% to 10% relative humidity and further wherein the dressing contains perforations.

9. The product of claim 2 wherein the biodegradable substrate comprises one or more extracellular matrix (ECM′) proteins.

10. The product of claim 9, wherein the one or more ECM proteins comprise any one or any combination of a collagen, a fibronectin, a fibrin, a fibrinogen, a laminin, an elastin, a gelatin, and a vitronectin.

11. The product of the claim 10, wherein the biodegradable substrate comprises collagen Type I and/or collagen Type III.

12. The product of claim 2, wherein the biodegradable substrate is provided as a plurality of particles or a sheet.

13. (canceled)

14. The product of claim 1, wherein or both of the bivalve extract and the echinoderm extract comprise ground tissue, a polar extract, or a non-polar extract.

15. The product of claim 1, wherein the bivalve is from an order selected from the group consisting of Actinodontida, Adapedonta, Afghanodesmatida, Arcida, Cardiida, Carditida, Colpomyida, Cyrtodontida, Fordillida, Hippuritida, Limida, Lucinida, Megalodontida, Modiomorphida, Myalinida, Myida, Mytilida, Nuculanida, Nuculida, Ostreida, Pectinida, Solemyida, Trigoniida, Tuarangiida, Unionida, and Venerida.

16. The product of claim 15, wherein the bivalve is from the order Pectinida.

17. The product of claim 16, wherein the echinoderm is selected from a starfish, a sea star, a feather star, a brittle star, and an urchin.

18. The product of claim 1, wherein the echinoderm is from a class selected from Asteroidea, Ophiuroidea, Echinoidea, Crinoidea, and Holothuroidea.

19-54. (canceled)

55. The product of claim 1, wherein the bivalve extract comprises a scallop extract and the echinoderm extract comprises a sea cucumber extract, optionally wherein the product further comprises a biodegradable substrate, optionally wherein the biodegradable substrate comprises collagen.

56-66. (canceled)

67. A product comprising a) a biodegradable substrate in sheet form and having channels in the sheet that allow fluid exchange through the channels; and b) an extract in dried form and comprising at least one wound therapeutic; and c) optionally a dressing.

68. A product comprising a) a biodegradable substrate in the form of a plurality of particles that allow fluid exchange between the particles; and b) an extract in dehydrated form and comprising at least one wound therapeutic; and c) optionally a dressing.

69-85. (canceled)
